SEQUENCE LISTING (26 sequences)

SEQ ID NO: 1 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 2 - protein, 805 aa, Homo sapiens [sequence omitted]

SEQ ID NO: 3 - protein, 172 aa, Homo sapiens
METRKPAERLALPYSLRTAPLGVPGTLPGLPRRDPLRVALRLDAACWEWARSGCARGWQYLPVPLDSAFEPAFLRKRNERERQRVRCVNEGYARLRDHLPRELADKRLSKVETLRAAIDYIKHLQELLERQAWGLEGAAGAVPQRRAECNSDGESKASSAPSPSSEPEEGGS

SEQ ID NO: 4 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 5 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 6 - protein, 125 aa, Homo sapiens
MPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 7 - protein, 437 aa, Homo sapiens
MASVAVDPQPSVVTRVVNLPLVSSTYDLMSSAYLSTKDQYPYLKSVCEMAENGVKTITSVAMTSALPIIQKLEPQIAVANTYACKGLDRIEERLPILNQPSTQIVANAKGAVTGAKDAVTTTVTGAKDSVASTITGVMDKTKGAVTGSVEKTKSVVSGSINTVLGSRMMQLVSSGVENALTKSELLVEQYLPLTEEELEKEAKKVEGFDLVQKPSYYVRLGSLSTKLHSRAYQQALSRVKEAKQKSQQTISQLHSTVHLIEFARKNVYSANQKIQDAQDKLYLSWVEWKRSIGYDDTDESHCAEHIESRTLAIARNLTQQLQTTCHTLLSNIQGVPQNIQDQAKHMGVMAGDIYSVFRNAASFKEVSDSLLTSSKGQLQKMKESLDDVMDYLVNNTPLNWLVGPFYPQLTESQNAQDQGAEMDKSSQETQRSEHKTH

SEQ ID NO: 8 - protein, 1857 aa, Homo sapiens
MKMSEGIISPLSLSSESSEQQQAAIRKFSNGSNGSGGGGGSNLSVNSSNSGSNNSIRKSSTLMYNGPLPSINDGKELLLENSKPKVVELVNTFNHKPLSTIHSVHNEIPPPAIEKEKKEIINTISNSGVTKYMTALEILDSTINTPLNRSRSGSIGSKPICNNLTSSSSSSSTTATTPSPTTTSNNNNNNNNNNNNNNNNNNNNNNNNNNNNNNNNNNNNNNNNNNTTSTTTTTTSILISSSPPPSSSSSSSSNDEQFNNNNNNNNSNSGGSSRMITSKSQIKPLIVTSNTAATTTTTTTTNTSAPTTPTNRVQSSLDDLLFNLPTIPSNVPTVNGGPKISAVPKKVSSSKLLIPPSSNVSSSSNITLSLSSSSPSSSSSSSTSTVVPIVQLSSSNSTNSPSTSLPTTPRLSQPTTSYTQLIPSQQQQQPTESNSSSNTNTTTTSSSSSSSSSSLTISSPQPSNNSIRISAFGRSSTQFTISSNGIPSSPGQVSNKDYNNIGNLSNSSGERVKNKQYSMLNISKKTILDESDISSSPRSIGSPNSIRASISSQLPPSLSSIGGGGGGGSGPNVVSNKPLVVKKPSTSEHIKKENIWRQTMIPLTKEDHIKVVFEGIPGKRVIQKFLIDKTPIEIKSKFFEDLKDGDLLNGLTQLPSLIPEHYELKVLSVNSTISNETLPLRRQTLMQACNISRLFPKLHLILKSESTTILDGASTTTTTTTTTTTTTANQSSNIITKSNSSLDLTINNSNEIIDVKGHIQAELEIFELIGTSFTRVLDQGQEVVSFRRDFAQFRLSNFTSTRNDLSQMIYVSSEPLPLTIPNKITIMVLLPGDGKIIKRVDCCPNSSVGDVKKEIFKKFAMIDRVHTQGKTQDDFVLKVTGFREYILCIHELGNLTSRQRFYPTGSGGDFSLMDYDYIRQCVGKNQTVELSLTNNSILSLNQVSEKVSFIDKILETSDFDDYDEDLDSINSNSFDDLKQSIQQQQQQQIQTVINIKETNKENKDSNKENKDSSSNNNNNNNNNNNNNNNNNNNNNNNNNNNGNNNGNNSNNNSNSNISRGSIDSEGNGSGSGNGSEQPTLIGVQNFSLPNNSKLPINIVKRLFRVNIAGLRNLNFNNNEDARNKFADGKNNQPNVFVMAELYYGGELLTNPVFTPIAQLASYGDGSVEFPNWEKGIAFTIPIRYLPRAARASFTVYVTTISEALESQMDEVVSKSIPIGWSNCLLMNHKGMLRMGPTAFRLWDDGRRANPIGTCVDNQAAKQPIILLVEFESFIRPIVYVDTALQSMMVNDSSSISSNGVESPSIVSFSSSAASSSPLPSSPLPSPVGLKKLDLDEARRLKALMDSDPLVQLSAEDKKLVYGYRHIYKSKPKALAKFLLSVNWIDPDQVTDAYRQMNDWALLKPVQALEILDAKFADEHVRNFAIKIINSFSDAEFSDFLLQLTQVLKYEPYHNSDLTHILIQRALSNRSRIGHFFFWFLKSEMHTPEIEERYGLLLEGYLRSCGTHRQDLIKQNQVLKSLHTVAMAVKQTNGSSERKKVLMEGLSKIKFPDTFQLPLDPRWEAKGLIIDKCRYMDSKKLPLWLVFENVEPHAKPLTVIFKVGDDLRQDILTLQVLRIMDKFWKNSGMDLRLQPYKCIATGDGIGMLEVVLNANTIANINKDAGGTGALLEEKTLVNWLKECNKTEAEYNKAVETFILSCAGYVVATYVMGIGDRHSDNIMITKLGHLFHIDFGHFLGNYKKKYGFKRERAPFIFTPQYMAIVGGKDSENFKRFVTTCCSAYNILRKNTDLFINLFQLMLSTGIPELQVAEDIDYLRKALAPGLSDEEAAEEFTKNISVALNTKTVLLNDIFHGWAH

SEQ ID NO: 9 - protein, 703 aa, Homo sapiens
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 10 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 11 - protein, 1279 aa, Homo sapiens
MTLTERLREKISRAFYNHGLLCASYPIPIILFTGFCILACCYPLLKLPLPGTGPVEFTTPVKDYSPPPVDSDRKQGEPTEQPEWYVGAPVAYVQQIFVKSSVFPWHKNLLAVDVFRSPLSRAFQLVEEIRNHVLRDSSGIRSLEELCLQVTDLLPGLRKLRNLLPEHGCLLLSPGNFWQNDWERFHADPDIIGTIHQHEPKTLQTSATLKDLLFGVPGKYSGVSLYTRKRMVSYTITLVFQHYHAKFLGSLRARLMLLHPSPNCSLRAESLVHVHFKEEIGVAELIPLVTTYIILFAYIYFSTRKIDMVKSKWGLALAAVVTVLSSLLMSVGLCTLFGLTPTLNGGEIFPYLVVVIGLENVLVLTKSVVSTPVDLEVKLRIAQGLSSESWSIMKNMATELGIILIGYFTLVPAIQEFCLFAVVGLVSDFFLQMLFFTTVLSIDIRRMELADLNKRLPPEACLPSAKPVGQPTRYERQLAVRPSTPHTITLQPSSFRNLRLPKRLRVVYFLARTRLAQRLIMAGTVVWIGILVYTDPAGLRNYLAAQVTEQSPLGEGALAPMPVPSGMLPPSHPDPAFSIFPPDAPKLPENQTSPGESPERGGPAEVVHDSPVPEVTWGPEDEELWRKLSFRHWPTLFSYYNITLAKRYISLLPVIPVTLRLNPREALEGRHPQDGRSAWPPPGPIPAGHWEAGPKGPGGVQAHGDVTLYKVAALGLATGIVLVLLLLCLYRVLCPRNYGQLGGGPGRRRRGELPCDDYGYAPPETEIVPLVLRGHLMDIECLASDGMLLVSCCLAGHVCVWDAQTGDCLTRIPRPGRQRRDSGVGSGLEAQESWERLSDGGKAGPEEPGDSPPLRHRPRGPPPPSLFGDQPDLTCLIDTNFSAQPRSSQPTQPEPRHRAVCGRSRDSPGYDFSCLVQRVYQEEGLAAVCTPALRPPSPGPVLSQAPEDEGGSPEKGSPSLAWAPSAEGSIWSLELQGNLIVVGRSSGRLEVWDAIEGVLCCSSEEVSSGITALVFLDKRIVAARLNGSLDFFSLETHTALSPLQFRGTPGRGSSPASPVYSSSDTVACHLTHTVPCAHQKPITALKAAAGRLVTGSQDHTLRVFRLEDSCCLFTLQGHSGAITTVYIDQTMVLASGGQDGAICLWDVLTGSRVSHVFAHRGDVTSLTCTTSCVISSGLDDLISIWDRSTGIKFYSIQQDLGCGASLGVISDNLLVTGGQGCVSFWDLNYGDLLQTVYLGKNSEAQPARQILVLDNAAIVCNFGSELSLVYVPSVLEKLD

SEQ ID NO: 12 - protein, 713 aa, Homo sapiens
MLLATLLLLLLGGALAHPDRIIFPNHACEDPPAVLLEVQGTLQRPLVRDSRTSPANCTWLILGSKEQTVTIRFQKLHLACGSERLTLRSPLQPLISLCEAPPSPLQLPGGNVTITYSYAGARAPMGQGFLLSYSQDWLMCLQEEFQCLNHRCVSAVQRCDGVDACGDGSDEAGCSSDPFPGLTPRPVPSLPCNVTLEDFYGVFSSPGYTHLASVSHPQSCHWLLDPHDGRRLAVRFTALDLGFGDAVHVYDGPGPPESSRLLRSLTHFSNGKAVTVETLSGQAVVSYHTVAWSNGRGFNATYHVRGYCLPWDRPCGLGSGLGAGEGLGERCYSEAQRCDGSWDCADGTDEEDCPGCPPGHFPCGAAGTSGATACYLPADRCNYQTFCADGADERRCRHCQPGNFRCRDEKCVYETWVCDGQPDCADGSDEWDCSYVLPRKVITAAVIGSLVCGLLLVIALGCTCKLYAIRTQEYSIFAPLSRMEAEIVQQQAPPSYGQLIAQGAIPPVEDFPTENPNDNSVLGNLRSLLQILRQDMTPGGGPGARRRQRGRLMRRLVRRLRRWGLLPRTNTPARASEARSQVTPSAAPLEALDGGTGPAREGGAVGGQDGEQAPPLPIKAPLPSASTSPAPTTVPEAPGPLPSLPLEPSLLSGVVQALRGRLLPSLGPPGPTRSPPGPHTAVLALEDEDDVLLVPLAEPGVWVAEAEDEPLLT

SEQ ID NO: 13 - protein, 879 aa, Homo sapiens
MKEEAFLRRRFSLCPPSSTPQKVDPRKLTRNLLLSGDNELYPLSPGKDMEPNGPSLPRDEGPPTPSSATKVPPAEYRLCNGSDKECVSPTARVTKKETLKAQKENYRQEKKRATRQLLSALTDPSVVIMADSLKIRGTLKSWTKLWCVLKPGVLLIYKTPKVGQWVGTVLLHCCELIERPSKKDGFCFKLFHPLDQSVWAVKGPKGESVGSITQPLPSSYLIFRAASESDGRCWLDALELALRCSSLLRLGTCKPGRDGEPGTSPDASPSSLCGLPASATVHPDQDLFPLNGSSLENDAFSDKSERENPEESDTETQDHSRKTESGSDQSETPGAPVRRGTTYVEQVQEELGELGEASQVETVSEENKSLMWTLLKQLRPGMDLSRVVLPTFVLEPRSFLNKLSDYYYHADLLSRAAVEEDAYSRMKLVLRWYLSGFYKKPKGIKKPYNPILGETFRCCWFHPQTDSRTFYIAEQVSHHPPVSAFHVSNRKDGFCISGSITAKSRFYGNSLSALLDGKATLTFLNRAEDYTLTMPYAHCKGILYGTMTLELGGKVTIECAKNNFQAQLEFKLKPFFGGSTSINQISGKITSGEEVLASLSGHWDRDVFIKEEGSGSSALFWTPSGEVRRQRLRQHTVPLEEQTELESERLWQHVTRAISKGDQHRATQEKFALEEAQRQRARERQESLMPWKPQLFHLDPITQEWHYRYEDHSPWDPLKDIAQFEQDGILRTLQQEAVARQTTFLGSPGPRHERSGPDQRLRKASDQPSGHSQATESSGSTPESCPELSDEEQDGDFVPGGESPCPRCRKEARRLQALHEAILSIREAQQELHRHLSAMLSSTARAAQAPTPGLLQSPRSWFLLCVFLACQLFINHILK

SEQ ID NO: 14 - protein, 950 aa, Homo sapiens
MNTEAEQQLLHHARNGNAEEVRQLLETMARNEVIADINCKGRSKSNLGWTPLHLACYFGHRQVVQDLLKAGAEVNVLNDMGDTPLHRAAFTGRKELVMLLLEYNADTTIVNGSGQTAKEVTHAEEIRSMLEAVERTQQRKLEELLLAAAREGKTTELTALLNRPNPPDVNCSDQLGNTPLHCAAYRAHKQCALKLLRSGADPNLKNKNDQKPLDLAQGAEMKHILVGNKVIYKALKRYEGPLWKSSRFFGWRLFWVVLEHGVLSWYRKQPDAVHNIYRQGCKHLTQAVCTVKSTDSCLFFIKCFDDTIHGFRVPKNSLQQSREDWLEAIEEHSAYSTHYCSQDQLTDEEEEDTVSAADLKKSLEKAQSCQQRLDREISNFLKMIKECDMAKEMLPSFLQKVEVVSEASRETCVALTDCLNLFTKQEGVRNFKLEQEQEKNKILSEALETLATEHHELEQSLVKGSPPASILSEDEFYDALSDSESERSLSRLEAVTARSFEEEGEHLGSRKHRMSEEKDCGGGDALSNGIKKHRTSLPSPMFSRNDFSIWSILRKCIGMELSKITMPVIFNEPLSFLQRLTEYMEHTYLIHKASSLSDPVERMQCVAAFAVSAVASQWERTGKPFNPLLGETYELVRDDLGFRLISEQVSHHPPISAFHAEGLNNDFIFHGSIYPKLKFWGKSVEAEPKGTITLELLEHNEAYTWTNPTCCVHNIIVGKLWIEQYGNVEIINHKTGDKCVLNFKPCGLFGKELHKVEGYIQDKSKKKLCALYGKWTECLYSVDPATFDAYKKNDKKNTEEKKNSKQMSTSEELDEMPVPDSESVFIIPGSVLLWRIAPRPPNSAQMYNFTSFAMVLNEVDKDMESVIPKTDCRLRPDIRAMENGEIDQASEEKKRLEEKQRAARKNRSKSEEDWKTRWFHQGPNPYNGAQDWIYSGSYWDRNYFNLPDIY

SEQ ID NO: 15 - protein, 569 aa, Homo sapiens
MGSAVMDTKKKKDVSSPGGSGGKKNASQKRRSLRVHIPDLSSFAMPLLDGDLEGSGKHSSRKVDSPFGPGSPSKGFFSRGPQPRPSSPMSAPVRPKTSPGSPKTVFPFSYQESPPRSPRRMSFSGIFRSSSKESSPNSNPATSPGGIRFFSRSRKTSGLSSSPSTPTQVTKQHTFPLESYKHEPERLENRIYASSSPPDTGQRFCPSSFQSPTRPPLASPTHYAPSKAAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 16 - protein, 396 aa, Homo sapiens
MLSEVLLVSAPGKVILHGEHAVVHGKVALAVSLNLRTFLRLQPHSNGKVDLSLPNIGIKRAWDVARLQSLDTSFLEQGDVTTPTSEQVEKLKEVAGLPDDCAVTERLAVLAFLYLYLSICRKQRALPSLDIVVWSELPPGAGLGSSAAYSVCLAAALLTVCEEIPNPLKDGDCVNRWTKEDLELINKWAFQGERMIHGNPSGVDNAVSTWGGALRYHQGKISSLKRSPALQILLTNTKVPRNTRALVAGVRNRLLKFPEIVAPLLTSIDAISLECERVLGEMGEAPAPEQYLVLEELIDMNQHHLNALGVGHASLDQLCQVTRARGLHSKLTGAGGGGCGITLLKPGLEQPEVEATKQALTSCGFDCLETSIGAPGVSIHSATSLDSRVQQALDGL

SEQ ID NO: 17 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 18 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 19 - protein, 1044 aa, Homo sapiens
MPPGVDCPMEFWTKEENQSVVVDFLLPTGVYLNFPVSRNANLSTIKQLLWHRAQYEPLFHMLSGPEAYVFTCINQTAEQQELEDEQRRLCDVQPFLPVLRLVAREGDRVKKLINSQISLLIGKGLHEFDSLCDPEVNDFRAKMCQFCEEAAARRQQLGWEAWLQYSFPLQLEPSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSSILAMRDEQSNPAPQVQKPRAKPPPIPAKKPSSVSLWSLEQPFRIELIQGSKVNADERMKLVVQAGLFHGNEMLCKTVSSSEVSVCSEPVWKQRLEFDINICDLPRMARLCFALYAVIEKAKKARSTKKKSKKADCPIAWANLMLFDYKDQLKTGERCLYMWPSVPDEKGELLNPTGTVRSNPNTDSAAALLICLPEVAPHPVYYPALEKILELGRHSECVHVTEEEQLQLREILERRGSGELYEHEKDLVWKLRHEVQEHFPEALARLLLVTKWNKHEDVAQMLYLLCSWPELPVLSALELLDFSFPDCHVGSFAIKSLRKLTDDELFQYLLQLVQVLKYESYLDCELTKFLLDRALANRKIGHFLFWHLRSEMHVPSVALRFGLILEAYCRGSTHHMKVLMKQGEALSKLKALNDFVKLSSQKTPKPQTKELMHLCMRQEAYLEALSHLQSPLDPSTLLAEVCVEQCTFMDSKMKPLWIMYSNEEAGSGGSVGIIFKNGDDLRQDMLTLQMIQLMDVLWKQEGLDLRMTPYGCLPTGDRTGLIEVVLRSDTIANIQLNKSNMAATAAFNKDALLNWLKSKNPGEALDRAIEEFTLSCAGYCVATYVLGIGDRHSDNIMIRESGQLFHIDFGHFLGNFKTKFGINRERVPFILTYDFVHVIQQGKTNNSEKFERFRGYCERAYTILRRHGLLFLHLFALMRAAGLPELSCSKDIQYLKDSLALGKTEEEALKHFRVKFNEALRESWKTKVNWLAHNVSKDNRQ

SEQ ID NO: 20 - protein, 461 aa, Homo sapiens
MYNTVWSMDRDDADWREVMMPYSTELIFYIEMDPPALPPKPPKPMTSAVPNGMKDSSVSLQDAEWYWGDISREEVNDKLRDMPDGTFLVRDASTKMQGDYTLTLRKGGNNKLIKIYHRDGKYGFSDPLTFNSVVELINHYHHESLAQYNPKLDVKLMYPVSRYQQDQLVKEDNIDAVGKKLQEYHSQYQEKSKEYDRLYEEYTRTSQEIQMKRTAIEAFNETIKIFEEQCHTQEQHSKEYIERFRREGNEKEIERIMMNYDKLKSRLGEIHDSKMRLEQDLKNQALDNREIDKKMNSIKPDLIQLRKIRDQHLVWLNHKGVRQKRLNVWLGIKNEDADENYFINEEDENLPHYDEKTWFVEDINRVQAEDLLYGKPDGAFLIRESSKKGCYACSVVADGEVKHCVIYSTARGYGFAEPYNLYSSLKELVLHYQQTSLVQHNDSLNVRLAYPVHAQMPSLCR

SEQ ID NO: 21 - protein, 602 aa, Homo sapiens
MRSMKALQKALSRAGSHCGRGGWGHPSRSPLLGGGVRHHLSEAAAQGRETPHSHQPQHQDHDSSESGMLSRLGDLLFYTIAEGQERIPIHKFTTALKATGLQTSDPRLRDCMSEMHRVVQESSSGGLLDRDLFRKCVSSNIVLLTQAFRKKFVIPDFEEFTGHVDRIFEDVKELTGGKVAAYIPQLAKSNPDLWGVSLCTVDGQRHSVGHTKIPFCLQSCVKPLTYAISISTLGTDYVHKFVGKEPSGLRYNKLSLNEEGIPHNPMVNAGAIVVSSLIKMDCNKAEKFDFVLQYLNKMAGNEYMGFSNATFQSEKETGDRNYAIGYYLKEKKCFPKGVDMMAALDLYFQLCSVEVTCESGSVMAATLANGGICPITGESVLSAEAVRNTLSLMHSCGMYDFSGQFAFHVGLPAKSAVSGAILLVVPNVMGMMCLSPPLDKLGNSHRGTSFCQKLVSLFNFHNYDNLRHCARKLDPRREGAEIRNKTVVNLLFAAYSGDVSALRRFALSAMDMEQKDYDSRTALHVAAAEGHIEVVKFLIEACKVNPFAKDRWGNIPLDDAVQFNHLEVVKLLQDYQDSYTLSETQAEAAAEALSKENLESMV

SEQ ID NO: 22 - protein, 887 aa, Homo sapiens
MGEAEKFHYIYSCDLDINVQLKIGSLEGKREQKSYKAVLEDPMLKFSGLYQETCSDLYVTCQVFAEGKPLALPVRTSYKAFSTRWNWNEWLKLPVKYPDLPRNAQVALTIWDVYGPGKAVPVGGTTVSLFGKYGMFRQGMHDLKVWPNVEADGSEPTKTPGRTSSTLSEDQMSRLAKLTKAHRQGHMVKVDWLDRLTFREIEMINESEKRSSNFMYLMVEFRCVKCDDKEYGIVYYEKDGDESSPILTSFELVKVPDPQMSMENLVESKHHKLARSLRSGPSDHDLKPNAATRDQLNIIVSYPPTKQLTYEEQDLVWKFRYYLTNQEKALTKFLKCVNWDLPQEAKQALELLGKWKPMDVEDSLELLSSHYTNPTVRRYAVARLRQADDEDLLMYLLQLVQALKYENFDDIKNGLEPTKKDSQSSVSENVSNSGINSAEIDSSQIITSPLPSVSSPPPASKTKEVPDGENLEQDLCTFLISRACKNSTLANYLYWYVIVECEDQDTQQRDPKTHEMYLNVMRRFSQALLKGDKSVRVMRSLLAAQQTFVDRLVHLMKAVQRESGNRKKKNERLQALLGDNEKMNLSDVELIPLPLEPQVKIRGIIPETATLFKSALMPAQLFFKTEDGGKYPVIFKHGDDLRQDQLILQIISLMDKLLRKENLDLKLTPYKVLATSTKHGFMQFIQSVPVAEVLDTEGSIQNFFRKYAPSENGPNGISAEVMDTYVKSCAGYCVITYILGVGDRHLDNLLLTKTGKLFHIDFGYILGRDPKPLPPPMKLNKEMVEGMGGTQSEQYQEFRKQCYTAFLHLRRYSNLILNLFSLMVDANIPDIALEPDKTVKKVQDKFRLDLSDEEAVHYMQSLIDESVHALFAAVVEQIHKFAQYWRK

SEQ ID NO: 23 - protein, 275 aa, Homo sapiens
MTDDKDVLRDVWFGRIPTCFTLYQDEITEREAEPYYLLLPRVSYLTLVTDKVKKHFQKVMRQEDISEIWFEYEGTPLKWHYPIGLLFDLLASSSALPWNITVHFKSFPEKDLLHCPSKDAIEAHFMSCMKEADALKHKSQVINEMQKKDHKQLWMGLQNDRFDQFWAINRKLMEYPAEENGFRYIPFRIYQTTTERPFIQKLFRPVAADGQLHTLGDLLKEVCPSAIDPEDGEKKNQVMIHGIEPMLETPLQWLSEHLSYPDNFLHISIIPQPTD

SEQ ID NO: 24 - protein, 84 aa, Homo sapiens
MALSSQIWAACLLLLLLLASLTSGSVFPQQTGQLAELQPQDRAGARASWMPMFQRRRRRDTHFPICIFCCGCCHRSKCGMCCKT

SEQ ID NO: 25 - protein, 552 aa, Homo sapiens
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQVGAGQRAARADSHSLACWGKGASDRTLWPTAAWSPPPAAVLRLCRSGSGHCWGLRSTLASFACRVATTLPVLEGLGPSLGGGTGS

SEQ ID NO: 26 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

(19) United States
(12) Patent Application Publication
(10) Pub. No.: US 2024/0350475 A1
(43) Pub. Date: Oct. 24, 2024

(54) TRIGONELLINE MITIGATES CARDIOMYOPATHY CAUSED BY SARS-COV-2 SPIKE PROTEIN UNDER OBESITY

(71) Applicant: University of South Carolina, Columbia, SC (US)

(72) Inventors: Wenbin Tan, Columbia, SC (US); Vi Nguyen, Columbia, SC (US); Taixing Cui, Columbia, MO (US)

(73) Assignee: University of South Carolina, Columbia, SC (US)

(21) Appl. No.: 18/440,036

(22) Filed: Feb. 13, 2024

Related U.S. Application Data
(60) Provisional application No. 63/495,823, filed on Apr. 13, 2023.

(51) Int. Cl. A61K 31/455; A61P 9/00

(52) U.S. Cl. CPC A61K 31/455; A61P 9/00

(57) ABSTRACT

Described herein are methods, systems, and pharmaceutical compositions for using Trigonelline (TRG), an alkaloid found in the seeds of many plants, including coffee beans, to attenuate Spike-protein exacerbated lipotoxicity and mitochondria stress in endothelial cells and cardiomyocytes to protect the heart from injuries caused by SARS-CoV-2 Spike protein under obesity.

### STATEMENT REGARDING FEDERALLY SPONSORED RESEARCH

This invention was made with government support under Grant Number P20 GM109091, awarded by the NIH. The government has certain rights in the invention.

### TECHNICAL FIELD

The subject matter disclosed herein is generally directed to methods, systems, and pharmaceutical compositions for using Trigonelline (TRG), an alkaloid found in the seeds of many plants, including coffee beans, to attenuate Spike-protein exacerbated lipotoxicity and mitochondria stress in endothelial cells and cardiomyocytes to protect the heart from injuries caused by SARS-CoV-2 Spike protein under obesity.

### BACKGROUND

As the coronavirus disease 2019 (COVID-19) pandemic continues and the number of recovered COVID-19 patients increases, post-acute sequelae of CoV-2 (PASC) have become a new public health threat and burden in the U.S. and worldwide. Obesity is a major risk factor for PASC. Coronavirus disease 2019 (COVID-19) patients show lipid metabolic alterations, but the mechanism remains unknown.

Today, the spread of Coronavirus disease 2019 (COVID-19) continues. Millions of patients have recovered, but many of them remain sick for a long time. These patients are diagnosed with what is known as long COVID. Long COVID has become a major challenge for doctors. Many long COVID patients develop heart problems.

The inventors believe that spike proteins from the SARS-CoV-2 can damage locations in heart cells related to energy production. For this disclosure, the inventors show that trigonelline is a natural substance that can protect heart from SARS-CoV-2-caused damage in obese patients. Accordingly, it is an object of the present disclosure to use trigonelline to mitigate cardiovascular complications caused by SARS-CoV-2 in acute COVID-19 and PACS patients.

Citation or identification of any document in this application is not an admission that such a document is available as prior art to the present disclosure.

### SUMMARY

The above objectives are accomplished according to the present disclosure by providing in one embodiment, a prophylactic method for preventing heart injuries. The method may include administering an effective dose of trigonelline to a subject to prevent injury caused by at least one virus, wherein administration of trigonelline attenuates: spike-protein exacerbated lipotoxicity and mitochondrial dysfunction. in at least one endothelial cell and at least one cardiomyocyte. Further, the subject may be obese. Still further, the at least one virus may be severe acute respiratory syndrome coronavirus 2. Yet further, the method may attenuate mitochondrial dysfunction caused by low-density lipoprotein-cholesterol. Again still, the effective dose of trigonelline may inhibit at least one spike protein. Even further, the at least one spike protein may cause enhanced lipid deposition on at least one cell membrane. Further yet, the at least one spike protein may be SEQ ID NO: 1. Further again, ferroptiosis may cause the spike-protein exacerbated lipotoxicity. Yet further still, Wortmannin may be administered to inhibit protein-induced necrosis. Further again, the protein induced necrosis may be caused by a protein comprising SEQ ID NO: 8.

In a further embodiment, the current disclosure may provide a method for mitigating virus-induced cardiometabolic pathologies associated with obesity. The method may include administering an effective dose of trigonelline to at least one host cell to prevent injury caused by at least one virus, wherein administration of trigonelline attenuates: spike-protein exacerbated lipotoxicity and mitochondrial dysfunction in at least one endothelial cell and at least one cardiomyocyte. Moreover, the host may be obese. Still again, the at least one virus may be severe acute respiratory syndrome coronavirus 2. Further, the method may attenuate mitochondrial dysfunction caused by low-density lipoprotein-cholesterol. Still yet again, the effective dose of trigonelline may inhibit at least one spike protein. Again further, the at least one spike protein ma cause enhanced lipid deposition on at least one cell membrane. Still again, the at least one spike protein may be SEQ ID NO: 1. Further still, ferroptiosis may cause the spike-protein exacerbated lipotoxicity. Yet further still, Wortmannin may be administered to inhibit protein-induced necrosis. Still again, the protein induced necrosis may be caused by a protein comprising SEQ ID NO: 8.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows Spike protein caused lipid deposition in host cells: at (A) Generation of Spike protein stable cell line; (B) The quantification of Oil Red O staining in the mock, pcDNA, and Spike cells with measurement of an absorbance at 492 nm; (C-E) Histological images showing Oil Red O staining of lipid droplets in the mock (C), pcDNA (D), and Spike (E) cells.

FIG. 2 shows transcriptional profiles of representative biomarkers for lipid metabolism, autophagy, and ferroptosis. Relative mRNA levels of biomarkers involving lipid metabolism (A), autophagy (B), and ferroptosis (C) in the mock, pcDNA, and Spike cells.

FIG. 3 shows Spike protein-exaggerated PA-induced lipotoxicity in host cells: (A) Fluorescent images showing the cell viability in the mock, pcDNA, and Spike cells treated by 750 M PA for 48 h in DMEM containing 5% FBS; and (B) Cell viability curves of mock, pcDNA, and Spike cells in response to PA (750 or 1000 M) treatments for 24 to 72 h.

FIG. 4 shows immunoblot showing the relative changes in levels of representative biomarkers of impaired lipid metabolism, autophagy, and ferroptosis in response to PA overload: (A) Representative immunoblot showing the protein levels of ADRP (SEQ ID NO: 7), LCI/II, Fth1 (SEQ ID NO: 4), Nrf2 (SEQ ID NO: 1), PTGS2 (SEQ ID NO: 5), PI3K-b (SEQ ID NO: 8), SRB1 (SEQ ID NO: 25), and beta-actin in the mock, pcDNA, and Spike cells with or without PA treatment; and (B) The relative quantification of each biomarker in cells.

FIG. 5 shows autophagic/ferroptotic inhibitors attenuated the Spike protein-exaggerated PA-induced necrosis: (A) Fluorescent images showing the cell viability in the mock, pcDNA, and Spike cells; and (B) Quantitative data showing the cell viability in each treatment group.

FIG. 6 shows TRG attenuated the Spike protein exaggerated PA-induced necrosis in H9C2 cells: (A) Quantitative data showing the cell viability of H9C2 cells after being infected by VSV-G or Cov-Spp-S lentivirus followed by PA treatment with or without TRG; (B) Fluorescent images showing the cell viability in the VSV-G- or Cov-Spp-S-infected H9C2 cells after PA treatment with or without TRG in DMEM containing 5% FBS.

FIG. 7 shows Spike-EVs induce mitochondria (Mt) stress in hDMVECs: (A) Size and quantity of fabricated mock-EVs and spike-EVs determined by NTA; (B) Spike protein was detected in the fabricated spike-EVs from HEK cells using western blot. An anti-S2 subunit antibody was used; (C) MitoTracker (Red CMXRos, Cellsignal) and CellRox Green (ThermoFisher) were used to stain Mt (yellow arrowheads) and detect cellular ROS levels (green arrowheads), respectively; (D) Mt density per cell (arbitrary unit, a.u.); (E) Mt diameters (μm); and (F) Quantity of cellular ROS signal per cell (a.u.) among various groups.

FIG. 8 shows Spike-EVs plus LDL-c-induced mitochondria stress and increases in ROS could be reversed by TRG in hDMVECs.

FIG. 9 shows living mitochondria were stained by MitoTracker and nucleus were stained by Hoechst 33342.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +1-5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracorneal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of a pharmaceutical formulation thereof such as any of the methods described in greater detail elsewhere herein.

The inventors have found that Spike protein can (1) impair lipid metabolism in host cells and exacerbate lipotoxicity, and (2) Spike protein exacerbates obesity-induced long-term aberrances in cardiac transcriptional signatures and facilitates development of cardiac fibrosis and perturbation of cardiac function in obesity. Trigonelline (TRG) is an alkaloid that can be found in the seeds of many plants, including coffee beans. The inventors found that TRG can attenuate Spike-protein exacerbated lipotoxicity and mitochondria stress in endothelial cells and cardiomyocytes. Therefore, TRG can be used to protect heart from injuries caused by SARS-CoV-2 Spike protein under obesity. Trigonelline is an alkaloid that can be extracted from Leonurus japonicus Houtt., fenugreek seeds and coffee beans. However, the quantities of trigonelline from those products are not high enough to reach efficacious levels. The function of trigonelline for protection of heart from SARS-CoV-2 damage under obesity is unique for this disclosure. Trigonelline is a natural alkaloid that is enriched in many food-related plants such as coffee beans. Thus its safety profiles have been well tested and approved. It is easy to obtain and formulate for customers. It can attenuate mitochondria dysfunction and cell death caused by SARS-CoV-2 plus LDL-c. It directly targets cardiovascular complications in acute COVID-19 and PACS patients.

Coronavirus disease 2019 (COVID-19) patients show lipid metabolic alterations, but the mechanism remains unknown. The current disclosure aimed to investigate whether the Spike protein of severe acute respiratory syndrome coronavirus 2 (SARS-CoV-2) impairs lipid metabolism in host cells.

The inventors generated a Spike cell line in HEK293 using the pcDNA vector carrying the Spike gene expression cassette. A control cell line was generated using the empty pcDNA vector. Gene expression profiles related to lipid metabolic, autophagic, and ferroptotic pathways were investigated. Palmitic acid (PA)-overload was used to assess lipotoxicity-induced necrosis. As compared with controls, the Spike cells showed a significant increase in lipid depositions in cell membranes as well as dysregulation of expression of a panel of molecules involving lipid metabolism, autophagy, and ferroptosis. The Spike cells showed an upregulation of nuclear factor erythroid 2-related factor 2 (Nrf2 (SEQ ID NO: 1)), a multifunctional transcriptional factor, in response to PA. Furthermore, the Spike cells exhibited increased necrosis in response to PA-induced lipotoxicity compared to control cells in a time- and dose-dependent manner via ferroptosis, which could be attenuated by the Nrf2 (SEQ ID NO: 1) inhibitor trigonelline. The inventors conclude that the Spike protein impairs lipid metabolic and autophagic pathways in host cells, leading to increased susceptibility to lipotoxicity via ferroptosis which can be suppressed by a Nrf2 (SEQ ID NO: 1) inhibitor. This data also suggests a central role of Nrf2 (SEQ ID NO: 1) in Spike-induced lipid metabolic impairments.

Coronavirus disease 19 (COVID-19) is a pandemic viral infection that threatens global public health since the initial outbreak in December 2019 at the epicenter of Wuhan City,

Hubei Province, China. See, Coronavirus Resource Center. COVID-19 Dashboard by the Center for Systems Science and Engineering (CSSE) at Johns Hopkins University (JHU). Available online: coronavirus.jhu.edu/map.html. COVID-19 is caused by the severe acute respiratory syndrome coronavirus 2 (SARS-CoV-2) with high pathogenicity and contagiousness. See, Coronaviridae Study Group of the International Committee on Taxonomy of Viruses. The species Severe acute respiratory syndrome-related coronavirus: Classifying 2019-nCoV and naming it SARS-CoV-2. Nat. Microbiol. 2020, 5, 536-544. SARS-CoV-2 is a positive-sense single-stranded RNA virus that is capable of infecting human beings, together with six other coronaviruses. See Id. SARS-CoV-2 is assumed to be zoonotic and shares 96.3% sequence identity with the bat coronavirus RaTG13. See, Zhou, P.; Yang, X. L.; Wang, X. G.; Hu, B.; Zhang, L.; Zhang, W.; Si, H. R.; Zhu, Y.; Li, B.; Huang, C. L.; et al. A pneumonia outbreak associated with a new coronavirus of probable bat origin. Nature 2020, 579, 270-273. The angiotensin converting enzyme 2 (ACE2 (SEQ ID NO: 2)) is the receptor mediated virus entry into host cells via the SARS-CoV-2 Spike protein. Cleavage of Spike protein by Furin and Transmembrane Serine Protease 2 (TMPRSS2 (SEQ ID NO: 26) facilitates SARS-CoV-2 entry into host cells. See, Walls, A. C.; Park, Y. J.; Tortorici, M. A.; Wall, A.; McGuire, A. T.; Veesler, D. Structure, Function, and Antigenicity of the SARS-CoV-2 Spike Glycoprotein. Cell 2020, 181, 281-292.e6. ACE2 (SEQ ID NO: 2) and TMPRSS2 (SEQ ID NO: 26) are the host determinants during initial infection. See, Ziegler, C. G. K.; Allon, S. J.; Nyquist, S. K.; Mbano, I. M.; Miao, V. N.; Tzouanas, C. N.; Cao, Y.; Yousif, A. S.; Bals, J.; Hauser, B. M.; et al. SARS-CoV-2 receptor ACE2 (SEQ ID NO: 2) is an interferon-stimulated gene in human airway epithelial cells and is enriched in specific cell subsets across tissues. Cell 2020, 181, 1016-1035.e19. Neuropillin (NRP) 1 has been identified as an additional host factor to facilitate SARS-CoV-2 entry upon cleavage by Furin. See, Daly, J. L.; Simonetti, B.; Klein, K.; Chen, K. E.; Williamson, M. K.; Anton-Plagaro, C.; Shoemark, D. K.; Simon-Gracia, L.; Bauer, M.; Hollandi, R.; et al. Neuropilin-1 is a host factor for SARS-CoV-2 infection. Science 2020, 370, 861-865; Cantuti-Castelvetri, L.; Ojha, R.; Pedro, L. D.; Djannatian, M.; Franz, J.; Kuivanen, S.; van der Meer, F.; Kallio, K.; Kaya, T.; Anastasina, M.; et al. Neuropilin-1 facilitates SARS-CoV-2 cell entry and infectivity. Science 2020, 370, 856-860; Moutal, A.; Martin, L. F.; Boinon, L.; Gomez, K.; Ran, D.; Zhou, Y.; Stratton, H. J.; Cai, S.; Luo, S.; Gonzalez, K. B.; et al. SARS-CoV-2 Spike protein co-opts VEGF-A/Neuropilin-1 receptor signaling to induce analgesia. Pain 2020, 162, 243-252. In addition, several intracellular pathological effects of the Spike have been reported. For example, The Spike upregulates expression of the hemeoxygenase-1 (HO-1) in kidney cell lines. See, Singh, R. D.; Barry, M. A.; Croatt, A. J.; Ackerman, A. W.; Grande, J. P.; Diaz, R. M.; Vile, R. G.; Agarwal, A.; Nath, K. A. The spike protein of SARS-CoV-2 induces heme oxygenase-1: Pathophysiologic implications. Biochim. Biophys. Acta (BBA) Mol. Basis Dis. 2022, 1868, 166322. The Spike protein can cause downregulation of ACE2 (SEQ ID NO: 2) and impair endothelial mitochondria functions. See, Lei, Y.; Zhang, J.; Schiavon, C. R.; He, M.; Chen, L.; Shen, H.; Zhang, Y.; Yin, Q.; Cho, Y.; Andrade, L.; et al. SARS-CoV-2 Spike Protein Impairs Endothelial Function via Downregulation of ACE 2. Circ. Res. 2021, 128, 1323-1326. Expression of the Spike protein subunit 1 in lung epithelial cells can result in natural killer cell-reduced degranulation. See, Bortolotti, D.; Gentili, V.; Rizzo, S.; Rotola, A.; Rizzo, R. SARS-CoV-2 Spike 1 Protein Controls Natural Killer Cell Activation via the HLA-E/NKG2A Pathway. Cells 2020, 9, 1975. COVID-19 patients can be asymptomatic or symptomatic. The mortality rate of COVID-19 varies in different geographic locations and patient populations. See, Coronavirus Resource Center. Id. More severe symptoms have been experienced by the patients with metabolic-associated preconditions such as hypertension, cardiovascular disorders (CVD), obesity, and diabetes mellitus (DM). See, Richardson, S.; Hirsch, J. S.; Narasimhan, M.; Crawford, J. M.; McGinn, T.; Davidson, K. W.; Barnaby, D. P.; Becker, L. B.; Chelico, J. D.; Cohen, S. L.; et al. Presenting Characteristics, Comorbidities, and Outcomes Among 5700 Patients Hospitalized with COVID-19 in the New York City Area. JAMA 2020, 323, 2052-2059. One common pathogenic co-factor related to hypertension, obesity, DM, and CVD is hypercholesterolemia. Accumulated evidence shows that COVID-19 directly interplays with dire cardiovascular complications including myocardial injury and heart failure, resulting in elevated risk and adverse outcomes among infected patients. See, Samidurai, A.; Das, A. Cardiovascular Complications Associated with COVID-19 and Potential Therapeutic-Strategies. Int. J. Mol. Sci. 2020, 21, 6790.

COVID-19-associated cardiac complications may become even worse in the setting of cardiometabolic pathologies associated with obesity, although obesity per se is a strong risk factor for severe COVID-19. See, Stefan, N.; Birkenfeld, A. L.; Schulze, M. B. Global pandemics interconnected-Obesity, impaired metabolic health and COVID-19. Nat. Rev. Endocrinol. 2021, 17, 135-149. Our recent studies have shown decreased levels of total cholesterol (TC), low density lipoprotein cholesterol (LDL-c) and high-density lipoprotein cholesterol (HDL-c) in COVID-19 patients, which are associated with disease severity and mortality. See, Fan, J.; Wang, H.; Ye, G.; Cao, X.; Xu, X.; Tan, W.; Zhang, Y. Low-density lipoprotein is a potential predictor of poor prognosis in patients with coronavirus disease 2019. Metabolism 2020, 107, 154243; Wei, X.; Zeng, W.; Su, J.; Wan, H.; Yu, X.; Cao, X.; Tan, W.; Wang, H. Hypolipidemia is associated with the severity of COVID-19; J. Clin. Lipidol. 2020, 14, 297-304; and Cao, X.; Yin, R.; Albrecht, H.; Fan, D.; Tan, W. Cholesterol: A new game player accelerating endothelial injuries caused by SARS-CoV-2? Am. J. Physiol. Metab. 2020, 319, E197-E202. Mechanistically, lipids have been shown to be a critical contributor to transmission, replication, and transportation for some types of viruses. For example, lipid rafts have been reported to be necessary for SARS virus replication. See, Lu, Y.; Liu, D. X.; Tam, J. P. Lipid rafts are involved in SARS-CoV entry into Vero E6 cells. Biochem. Biophys. Res. Commun. 2008, 369, 344-349. Although it has been firmly established that obesity and obesity-related complications are major risk factors for COVID-19 severity, the underlying mechanisms have yet to be determined.

Ferroptosis is an iron-dependent lipid peroxidation-driven cell death. During ferroptosis, acyl-CoA synthetase long-chain family 4 (Ascl4 (SEQ ID NO: 3))-dependent lipid biosynthesis regulates the function of the lipoxygenase for lipid peroxidation. See, Yang, W. S.; Stockwell, B. R. Ferroptosis: Death by Lipid Peroxidation. Trends Cell Biol. 2016, 26, 165-176. Ferritin heavy chain 1 (Fth1 (SEQ ID NO: 4)) functions as iron storage and has ferroxidase activity. See, Chen, X.; Li, J.; Kang, R.; Klionsky, D. J.; Tang, D. Ferroptosis: Machinery and regulation. Autophagy 2021, 17, 2054-2081; Zang, H.; Wu, W.; Qi, L.; Tan, W.; Nagarkatti, P.; Nagarkatti, M.; Wang, X.; Cui, T. Autophagy Inhibition Enables Nrf2 to Exaggerate the Progression of Diabetic Cardiomyopathy in Mice. Diabetes 2020, 69, 2720-2734. Ferroptosis can directly promote cellular inflammation via upregulation of prostaglandin E synthase 2 (PTGS2 (SEQ ID NO: 5)). See, Sun, Y.; Chen, P.; Zhai, B.; Zhang, M.; Xiang, Y.; Fang, J.; Xu, S.; Gao, Y.; Chen, X.; Sui, X.; et al. The emerging role of ferroptosis in inflammation. Biomed. Pharmacother. 2020, 127, 110108. Emerging evidence has shown that multi-organelles involve in ferroptosis including lysosome, mitochondria, endoplasmic reticulum, peroxisomes, and Golgi apparatus where the oxidative stress, lipid synthesis and peroxidation, and oxidated cargo sorting and processing occur. See, Yang, W. S. Id. In particular, autophagy, which is regulated by lipid metabolism, interplays with and promotes ferroptosis. See Chen, X Id. Formation of autophagosomes with engulfed cargo for degradation requires autophagy-related genes (ATGs)-related ubiquitin-like reaction and subsequent microtubule-associated protein 1 light chain 3 beta (LC3 (SEQ ID NO: 6))—involved ubiquitin-like reaction. See, Runwal, G.; Stamatakou, E.; Siddiqi, F. H.; Puri, C.; Zhu, Y.; Rubinsztein, D. C. LC3-positive structures are prominent in autophagydeficient cells. Sci. Rep. 2019, 9, 10147. A lipidated LC3, e.g., LC3 II (SEQ ID NO: 6), resulting from the proteolytic cleavage of LC3 (SEQ ID NO: 6), is associated with autophagosomes, which have been widely used for monitoring autophagic flux process. See Chex, X Id. and Zang, H. Id.

Here, the inventors explored the direct interplays among the Spike protein, lipid metabolism, autophagy, and ferroptosis in host cells. The inventors found that the Spike protein impairs lipid metabolic and autophagic pathways in host cells, leading to increased susceptibility to lipotoxicity most likely via switching on nuclear factor erythroid 2-related factor 2 (Nrf2 (SEQ ID NO: 1))-mediated ferroptosis.

### Methods

### Materials

Human embryonic kidney 293 cells (HEK293) and DMEM medium were purchased from ATCC (ATCC, Manassas, VA, USA). The SARS-CoV-2 S gene (GenBank: QHU36824.1) in fusion with a His tag at c-terminal was synthesized and cloned into a pcDNA3.1 vector (Genscript, Piscataway, NJ, USA). The coding sequence was optimized for expression in human cells. See, Cao, X.; Tian, Y.; Nguyen, V.; Zhang, Y.; Gao, C.; Yin, R.; Carver, W.; Fan, D.; Albrecht, H.; Cui, T.; et al. Spike protein of SARS-CoV-2 activates macrophages and contributes to induction of acute lung inflammation in male mice. FASEB J. 2021, 35, e21801. The vector was referred as pcDNA-Spike with a detailed description. See, Id.

Anti-His tag and LC3 (SEQ ID NO: 6) antibodies were purchased from Millipore Sigma (Millipore Sigma, Burlington, MA, USA). Anti-Adipose Differentiation-Related Protein (ADRP (SEQ ID NO: 7), or Perilipin-2, PLIN2), Nrf2 (SEQ ID NO: 1), PTGS2 (SEQ ID NO: 5), and phosphoinositide-3-Kinase (PI3K (SEQ ID NO: 8))-beta antibodies were obtained from ProteinTech (ProteinTech, Rosemont, IL, USA). Anti-ATG7 (SEQ ID NO: 9) antibody was purchased from Abcam (Abcam, Waltham, MA, USA). Anti-scavenger receptor class B type 1 (SRB1 (SEQ ID NO: 25)) antibody was obtained from Novus (Novus, Centennial, CO, USA). Anti-Fth1 (SEQ ID NO: 4), HRP-anti-rabbit or mouse secondary antibodies, and radioimmunoprecipitation assay (RIPA) lysis buffer were obtained from Santa Cruz Biotech (Santa Cruz Biotech., Inc., Dallas, TX, USA). Primers for real time RT-PCR were synthesized by IDT (IDT, Coralville, IA, USA). RNA extraction kit, RT kit, and SYBR green master mix was obtained from Zymo Research (Zymo, Irvine, CA, USA), Takara Bio USA (Takara Bio, Mountain view, CA, USA), and Bio-Rad (Bio-Rad, Hercules, CA, USA), respectively.

### Generation of the Spike-Protein Stable Expression Cell Line

The HEK293 cells were grown in Dulbecco's Modified Eagle's medium (DMEM) containing 10% fetal bovine serum (FBS). The pcDNA-Spike or pcDNA vector was transfected into the cells using lipofectamine 300 reagent (ThermoFisher, Waltham, MA, USA). Two days after transfection, the cells were treated by G418 starting from the concentration of 100 g/mL with a gradual increase to 800 g/mL during the following 2 weeks. The individual colonies with stable integration of the pcDNA-Spike (HEK_Spike) or pcDNA vector (HEK_pcDNA) were selected and expanded. The HEK_Spike stable colonies were confirmed to express the Spike protein using immunoblot. The cells were maintained in DMEM with 10% FBS regularly for further experiments.

### Lipid (Oil Red O) Staining

The Lipid (Oil Red O) kit was obtained from Millipore Sigma. The HEK293, HEK_pcDNA, and HEK_Spike cells were fixed by 10% formalin and followed by 60% isopropanol treatment, followed by an incubation of the Oil Red O working solution for 15 min. The cell nucleus was counterstained using hematoxylin. The images were acquired using an ImagXpress Pico Automated system (Molecular Device, San Jose, CA, USA). Oil Red O stain was extracted in isopropanol followed by a measurement of absorbance at 492 nm using a Thermo Scientific Multiskan Spectrophotometer system.

### Real Time-RT PCR and Western Blot

RT was performed in a 20-L reaction containing 1.0-5 g of total RNA, 0.5 mM dNTPs, 0.5 g of oligo (dT) 15-mer primer, 20 units of RNasin, and 5 units of SMART Moloney murine leukemia virus reverse transcriptase) in 1× RT buffer (Clontech, Mountain View, CA, USA) at 42 C for 2 h. A converted index for three reference genes were used to normalize the amplification data: GAPDH, Nono, and -actin. Expression levels of a panel of 83 genes related to lipid metabolic, autophagic, and ferroptotic pathways were determined using real time RT-PCR. Briefly, the real time PCRs reactions were carried out in a 25-L total volume containing 10 ng of each cDNA template and 10 pmol of each specific primer in 1 SYBR Green qPCR Master Mix (Bio-Rad) with a duplication of each reaction. The PCR parameters include one cycle of 95 C for 2 min and 45 cycles of 95° C. for 15 s at and 60 C for 60 s. The cycle number at which fluorescence crossed the cycle threshold (Ct) for the target and reference genes was used to evaluate the amplification efficiency for the relative quantification of the real time PCR.

For Western Blot assay, cellular proteins were extracted from HEK, HEK_pcDNA, or HEK_Spike cells using RIPA lysis buffer (Santa Cruz Biotech., Inc.), separated by SDS-PAGE, followed by a transfer onto PVDF membranes. Primary antibodies were added and followed by HRP-labeled secondary antibodies. Images were acquired using a Bio-Rad Gel Imaging System.

### Palmitic Acid (PA)-Induced Lipotoxicity Assay

The HEK293, HEK_pcDNA, and HEK_Spike cells were cultured in a 96-well plate and reached to 80% confluence on the next day before treatment. The cells were kept in DMEM medium with 5% FBS during the entire treatment procedure. The cells were treated with PA (free BSA) in various concentrations from 250 to 1000 M for 24, 48, or 72 h.

For treatment control groups, the cells were treated by the same concentrations of BSA in parallel. For the inhibitory assay, the cells were pre-incubated by autophagic inhibitors (125 M trigonelline (TRG), or 10 M Wortmannin), or a ferropotosis inhibitor (ferrostatin, 2 M), or an apoptosis inhibitor (necrostatin-1, 10 M) in DMEM medium with 5% FBS for 2 h, followed by 750 M PA treatment for 24 h. The cells were then co-stained by Propidium Iodide (PI) and Hoechst 33342 NucBlue Live Cell Stain dye (ThermoFisher) to show the dead and live cells. The dyes were excited at 535 nm and 405 nm, respectively. TRG may be provided over a range of concentration such as 50 to 300 M, 60 to 290, 70 to 280, 80 to 270, 90 to 260, 100 to 250, 110 to 240, 120 to 230, 130 to 220, 140 to 210, 150 to 200, 160 to 190, 170 to 180, as well as sub ranges such as from 100M to 200 M, 125M to 175 M, etc., also include individual concentrations, such as 125M, 150 M, 175M, etc. Wortmannin may be provided over a range of concentrations from 5 to 100, 10 to 90, 15 to 80, 20 to 70, 25 to 60, 30 to 50 M, etc., including subranges and individual concentrations within these ranges.

Images were acquired and cell viability analyses were performed using an ImagXpress Pico Automated system.

### Viral Production and H9C2 Cell Culture

The Spike gene was cleaved from pcDNA-Spike plasmid and cloned into lentiviral vector pLV-mCherry (Addgene, Watertown, MA, USA) with removal of mCherry gene to generate pLV-Spike plasmid. The lentiviral production followed our previous report with slight modifications. See, Cao, X.; Tian, Y.; Nguyen, V.; Zhang, Y.; Gao, C.; Yin, R.; Carver, W.; Fan, D.; Albrecht, H.; Cui, T.; et al. Spike protein of SARS-CoV-2 activates macrophages and contributes to induction of acute lung inflammation in male mice. FASEB J. 2021, 35, e21801. The inventors generated a Spike-pseudotyped (Spp) lentivirus with the Spike protein as the viral surface tropism as well as expression of the Spike protein, which was referred to as Cov-Spp-S virus. Briefly, Phoenix cells (ATCC) were cultured in DMEM containing 10% FBS and transfected by pLV-Spike using a calcium phosphate kit (ThermoFisher). The control virus with VSV-G as the tropism and expression of mCherry was generated by co-transfection of pLV-mCherry and pMD2.G vector (Addgene) into the Phoenix cells, which was referred to as VSV-G virus. Seventy-two hours post transfection—the supernatant containing released virus was collected, clarified by centrifuging at 5000 g for 15 min, passed through a 0.45 m filter disk, followed by ultracentrifugation at 24,000 rpm for 2 h using Beckman SW41 rotor. The precipitated virus was resuspended in cold PBS buffer, aliquoted, and stored at 0.80° C. before use. The viral titer was to quantify the RNA copies of the Spike or mCherry using a real time RT-PCR assay.

H9C2 cells (ATCC) were cultured in DMEM (10% FBS) medium in a 96 well-plate with 80% confluence. The cells were infected twice at the first and second days using—Cov-Spp-S or VSV-G lentivirus (4.8×10⁷ particles per well) with addition of polybrene (1 g/mL); the cells were then incubated for an additional 5 days, followed by treatment with PA (675 M) for 24 h in DMEM medium containing 5% FBS. The same concentration of BSA was used for treatment control groups in parallel. For the inhibitory assay, the cells were pre-incubated with 125 M TRG in DMEM medium with 5% FBS for 2 h, followed by 675 M PA treatment for 24 h. The cells were then co-stained with PI and Hoechst 33342 NucBlue Live Cell Stain dye to show the dead and live cells, respectively.

### Statistical Analyses

Origin 2019 was used for statistical analysis. The Student's t test or one-way ANOVA was used for two groups or multiple comparisons test. The data was presented as “mean s.d.” and p<0.05 was considered as significant.

### Results

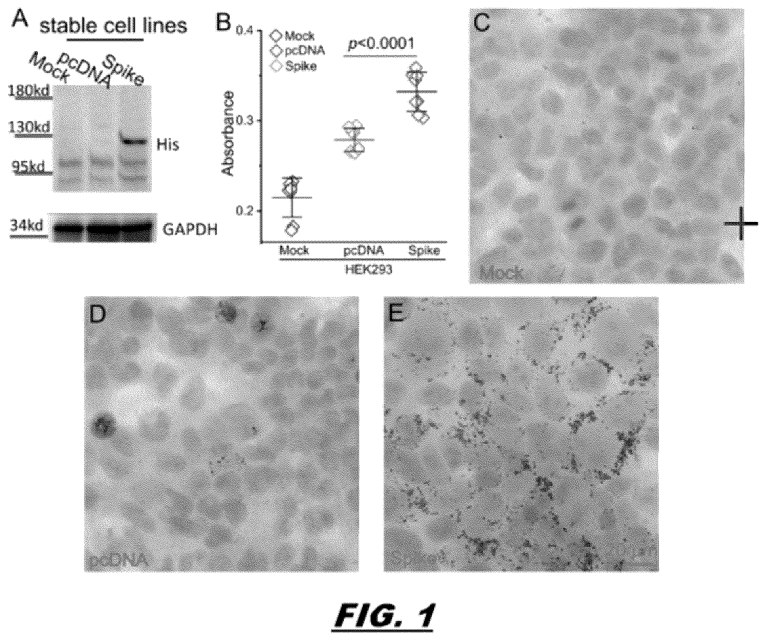

The stable cell line with expression of the Spike protein was obtained upon neomycin selection after transfection of pcDNA_Spike into HEK293 cells. The control stable cell line with integration of the empty pcDNA was generated simultaneously. The expression of the Spike protein was verified using an anti-His tag antibody. See FIG. 1 at A. The same pcDNA_Spike vector was used for production of the Spike protein-pseudotyped (Spp) lentivirus in HEK Phoenix cells in our previous study where the expression of the Spike protein was verified using both anti-Spike S1 and S2 subunit antibodies. See, Cao, X. Id. The inventors did not observe a significant difference in growth rate between pcDNA and Spike stable cell lines. To explore whether the Spike protein had a role in lipid metabolisms in host cells, The inventors performed Oil Red O staining among these cell lines. The inventors found there was a significant accumulation of lipid deposition in the Spike cells compared to pcDNA and mock control cells. See FIG. 1 at B. The histological staining showed that the lipid depositions were mainly located on the cell membrane in the Spike cell line, See FIG. 1 at C-E, indicating an impairment of lipid metabolism in host cells. FIG. 1 shows Spike protein caused lipid deposition in host cells: (A) Generation of Spike protein stable cell line. The expression of Spike protein was verified using an anti-His tag antibody in the Spike cells. (B) The quantification of Oil Red O staining in the mock, pcDNA, and Spike cells with measurement of an absorbance at 492 nm. (C-E) Histological images showing Oil Red O staining of lipid droplets in the mock (C), pcDNA (D), and Spike (E) cells. Nuclear components were counterstained by hematoxylin.

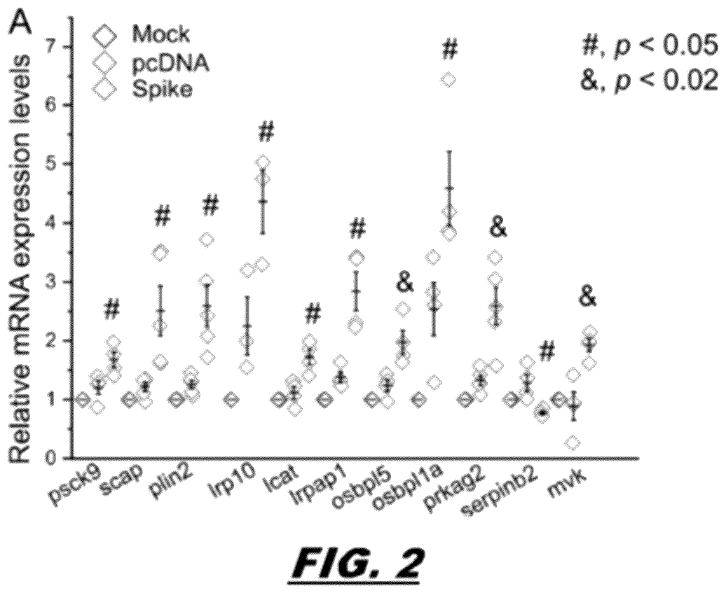

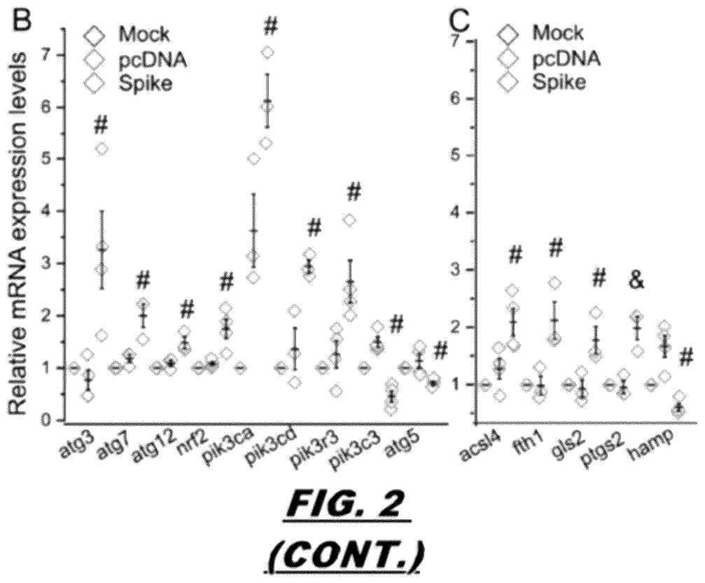

The inventors next examined transcriptional levels of a panel of 83 genes that are representative markers of lipid metabolism, autophagy, and ferroptosis. The inventors found the mRNA levels of many lipid metabolic markers were upregulated such as proprotein convertase subtilisin/kexin type 9 (Psck9 (SEQ ID NO: 10)), SREBF chaperone (Scap (SEQ ID NO: 11), Plin2, low density lipoprotein receptor-related protein 10 (Irp10) (SEQ ID NO: 12), lecithin-cholesterol acyltransferase (lcat), low density lipoprotein receptor-related protein associated protein 1 (Irpap1), oxysterol binding protein-like 5 (Osbp15 (SEQ ID NO: 13)), oxysterol binding protein-like 1A (Osbplla (SEQ ID NO: 14)), protein kinase, AMPactivated, gamma 2 non-catalytic subunit (Prkag2 (SEQ ID NO: 15)), and mevalonate kinase (Mvk (SEQ ID NO: 16); while Serpinb2 (SEQ ID NO: 17) was downregulated in the Spike cells. See FIG. 2 at A. For the examined autophagic and ferroptotic markers, Atg3, Atg7 (SEQ ID NO: 9), Atg12, Nrf2 (SEQ ID NO: 1), Phosphatidylinositol-4,5-Bisphosphate 3-Kinase Catalytic Subunit Alpha (Pik3ca) (SEQ ID NO: 18), Pik3cd (SEQ ID NO: 19), Phosphoinositide-3-Kinase Regulatory Subunit 3 (Pik3r3 (SEQ ID NO: 20), Acs14, Fth1 (SEQ ID NO: 4), glutaminase 2 (Gls2 (SEQ ID NO: 21)), and Ptgs2 (SEQ ID NO: 5) showed an increase in mRNA levels in the Spike cells; while Pik3c3 (SEQ ID NO: 22), Atg5 (SEQ ID NO: 23), and Hamp (SEQ ID NO: 24) showed a decrease in mRNA levels in the Spike cells. See FIG. 2 at B, C. These results reveal a negative impact of Spike proteins per se on lipid metabolism, autophagy, and probably ferroptosis in the cell. FIG. 2 shows transcriptional profiles of representative biomarkers for lipid metabolism, autophagy, and ferroptosis. Relative mRNA levels of biomarkers involving lipid metabolism (A), autophagy (B), and ferroptosis (C) in the mock, pcDNA, and Spike cells. The normalization was performed using a converted index of -actin, nono, and GAPDH as the reference genes.

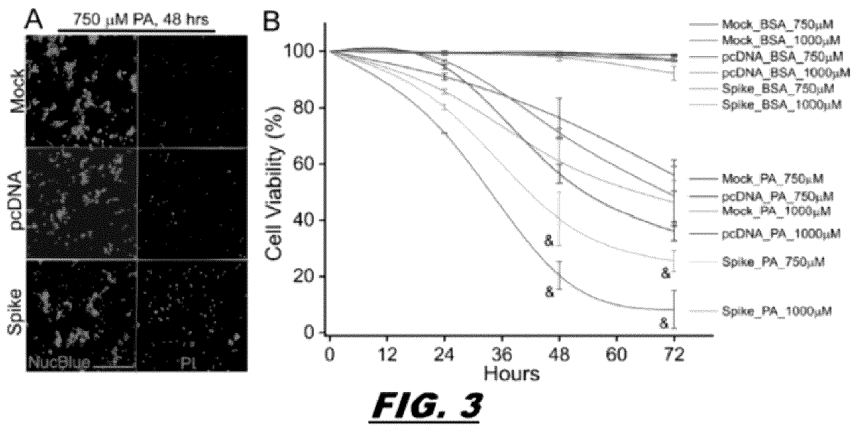

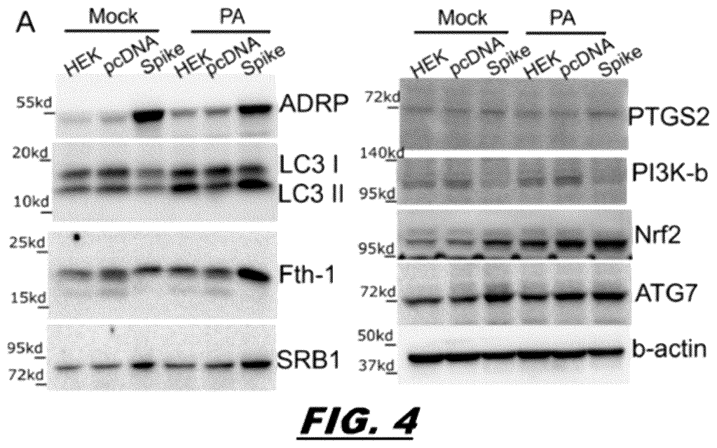

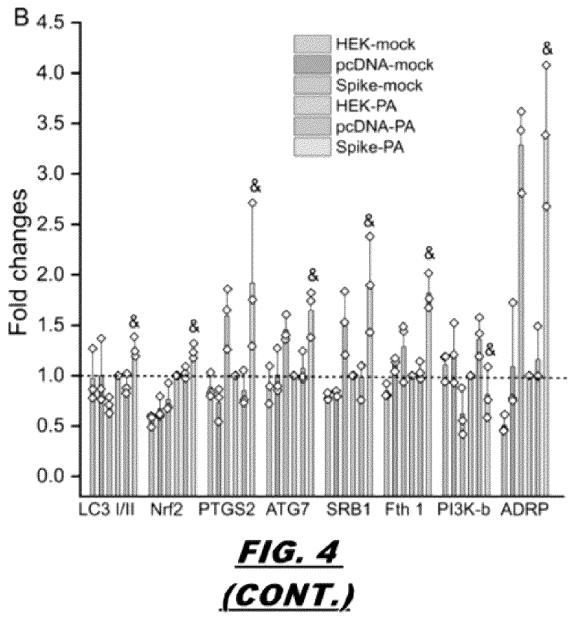

The inventors further examined the biological significance of Spike protein expression with a focus on lipotoxicity in vitro. PA overload induced cell death in cultured HNK293 and pcDNA cells, see FIG. 3 and S1), demonstrating a PA-induced lipotoxicity as described elsewhere. See, Cuthbertson D J, Steele T, Wilding J P, Halford J C, Harrold J A, Hamer M and Karpe F. What have human experimental overfeeding studies taught us about adipose tissue expansion and susceptibility to obesity and metabolic complications? Int J Obes (Lond). 2017; 41:853-865. However, the PA-induced cell death was significantly augmented in Spike cells, see FIG. 3 and S1. The PA-induced cell death also showed a dose- and time-dependent response, see FIG. 3 and S1. The BSA control did not cause any significant cell death among these cell lines. The inventors next examined the expression patterns of some crucial factors in response to PA-induced lipotoxicity. SRB1 (SEQ ID NO: 25), ATG7 (SEQ ID NO: 9), PTGS2 (SEQ ID NO: 5), showed a significantly higher level in the Spike cells than the mock and pcDNA control cells, see FIG. 4, which were consistent with the changes in mRNA levels. See FIG. 2. In response to PA treatment, the levels of SRB1 (SEQ ID NO: 25), ATG7 (SEQ ID NO: 9), PTGS2 (SEQ ID NO: 5), LC3 I/II) (SEQ ID NO: 6) ratio, and Fth1 (SEQ ID NO: 4) in Spike cells increased significantly as compared with the pcDNA control cells. See, FIG. 4. FIG. 3 shows Spike protein-exaggerated PA-induced lipotoxicity in host cells: (A) Fluorescent images showing the cell viability in the mock, pcDNA, and Spike cells treated by 750 M PA for 48 h in DMEM containing 5% FBS. The cells were co-stained by Propidium Iodide (PI) and Hoechst 33342 NucBlue Live Cell Stain dye to show the dead and live cells, respectively. Scale bar, 25 m. (B) Cell viability curves of mock, pcDNA, and Spike cells in response to PA (750 or 1000 M) treatments for 24 to 72 h. & indicates p<0.05 in the Spike cells as compared with the mock and pcDNA control cells in response to the same dosages of PA at 48 or 72 h. n=5˜6 wells in each dose and time point. FIG. 4 shows Immunoblot showing the relative changes in levels of representative biomarkers of impaired lipid metabolism, autophagy, and ferroptosis in response to PA overload. The mock, pcDNA, and Spike cells were treated by 750 M PA for 24 h in DMEM containing 5% FBS. (A) Representative immunoblot showing the protein levels of ADRP (SEQ ID NO: 7), LCI/II, Fth1 (SEQ ID NO: 4), Nrf2 (SEQ ID NO: 1), PTGS2 (SEQ ID NO: 5), PI3K-b (SEQ ID NO: 8), SRB1 (SEQ ID NO: 25), and beta-actin in the mock, pcDNA, and Spike cells with or without PA treatment. (B) The relative quantification of each biomarker in cells. The protein level for each biomarker was first normalized to the corresponding beta-actin level. The relative ratio of each biomarker within group was then obtained using its' level in the mock group with PA treatment as 1 (indicated by the dashed line). & indicates p<0.05 in the Spike cells as compared with the pcDNA control cells with the PA treatment. n=3 independent experiments.

The inventors have previously shown that Nrf2 (SEQ ID NO: 1) is the crucial transcriptional factor mediating PA induced ferroptosis in autophagy-impaired in cardiomyocytes under obese conditions. See, Mizunoe Y, Kobayashi M, Tagawa R, Nakagawa Y, Shimano H and Higami Y. Association between Lysosomal Dysfunction and Obesity-Related Pathology: A Key Knowledge to Prevent Metabolic Syndrome. Int J Mol Sci. 2019; 20. PI3K (SEQ ID NO: 8) has been shown to play a critical role in autophagy. See, Li Q, Mitchell P, Dowling R, Buttery R and Yan B. Neurological picture: A case of cerebral and retinal vascular anomaly in a patient with Klippel-Trenaunay-Weber syndrome. Journal of neurology, neurosurgery, and psychiatry. 2011; 82:1049-50; Puelles V G, Lutgehetmann M, Lindenmeyer M T, Sperhake J P, Wong M N, Allweiss L, Chilla S, Heinemann A, Wanner N, Liu S, Braun F, Lu S, Pfefferle S, Schroder A S, Edler C, Gross O, Glatzel M, Wichmann D, Wiech T, Kluge S, Pueschel K, Aepfelbacher M and Huber T B. Multiorgan and Renal Tropism of SARS-CoV-2. N Engl J Med. 2020; 383:590-592; and Puntmann V O, Carerj M L, Wieters I, Fahim M, Arendt C, Hoffmann J, Shchendrygina A, Escher F, Vasa-Nicotera M, Zeiher A M, Vehreschild M and Nagel E. Outcomes of Cardiovascular Magnetic Resonance Imaging in Patients Recently Recovered From Coronavirus Disease 2019 (COVID-19). JAMA cardiology. 2020.

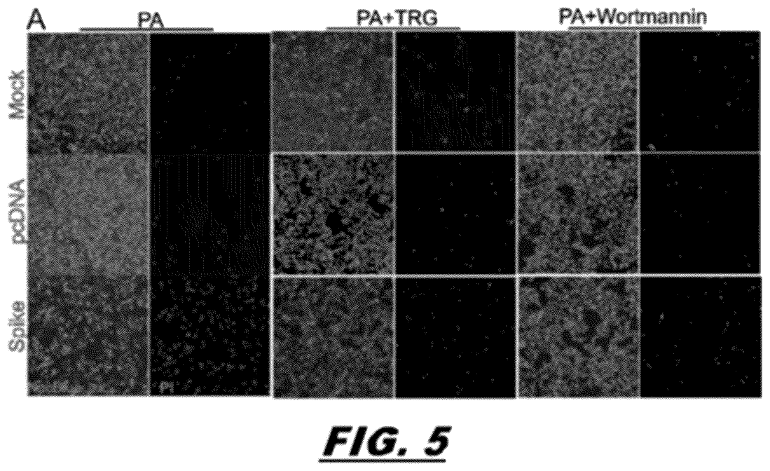

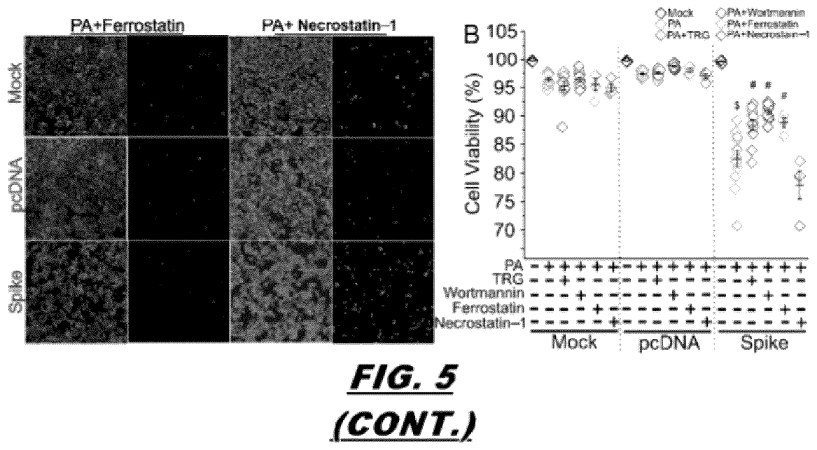

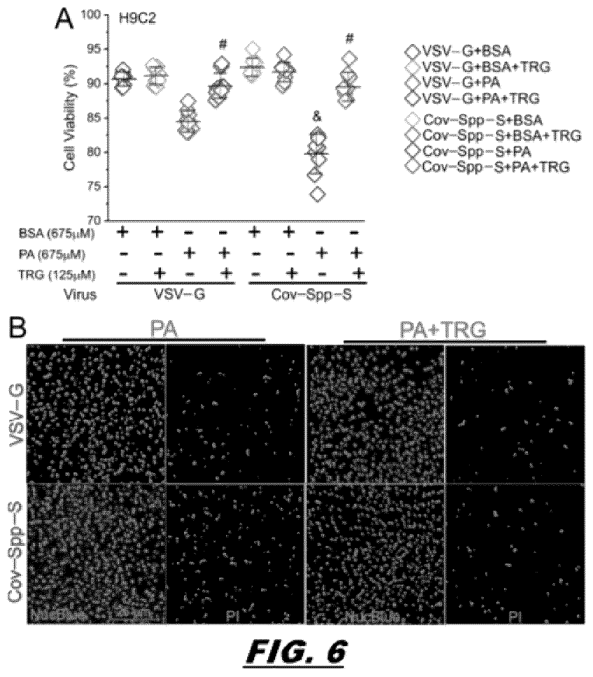

The inventors then asked whether inhibitors for Nrf2 (SEQ ID NO: 1), PI3K (SEQ ID NO: 8), and ferroptosis could attenuate PA-induced lipotoxicity in the Spike cells. Indeed, the Nrf2 (SEQ ID NO: 1) inhibitor TRG, PI3K (SEQ ID NO: 8) pan inhibitor Wortmannin, and ferroptosis inhibitor ferrostatin, but not a necroptosis inhibitor necrostatin 1, significantly mitigated the PA-induced Spike protein-exaggerated lipotoxicity. See FIG. 5. Finally, the inventors attempted to test whether a similar mechanism could be recapitulated in H9C2 cells, a cardiomyocyte-like cell line. The inventors infected the H9C2 cells using CoV-Spp-S lentivirus expressing the Spike protein or VSV-G control virus expressing mCherry. The cells infected by the CoV-Spp-S showed a significantly higher cell death than the cells infected by the VSV-G control virus. See FIG. 6. The PA-induced Spike protein-exaggerated lipotoxicity could be reversed using the Nrf2 (SEQ ID NO: 1) inhibitor TRG. See, FIG. 6. FIG. 5 shows autophagic/ferroptotic inhibitors attenuated the Spike protein-exaggerated PA-induced necrosis. (A) Fluorescent images showing the cell viability in the mock, pcDNA, and Spike cells treated by 750 M PA treatment for 24 h in DMEM containing 5% FBS with or without a Nrf2 (SEQ ID NO: 1) inhibitor TRG, pan-PI3K (SEQ ID NO: 8) inhibitor Wortmannin, ferroptosis inhibitor Ferrostain, and apoptotic inhibitor Necrostatin 1. The cells were co-stained by Propidium Iodide (PI) and Hoechst 33342 NucBlue Live Cell Stain dye to show the dead and live cells, respectively. (B) Quantitative data showing the cell viability in each treatment group. $ indicates p<0.05 in the Spike cells treated by PA as compared with the Spike cells without PA treatment. # indicates p<0.05 in each group treated with PA and various inhibitors as compared with the Spike cells treated with PA only. FIG. 6 shows TRG attenuated the Spike protein exaggerated PA-induced necrosis in H9C2 cells: (A) Quantitative data showing the cell viability of H9C2 cells after being infected by VSV-G or Cov-Spp-S lentivirus followed by PA treatment with or without TRG. BSA was used as a treatment control. &, indicates p<0.05 in the Cov-Spp-S-infected cells as compared with the VSV-G-infected cells upon PA treatment. #, indicates p<0.05 in the cells treated with PA plus TRG as compared with the cells treated with PA only. (B) Fluorescent images showing the cell viability in the VSV-G- or Cov-Spp-S-infected H9C2 cells after PA treatment with or without TRG in DMEM containing 5% FBS. The cells were co-stained by Propidium Iodide (PI) and Hoechst 33342 NucBlue Live Cell Stain dye to show the dead and live cells.

### DISCUSSION

The data in this disclosure have demonstrated that the Spike protein alone can directly impair lipid metabolic and autophagic pathways in host cells, leading to increased lipotoxicity through ferroptosis. This result has shown a direct and evident role of the Spike protein in exaggeration of pre-existing lipotoxicity, revealing a mechanistic insight into the clinical manifestations of high susceptibility and mortality rate of obese patients with COVID-19.

Furthermore, the inventors have shown that the Spike protein-induced necrosis can be suppressed by PI3K (SEQ ID NO: 8) pan inhibitor Wortmannin, ferroptosis inhibitor ferrostatin 1, and Nrf2 (SEQ ID NO: 1) inhibitor TRG. TRG, an alkaloid enriched in coffee, is among those effective inhibitors, providing a potential and feasible preventive strategy to mitigate COVID-19-associated cardiometabolic pathologies associated with obesity.

Numerous studies have reported lipidomic dysregulation in COVID-19 patients. For example, Shen et al. showed a strong downregulation of over 100 lipids including sphingolipids, glycerophospholipids, fatty acids, and various apolipoproteins in COVID-19 patients. See, O'Donnell M A, Perez-Jimenez E, Oberst A, Ng A, Massoumi R, Xavier R, Green D R and Ting A T. Caspase 8 inhibits programmed necrosis by processing CYLD. Nat Cell Biol. 2011; 13:1437-42. Increased levels of sphingomyelins (SMs), non-esterified fatty acids (NEFAs), and free poly-unsaturated fatty acids (PUFAs) have been shown in COVID-19 patients as well. See, Noyan-Ashraf M H, Shikatani E A, Schuiki I, Mukovozov I, Wu J, Li R K, Volchuk A, Robinson L A, Billia F, Drucker D J and Husain M. A glucagon-like peptide-1 analog reverses the molecular pathology and cardiac dysfunction of a mouse model of obesity. Circulation. 2013; 127:74-85; Zang H, Wu W, Qi L, Tan W, Nagarkatti P, Nagarkatti M, Wang X and Cui T. Autophagy Inhibition Enables Nrf2 to Exaggerate the Progression of Diabetic Cardiomyopathy in Mice. Diabetes. 2020; 69:2720-2734.; and Yamanaka S, Sato Y, Oikawa D, Goto E, Fukai S, Tokunaga F, Takahashi H and Sawasaki T. Subquinocin, a small molecule inhibitor of CYLD and USP-family deubiquitinating enzymes, promotes NF-kappaB signaling. Biochem Biophys Res Commun. 2020; 524:1-7. Increases in PLA2 activation, which results in long-chain PUFAs, may be associated with the COVID-19 deterioration. See, Jeffery E, Wing A, Holtrup B, Sebo Z, Kaplan J L, Saavedra-Pena R, Church C D, Colman L, Berry R and Rodeheffer M S. The Adipose Tissue Microenvironment Regulates Depot-Specific Adipogenesis in Obesity. Cell Metab. 2016; 24:142-50; and Frazier T, Lee S, Bowles A, Semon J, Bunnell B, Wu X and Gimble J. Gender and age-related cell compositional differences in C57BL/6 murine adipose tissue stromal vascular fraction. Adipocyte. 2018; 7:183-189. The inventors' previous studies together with other reports have shown the downregulation of serum LDL-c and HDL-c levels in COVID-19 patients. See, 15. Daly J L, Simonetti B, Klein K, Chen K E, Williamson M K, Anton-Plagaro C, Shoemark D K, Simon-Gracia L, Bauer M, Hollandi R, Greber U F, Horvath P, Sessions R B, Helenius A, Hiscox J A, Teesalu T, Matthews D A, Davidson A D, Collins B M, Cullen P J and Yamauchi Y. Neuropilin-1 is a host factor for SARS-CoV-2 infection. Science. 2020; Cantuti-Castelvetri L, Ojha R, Pedro L D, Djannatian M, Franz J, Kuivanen S, van der Meer F, Kallio K, Kaya T, Anastasina M, Smura T, Levanov L, Szirovicza L, Tobi A, Kallio-Kokko H, Osterlund P, Joensuu M, Meunier F A, Butcher S J, Winkler M S, Mollenhauer B, Helenius A, Gokce O, Teesalu T, Hepojoki J, Vapalahti O, Stadelmann C, Balistreri G and Simons M. Neuropilin-1 facilitates SARS-CoV-2 cell entry and infectivity. Science. 2020; Moutal A, Martin L F, Boinon L, Gomez K, Ran D, Zhou Y, Stratton H J, Cai S, Luo S, Gonzalez K B, Perez-Miller S, Patwardhan A, Ibrahim M M and Khanna R. SARS-CoV-2 Spike protein co-opts VEGF-A/Neuropilin-1 receptor signaling to induce analgesia. Pain. 2020; and Ruggiero C, Ehrenshaft M, Cleland E and Stadler K. High-fat diet induces an initial adaptation of mitochondrial bioenergetics in the kidney despite evident oxidative stress and mitochondrial ROS production. Am J Physiol Endocrinol Metab. 2011; 300: E1047-58. These lines of accumulated evidence have revealed a central role of lipids and lipid metabolism in the development of COVID-19.

Here, the inventors demonstrate that the Spike protein executes a direct function in altering lipidome via upregulation of a panel of genes involving lipid metabolism and resulting in enhanced lipid deposition on the cell membrane. This data provides direct evidence showing that the Spike protein modulates lipid metabolism in host cells and is an important independent factor contributing to the altered lipidome in COVID-19 patients.

PI3Ks (SEQ ID NO: 8) play important roles in autophagy formation during early stages of viral infection for both canonical and non-canonical endocytic pathways. See, Li Q, Mitchell P, Dowling R, Buttery R and Yan B. Neurological picture: A case of cerebral and retinal vascular anomaly in a patient with Klippel-Trenaunay-Weber syndrome. Journal of neurology, neurosurgery, and psychiatry. 2011; 82:1049-50; Puelles V G, Lutgehetmann M, Lindenmeyer M T, Sperhake J P, Wong M N, Allweiss L, Chilla S, Heinemann A, Wanner N, Liu S, Braun F, Lu S, Pfefferle S, Schroder A S, Edler C, Gross O, Glatzel M, Wichmann D, Wiech T, Kluge S, Pueschel K, Aepfelbacher M and Huber T B. Multiorgan and Renal Tropism of SARS-CoV-2. N Engl J Med. 2020; 383:590-592; and Puntmann V O, Carerj M L, Wieters I, Fahim M, Arendt C, Hoffmann J, Shchendrygina A, Escher F, Vasa-Nicotera M, Zeiher A M, Vehreschild M and Nagel E. Outcomes of Cardiovascular Magnetic Resonance Imaging in Patients Recently Recovered From Coronavirus Disease 2019 (COVID-19). JAMA cardiology. 2020. They are also critical downstream components of growth factor receptor (GFR) signaling cascades, which drive phosphorylation of viral proteins upon SARS-CoV-2 infection. See, van der Heijden R A, Bijzet J, Meijers W C, Yakala G K, Kleemann R, Nguyen T Q, de Boer R A, Schalkwijk C G, Hazenberg B P, Tietge U J and Heeringa P. Obesity-induced chronic inflammation in high fat diet challenged C57BL/6J mice is associated with acceleration of age-dependent renal amyloidosis. Sci Rep. 2015; 5:16474. Therefore, this class of enzymes has been proposed as a druggable target for prevention and treatment of SARS-CoV-2 infection. Indeed, inhibition of class I or class III PI3K (SEQ ID NO: 8) prevents viral replication, see van der Heijden R A Id. and Qin Q, Qu C, Niu T, Zang H, Qi L, Lyu L, Wang X, Nagarkatti M, Nagarkatti P, Janicki J S, Wang X L and Cui T. Nrf2-Mediated Cardiac Maladaptive Remodeling and Dysfunction in a Setting of Autophagy Insufficiency. Hypertension. 2016; 67:107-17, probably through distinct mechanistic actions on different stages of SARS-CoV-2 viral life cycle. In a distinct mechanism, our data shows that the Spike protein alone can dysregulate expression of various PI3Ks (SEQ ID NO: 8) in host cells including upregulation of class I PIK3CA (SEQ ID NO: 18), PIK3CD (SEQ ID NO: 19), and PIK3R3 (SEQ ID NO: 20), but downregulation of class 3 PIK3C3 (SEQ ID NO: 22), which is required for autophagosome and lysosome fusion. See, Gao Z, Daquinag A C, Su F, Snyder B and Kolonin M G. PDGFRalpha/PDGFRbeta signaling balance modulates progenitor cell differentiation into white and beige adipocytes. Development. 2018; 145.

In addition, pan-PI3K (SEQ ID NO: 8) inhibitor wortmannin shows a potent inhibition of the Spike-protein exaggerated PA-induced lipotoxicity, suggesting that increasing autophagosome formation while decreasing autophagosome fusion with lysosomes thereby leading to accumulation of autophagosomes may be a cause of Spike protein-exaggerated lipotoxicity. Therefore, targeting of PI3K (SEQ ID NO: 8) can be potentially beneficial for those COVID-19 patients with a metabolic precondition of hyperlipidemia.

The transcription factor Nrf2 (SEQ ID NO: 1) controls the basal and induced expression of more than 1000 genes in cells that can be clustered into several groups with distinct functions, such as antioxidative defense, detoxification, protein degradation, and iron and lipid metabolism. See, Mizunoe Y. Id. Thus, the functions of Nrf2 (SEQ ID NO: 1) spread rather broadly from antioxidative defense to protein quality control and metabolism regulation. Studies have demonstrated that Nrf2 (SEQ ID NO: 1) is required for cardiac adaptation when cardiac autophagy is intact; however, it operates a pathological programme to exacerbate maladaptive cardiac remodeling and dysfunction when myocardial autophagy is inhibited in the settings of sustained pressure overload, see Marcelin G, Ferreira A, Liu Y, Atlan M, Aron-Wisnewsky J, Pelloux V, Botbol Y, Ambrosini M, Fradet M, Rouault C, Henegar C, Hulot J S, Poitou C, Torcivia A, Nail-Barthelemy R, Bichet J C, Gautier E L and Clement K. A PDGFRalpha-Mediated Switch toward CD9(high) Adipocyte Progenitors Controls Obesity-Induced Adipose Tissue Fibrosis. Cell Metab. 2017; 25:673-685, and chronic type 1 diabetes, see Bao L. Id. Notably, chronic obesity, a pre-type 2 diabetic setting, results in inhibition of myocardial autophagy, thereby leading to cardiac pathological remodeling and dysfunction. See, Kusminski C M, Park J and Scherer P E. MitoNEET-mediated effects on browning of white adipose tissue. Nat Commun. 2014; 5:3962 and Wu H and Ballantyne C M. Skeletal muscle inflammation and insulin resistance in obesity. J Clin Invest. 2017; 127:43-54. Here, the protein level of Nrf2 (SEQ ID NO: 1) is upregulated in the Spike cells in response to PA treatment. Furthermore, TRG, a Nrf2 (SEQ ID NO: 1) inhibitor, can attenuate Spike-protein-exaggerated PA-induced necrosis in the cell with impaired autophagy. Collectively, it is reasonable to posit a central role of Nrf2 (SEQ ID NO: 1) in PA-induced Spike protein-exaggerated lipotoxicity in host cells. However, the detailed pathological mechanism and molecular interactions mediated by impaired Nrf2 (SEQ ID NO: 1) pathways need further validation.

Herein, the concentrations of PA the inventors used are supraphysiological. The serum average levels of PA range 160 M in normal subjects and 220 M in obese, or 160 M in nondiabetic subjects to 280 M in diabetic subjects after fast. See, Delorme-Axford E and Klionsky D J. Highlights in the fight against COVID-19: does autophagy play a role in SARS-CoV-2 infection?Autophagy. 2020; 16:2123-2127; Randhawa P K, Scanlon K, Rappaport J and Gupta M K. Modulation of Autophagy by SARS-CoV-2: A Potential Threat for Cardiovascular System. Front Physiol. 2020; 11:611275; Mathis B J, Lai Y, Qu C, Janicki J S and Cui T. CYLD-mediated signaling and diseases. Current drug targets. 2015; 16:284-94; and Sun S C. CYLD: a tumor suppressor deubiquitinase regulating NF-kappaB activation and diverse biological processes. Cell Death Differ. 2010; 17:25-34. observed a modest or mild effect at 500 M and 250 M after 72 h treatment of PA on the Spike cells, indicating that the Spike cells may be sensitive to a physiologically relevant PA concentration after a long-term treatment (>72 h).

The potential effects of other types of fatty acids or lipid species on the Spike cells are not evaluated. In addition, the types of altered lipids or lipid metabolites caused by the Spike protein have not been identified. Additional pan-caspase inhibitors are needed to exclude the Caspase-dependent mediated apoptosis in the Spike cells after PA treatment. The functional domain(s) of the Spike protein-mediated lipotoxicity and the subcellular locations of expressed Spike are not defined, which can be assessed using a series of constructs to express various truncated Spike mutations. The HEK has very low levels of endogenous ACE2 (SEQ ID NO: 2) and TMPRSS2 (SEQ ID NO: 26). Therefore, it is unlikely a mechanistic involvement of the signaling pathway associated with a secretion of Spike and binding to the surface ACE2 (SEQ ID NO: 2) receptor. Instead, the current disclosure demonstrates that the Spike-induced intracellular pathology is largely related to lipotoxicity and impaired autophagy. Disposition of lipid droplets in the Spike cells shows a predominant cell membrane distribution, indicating that the Spike-mediated lipotoxicity is associated with cell membranes. However, the detailed mechanisms and precise subcellular locations of lipid droplets are yet to be determined and need further investigations. This disclosure is the initial step to reveal the pathological role of the Spike mediated lipotoxicity that is related to autophagy/necroptosis/ferroptosis, indicating a central role of Nrf2 (SEQ ID NO: 1). However, the detailed molecular mechanism underlying the Spike protein-induced lipidomic dysregulation is unknown. The disclosure's data indicates that Nrf2 (SEQ ID NO: 1) may play a central role in the transcriptional level for this pathological process. However, this needs further elucidation and validation using loss-of-function and gain-of-function approaches. The current emerging COVID variants, omicron strains, present multiple mutations in the Spike protein; whether these omicron versions of Spike protein variants can enhance or attenuate their functions in lipid metabolic alteration as compared with the alpha version of the Spike protein is unknown. Lastly, the Spike protein-induced impairments in both autophagic and lipid metabolic pathways in host cells are evident. However, whether autophagic impairment is a consequence of or in parallel to lipid metabolic impairments is unknown. Most likely, autophagic impairment is intertangled with lipidomic alterations not only as a result of but also an independent factor for the deterioration in response to lipotoxicity.

In conclusion, this disclosure has demonstrated that the Spike protein can cause lipid deposition and impair lipid metabolic and autophagic pathways in host cells, ultimately leading to increased susceptibility to lipotoxicity via ferroptosis. The Spike protein-enhanced lipotoxicity can be suppressed by the Nrf2 (SEQ ID NO: 1) inhibitor TRG, indicating a central role of Nrf2 (SEQ ID NO: 1) in COVID-19-associated cardiac complications involving obesity.

### Spike-EVs Induces Mitochondria Stress in Endothelial Cells (ECs):

Mitochondrial dysfunction-induced chronic inflammation is closely associated with heart failures. See, Mann, D. L. Innate immunity and the failing heart: the cytokine hypothesis revisited. Circ. Res. 116, 1254-1268, doi:10.1161/CIRCRESAHA.116.302317 (2015); Zhang, Q. et al. Circulating mitochondrial DAMPs cause inflammatory responses to injury. Nature 464, 104-107, doi:10.1038/nature08780 (2010); and Dela Cruz, C. S. & Kang, M. J. Mitochondrial dysfunction and damage associated molecular patterns (DAMPs) in chronic inflammatory diseases. Mitochondrion 41, 37-44, doi:10.1016/j.mito.2017.12.001 (2018). The spike protein alone can induce cardiovascular pathologies. See, Avolio, E. et al. The SARS-CoV-2 Spike protein disrupts human cardiac pericytes function through CD147 receptor-mediated signaling: a potential non-infective mechanism of COVID-19 microvascular disease. Clin. Sci. (Lond.) 135, 2667-2689, doi:10.1042/CS20210735 (2021); Lei, Y. et al. SARS-CoV-2 Spike Protein Impairs Endothelial Function via Downregulation of ACE 2. Circ Res 128, 1323-1326, doi:10.1161/CIRCRESAHA.121.318902 (2021); and DeOre, B. J., Tran, K. A., Andrews, A. M., Ramirez, S. H. & Galie, P. A. SARS-CoV-2 Spike Protein Disrupts Blood-Brain Barrier Integrity via RhoA Activation. J Neuroimmune Pharmacol 16, 722-728, doi:10.1007/s11481-021-10029-0 (2021). Circulating spike-extracellular vesicles (EVs) are found to be present in both acute COVID-19 and PASC patients. See, Vaughn Craddock et al. Persistent Presence of Spike protein and Viral RNA in the Circulation of Individuals with Post-Acute Sequelae of COVID-19. medRxiv, doi.org/10.1101/2022.08.07.22278520 (2022). Mitochondrial alterations are found in COVID-19 patients. See, Gibellini, L. et al. Altered bioenergetics and mitochondrial dysfunction of monocytes in patients with COVID-19 pneumonia. EMBO Mol Med 12, e13001, doi:10.15252/emmm.202013001 (2020). The inventors posited that spike-EVs could induce mitochondrial dysfunctions in ECs.

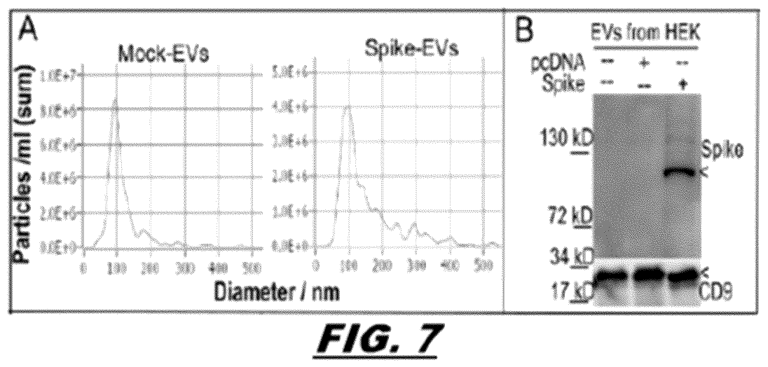

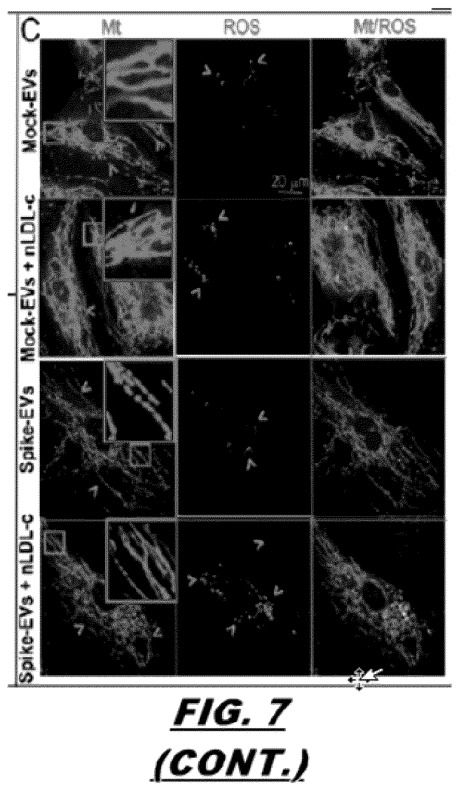

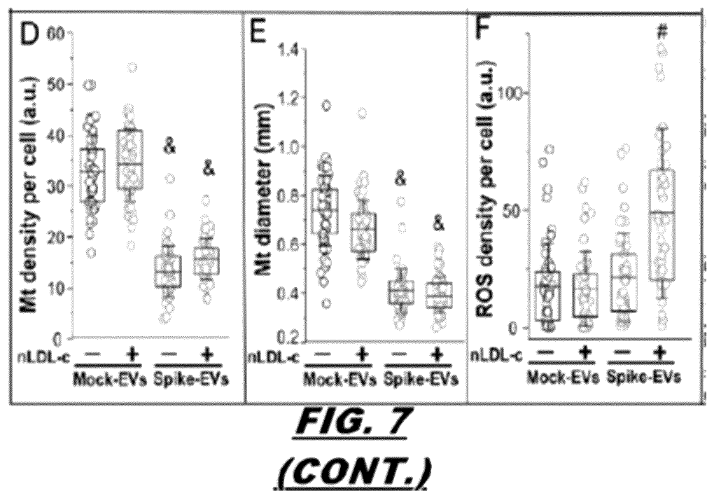

To test this, the inventors isolated spike-EVs and EVs without a spike protein (Mock-EVs) from our established HEK cell line with spike protein (Wuhan variant) stable-expression and the HEK cells with integrated pcDNA vector only, respectively. Nguyen, V. et al. The Spike Protein of SARS-CoV-2 Impairs Lipid Metabolism and Increases Susceptibility to Lipotoxicity: Implication for a Role of Nrf2 (SEQ ID NO: 1). Cells 11, doi:10.3390/cells11121916 (2022). These cell lines released EVs into the culture medium constantly. The number and size of the EVs were characterized using nanoparticle tracking analysis (NTA) (ZetaView® QUATT). See, FIG. 7 at A. The mean sizes of the mock-EVs and spike-EVs were about 96.3 nm and 112.8 nm, respectively. The spike protein was detected in the spike-EVs but not both controls. See, FIG. 7 at B. It was about 1.27×10⁻⁸ μg spike protein/EV particle determined using an ELISA assay. Human dermal microvascular ECs (hDMVECs) were cultured in a medium containing 5% EV-depleted FBS (final LDL-c levels in the medium: ˜10 μg/ml) with or without addition of 100 μg/ml human native LDL cholesterol (nLDL-c). The mock-EVs or spike-EVs (5-7×10⁹ particles) were added into cells for 48 hours. The spike-EVs caused decreases in mitochondrial density per cell and their diameters as compared with the mock-EVs; these effects were nLDL-c independent. See FIG. 7 at C-E. The spike-EVs with nLDL-c significantly increased cellular ROS levels; but this effect was not observed with either spike-EVs alone or mock-EVs+nLDL-c. See FIG. 7 at C, F. FIG. 7 shows ig. 1. Spike-EVs induce mitochondria (Mt) stress in hDMVECs. (A) Size and quantity of fabricated mock-EVs and spike-EVs determined by NTA. (B) Spike protein was detected in the fabricated spike-EVs from HEK cells using western blot. An anti-S2 subunit antibody was used. (C) MitoTracker (Red CMXRos, Cellsignal) and CellRox Green (ThermoFisher) were used to stain Mt (yellow arrowheads) and detect cellular ROS levels (green arrowheads), respectively. The green insert is a higher magnification of the region in yellow box in each image showing Mt morphology. (D) Mt density per cell (arbitrary unit, a.u.). (E) Mt diameters (μm); &, p<0.0001 compared to both mock-EVs treatment groups in both (D) & (E). (F) Quantity of cellular ROS signal per cell (a.u.) among various groups; #, p<0.0001 compared to other groups.

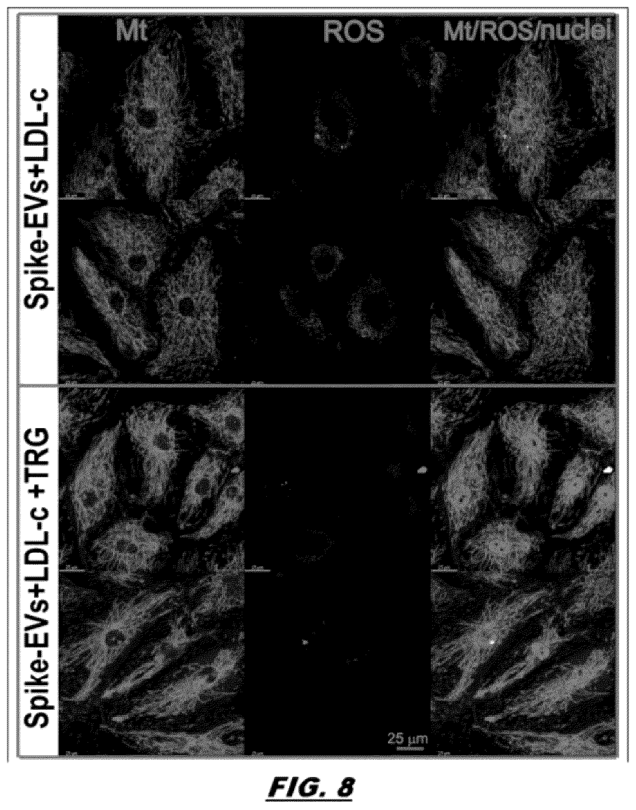

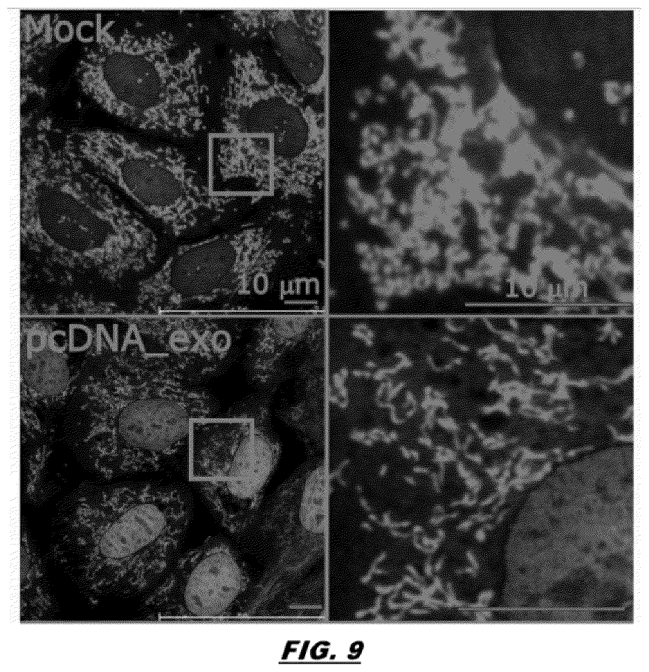

Spike-EVs plus LDL-c-induced mitochondria stress and increases in ROS could be reversed by TRG: The inventors next tested whether TRG could restore the mitochondria morphology and suppress the ROS induced by Spike-EVs plus LDL-c in ECs. The HDMVECs were treated by Spike-EVs and LDL-c with or without addition of TRG. The inventors found TRG could restore the decreased density and diameter of mitochondria caused by Spike-EVs plus LDL-c. The cellular ROS levels were also attenuated with the treatment of TRG. See, FIG. 8. FIG. 8 shows Spike-EVs plus LDL-c-induced mitochondria stress and increases in ROS could be reversed by TRG in hDMVECs. MitoTracker (Red CMXRos, Cellsignal) and CellRox Green (ThermoFisher) were used to stain Mt and detect cellular ROS levels, respectively. The hDMVECs were treated with spike-EVs plus LDL-c with or without TRG.

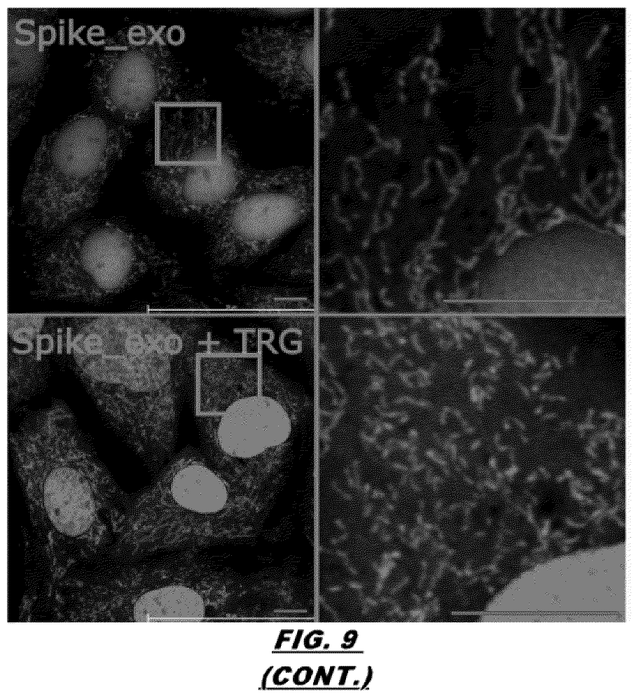

FIG. 9 shows living mitochondria were stained by MitoTracker and nucleus were stained by Hoechst 33342. The scale bar is 10 μm. The right panels are higher magnifications of the corresponding areas in the pink boxes from images in the left panels. The H9C2 cells were treated without exosomes (mock), or with pcDNA-exosomes (pcDNA-exo), spike-exosomes (spike-exo), or spike-exo plus TRG.

### Methods

(1) preparation of spike-protein containing exosomes: HEK293 cells were cultured in a DMEM medium and transected by a plasmid expression of the spike protein (pcDNA-spike) or empty vector pcDNA3.1 as a control. The stable cell lines with consecutive expressions of the spike protein were obtained. The exosomes were isolated and purified from the culture medium of pcDNA-spike or control pcDNA stable cell lines, which were labeled as spike-exo or pcDNA-exo, respectively. The expression of spike protein in the spike-exo was verified using a Western blot.

(2) cardiomyocyte-like cells H9C2 were cultured in a DMEM complete medium containing 10% FBS, reaching to a confluence about 50%-60%. The cells were switched into a DMEM with 10% exosomes-free FBS. The pcDNA-exo or spike-exo were added into the medium (equivalent to 150 μg exosomal proteins in 1 ml medium) with or without TRG (125 μM). The medium was changed every other day and treatments were given for a total of 5 days. The cells were then stained using a living mitochondrial tracker fluorescent dye (MitoTracker, ThermoFisher). The images were acquired under a confocal microscope.

Results: The mitochondria showed oval or globular shapes in the H9C2 cells without any exosome treatment (the mock group). The mitochondria exhibited a tubular shape in a small size after being treated by pcDNA-exosomes (pcDNA-exo). The spike-exosomes (spike-exo) caused a dramatic mitochondrial fusion (thin and elongated shape) in response to cellular stress with interconnected network OXPHOS. TRG can reverse the spike-exo induced mitochondrial fusion, showing mitochondria in small and tubular morphologies. This data further supports our previous animal study, demonstrating that cardiac mitochondrial dysfunction is the crucial and long-term pathological factor underlying spike-protein induced cardiomyopathy, which can be mitigated by TRG.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### Sequence Listing Xml Incorporation Statement

The herewith provided Sequence Listing XML is hereby incorporated by reference. The name of the XML file is USC1642.xml with a date of creation of Feb. 13, 2024 and a size of 39 kilobytes.

SEQUENCE LISTING-USC 2033101.0000449

| | |
|---|---|
| <110> | University of South Carolina |
| <120> | TRIGONELLINE MITIGATES CARDIOMYOPATHY CAUSED BY SARS- |
| | COV-2 SPIKE PROTEIN UNDER OBESITY |
| <130> | 2033101.0000449 |
| <140> | Unknown Current Application Number |
| <141> | 02/13/2024 |
| <150> | U.S. 63/495,823 |
| <151> | 2023-04-13 |
| <160> | 26 |
| <170> | WIPO Standard ST.26 |

Protein: Nrf2

| | |
|---|---|
| <210> | 1 |
| <211> | 605 |
| <212> | PRT |
| <213> | Homo sapiens |
| <221> | WIPO Standard ST.26 |
| <222> | 1...605 |
| <400> | 1 |

MMDLELPPPGLPSQQDMDLIDILWRQDIDLGVSREVFDFSQRRKEYELEKQK
KLEKERQEQLQKEQEKAFFAQLQLDEETGEFLPIQPAQHIQSETSGSANYSQVAHIPKSD
ALYFDDCMQLLAQTFPFVDDNEVSSATFQSLVPDIPGHIESPVFIATNQAQSPETSVAQV
APVDLDGMQQDIEQVWEELLSIPELQCLNIENDKLVETTMVPSPEAKLTEVDNYHFYSSI
PSMEKEVGNCSPHFLNAFEDSFSSILSTEDPNQLTVNSLNSDATVNTDFGDEFYSAFIAEP
SISNSMPSPATLSHSLSELLNGPIDVSDLSLCKAFNQNHPESTAEFNDSDSGISLNTSPSVA
SPEHSVESSSYGDTLLGLSDSEVEELDSAPGSVKQNGPKTPVHSSGDMVQPLSPSQGQST
HVHDAQCENTPEKELPVSPGHRKTPFTKDKHSSRLEAHLTRDELRAKALHIPFPVEKIIN
LPVVDFNEMMSKEQFNEAQLALIRDIRRRGKNKVAAQNCRKRKLENIVELEQDLDHLK
DEKEKLLKEKGENDKSLHLLKKQLSTLYLEVFSMLRDEDGKPYSPSEYSLQQTRDGNVF
LVPKSKKPDVKKN
MetMetAspLeuGluLeuProProProGlyLeuProSerGlnGlnAspMetAspLeuIleAspIleLeu
TrpArgGlnAspIleAspLeuGlyValSerArgGluValPheAspPheSerGlnArgArgLysGluTyrGluLeuGlu
LysGlnLysLysLeuGluLysGluArgGlnGluGlnLeuGlnLysGluGlnGluLysAlaPhePheAlaGlnLeu
GlnLeuAspGluGluThrGlyGluPheLeuProIleGlnProAlaGlnHisIleGlnSerGluThrSerGlySerAlaAsn
TyrSerGlnValAlaHisIleProLysSerAspAlaLeuTyrPheAspAspCysMetGlnLeuLeuAlaGlnThrPhePro
PheValAspAspAsnGluValSerSerAlaThrPheGlnSerLeuValProAspIleProGlyHisIleGluSerProVal
PheIleAlaThrAsnGlnAlaGlnSerProGluThrSerValAlaGlnValAlaProValAspLeuAspGlyMetGln
GlnAspIleGluGlnValTrpGluGluLeuLeuSerIleProGluLeuGlnCysLeuAsnIleGluAsnAspLysLeuVal
GluThrThrMetValProSerProGluAlaLysLeuThrGluValAspAsnTyrHisPheTyrSerSerIleProSerMet
GluLysGluValGlyAsnCysSerProHisPheLeuAsnAlaPheGluAspSerPheSerSerIleLeuSerThrGlu
AspProAsnGlnLeuThrValAsnSerLeuAsnSerAspAlaThrValAsnThrAspPheGlyAspGluPheTyrSer
AlaPheIleAlaGluProSerIleSerAsnSerMetProSerProAlaThrLeuSerHisSerLeuSerGluLeuLeuAsnGly
ProIleAspValSerAspLeuSerLeuCysLysAlaPheAsnGlnAsnHisProGluSerThrAlaGluPheAsnAsp
SerAspSerGlyIleSerLeuAsnThrSerProSerValAlaSerProGluHisSerValGluSerSerSerTyrGlyAsp
ThrLeuLeuGlyLeuSerAspSerGluValGluGluLeuAspSerAlaProGlySerValLysGlnAsnGlyProLysThr
ProValHisSerSerGlyAspMetValGlnProLeuSerProSerGlnGlyGlnSerThrHisValHisAspAlaGln
CysGluAsnThrProGluLysGluLeuProValSerProGlyHisArgLysThrProPheThrLysAspLysHisSerSer
ArgLeuGluAlaHisLeuThrArgAspGluLeuArgAlaLysAlaLeuHisIleProPheProValGluLysIleIle
AsnLeuProValValAspPheAsnGluMetMetSerLysGluGlnPheAsnGluAlaGlnLeuAlaLeuIleArgAspIle
ArgArgArgGlyLysAsnLysValAlaAlaGlnAsnCysArgLysArgLysLeuGluAsnIleValGluLeuGlu
GlnAspLeuAspHisLeuLysAspGluLysGluLysLeuLeuLysGluLysGlyGluAsnAspLysSerLeuHisLeu
LeuLysLysGlnLeuSerThrLeuTyrLeuGluValPheSerMetLeuArgAspGluAspGlyLysProTyrSerPro
SerGluTyrSerLeuGlnGlnThrArgAspGlyAsnValPheLeuValProLysSerLysLysProAspValLysLys
Asn
Protein: ACE2

| | |
|---|---|
| <210> | 2 |
| <211> | 805 |
| <212> | PRT |
| <213> | Homo sapiens |
| <221> | WIPO Standard ST.26 |
| <222> | 1...805 |
| <400> | 2 |

MSSSSWLLLSLVAVTAAQSTIEEQAKTFLDKFNHEAEDLFYQSSLASWNYNT
NITEENVQNMNNAGDKWSAFLKEQSTLAQMYPLQEIQNLTVKLQLQALQQNGSSVLSE
DKSKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLIED
VEHTFEEIKPLYEHLHAYVRAKLMNAYPSYISPIGCLPAHLLGDMWGRFWTNLYSLTVP
FGQKPNIDVTDAMVDQAWDAQRIFKEAEKFFVSVGLPNMTQGFWENSMLTDPGNVQK
AVCHPTAWDLGKGDFRILMCTKVTMDDFLTAHHEMGHIQYDMAYAAQPFLLRNGANE
GFHEAVGEIMSLSAATPKHLKSIGLLSPDFQEDNETEINFLLKQALTIVGTLPFTYMLEKW
RWMVFKGEIPKDQWMKKWWEMKREIVGVVEPVPHDETYCDPASLFHVSNDYSFIRYY
TRTLYQFQFQEALCQAAKHEGPLHKCDISNSTEAGQKLFNMLRLGKSEPWTLALENVV
GAKNMNVRPLLNYFEPLFTWLKDQNKNSFVGWSTDWSPYADQSIKVRISLKSALGDKA
YEWNDNEMYLFRSSVAYAMRQYFLKVKNQMILFGEEDVRVANLKPRISFNFFVTAPKN
VSDIIPRTEVEKAIRMSRSRINDAFRLNDNSLEFLGIQPTLGPPNQPPVSIWLIVFGVVMGV
IVVGIVILIFTGIRDRKKKNKARSGENPYASIDISKGENNPGFQNTDDVQTSF
MetSerSerSerSerTrpLeuLeuLeuSerLeuValAlaValThrAlaAlaGlnSerThrIleGluGluGln
AlaLysThrPheLeuAspLysPheAsnHisGluAlaGluAspLeuPheTyrGlnSerSerLeuAlaSerTrpAsnTyr
AsnThrAsnIleThrGluGluAsnValGlnAsnMetAsnAsnAlaGlyAspLysTrpSerAlaPheLeuLysGluGln
SerThrLeuAlaGlnMetTyrProLeuGlnGluIleGlnAsnLeuThrValLysLeuGlnLeuGlnAlaLeuGlnGln
AsnGlySerSerValLeuSerGluAspLysSerLysArgLeuAsnThrIleLeuAsnThrMetSerThrIleTyrSer
ThrGlyLysValCysAsnProAspAsnProGlnGluCysLeuLeuLeuGluProGlyLeuAsnGluIleMetAlaAsn
SerLeuAspTyrAsnGluArgLeuTrpAlaTrpGluSerTrpArgSerGluValGlyLysGlnLeuArgProLeuTyr
GluGluTyrValValLeuLysAsnGluMetAlaArgAlaAsnHisTyrGluAspTyrGlyAspTyrTrpArgGlyAspT
yrGluValAsnGlyValAspGlyTyrAspTyrSerArgGlyGlnLeuIleGluAspValGluHisThrPheGluGluIle
LysProLeuTyrGluHisLeuHisAlaTyrValArgAlaLysLeuMetAsnAlaTyrProSerTyrIleSerProIleGly
CysLeuProAlaHisLeuLeuGlyAspMetTrpGlyArgPheTrpThrAsnLeuTyrSerLeuThrValProPheGly
GlnLysProAsnIleAspValThrAspAlaMetValAspGlnAlaTrpAspAlaGlnArgIlePheLysGluAlaGlu
LysPhePheValSerValGlyLeuProAsnMetThrGlnGlyPheTrpGluAsnSerMetLeuThrAspProGlyAsn
ValGlnLysAlaValCysHisProThrAlaTrpAspLeuGlyLysGlyAspPheArgIleLeuMetCysThrLysVal
ThrMetAspAspPheLeuThrAlaHisHisGluMetGlyHisIleGlnTyrAspMetAlaTyrAlaAlaGlnProPhe
LeuLeuArgAsnGlyAlaAsnGluGlyPheHisGluAlaValGlyGluIleMetSerLeuSerAlaAlaThrProLysHis
LeuLysSerIleGlyLeuLeuSerProAspPheGlnGluAspAsnGluThrGluIleAsnPheLeuLeuLysGlnAla
LeuThrIleValGlyThrLeuProPheThrTyrMetLeuGluLysTrpArgTrpMetValPheLysGlyGluIleProLys
AspGlnTrpMetLysLysTrpTrpGluMetLysArgGluIleValGlyValValGluProValProHisAspGluThr
TyrCysAspProAlaSerLeuPheHisValSerAsnAspTyrSerPheIleArgTyrTyrThrArgThrLeuTyrGlnPhe
GlnPheGlnGluAlaLeuCysGlnAlaAlaLysHisGluGlyProLeuHisLysCysAspIleSerAsnSerThrGlu
AlaGlyGlnLysLeuPheAsnMetLeuArgLeuGlyLysSerGluProTrpThrLeuAlaLeuGluAsnValValGly
AlaLysAsnMetAsnValArgProLeuLeuAsnTyrPheGluProLeuPheThrTrpLeuLysAspGlnAsnLys
AsnSerPheValGlyTrpSerThrAspTrpSerProTyrAlaAspGlnSerIleLysValArgIleSerLeuLysSerAla
LeuGlyAspLysAlaTyrGluTrpAsnAspAsnGluMetTyrLeuPheArgSerSerValAlaTyrAlaMetArgGln
TyrPheLeuLysValLysAsnGlnMetIleLeuPheGlyGluGluAspValArgValAlaAsnLeuLysProArgIle
SerPheAsnPhePheValThrAlaProLysAsnValSerAspIleIleProArgThrGluValGluLysAlaIleArgMet
SerArgSerArgIleAsnAspAlaPheArgLeuAsnAspAsnSerLeuGluPheLeuGlyIleGlnProThrLeuGlyPro
ProAsnGlnProProValSerIleTrpLeuIleValPheGlyValValMetGlyValIleValValGlyIleValIleLeuIle
PheThrGlyIleArgAspArgLysLysLysAsnLysAlaArgSerGlyGluAsnProTyrAlaSerIleAspIleSerLys
GlyGluAsnAsnProGlyPheGlnAsnThrAspAspValGlnThrSerPhe
Protein: ASCL4

| | |
|---|---|
| <210> | 3 |
| <211> | 172 |
| <212> | PRT |
| <213> | Homo sapiens |
| <221> | WIPO Standard ST.26 |
| <222> | 1...172 |
| <400> | 3 |

METRKPAERLALPYSLRTAPLGVPGTLPGLPRRDPLRVALRLDAACWEWAR
SGCARGWQYLPVPLDSAFEPAFLRKRNERERQRVRCVNEGYARLRDHLPRELADKRLS
KVETLRAAIDYIKHLQELLERQAWGLEGAAGAVPQRRAECNSDGESKASSAPSPSSEPE
EGGS
MetGluThrArgLysProAlaGluArgLeuAlaLeuProTyrSerLeuArgThrAlaProLeuGlyVal
ProGlyThrLeuProGlyLeuProArgArgAspProLeuArgValAlaLeuArgLeuAspAlaAlaCysTrpGluTrp
AlaArgSerGlyCysAlaArgGlyTrpGlnTyrLeuProValProLeuAspSerAlaPheGluProAlaPheLeuArg
LysArgAsnGluArgGluArgGlnArgValArgCysValAsnGluGlyTyrAlaArgLeuArgAspHisLeuPro
ArgGluLeuAlaAspLysArgLeuSerLysValGluThrLeuArgAlaAlaIleAspTyrIleLysHisLeuGlnGluLeu
LeuGluArgGlnAlaTrpGlyLeuGluGlyAlaAlaGlyAlaValProGlnArgArgAlaGluCysAsnSerAspGly
GluSerLysAlaSerSerAlaProSerProSerSerGluProGluGluGlyGlySer
Protein: Fth1

| | |
|---|---|
| <210> | 4 |
| <211> | 183 |
| <212> | PRT |
| <213> | Homo sapiens |
| <221> | WIPO Standard ST.26 |
| <222> | 1...183 |
| <400> | 4 |

MTTASTSQVRQNYHQDSEAAINRQINLELYASYVYLSMSYYFDRDDVALKN
FAKYFLHQSHEEREHAEKLMKLQNQRGGRIFLQDIKKPDCDDWESGLNAMECALHLEK
NVNQSLLELHKLATDKNDPHLCDFIETHYLNEQVKAIKELGDHVTNLRKMGAPESGLA
EYLFDKHTLGDSDNES
MetThrThrAlaSerThrSerGlnValArgGlnAsnTyrHisGlnAspSerGluAlaAlaIleAsnArg
GlnIleAsnLeuGluLeuTyrAlaSerTyrValTyrLeuSerMetSerTyrTyrPheAspArgAspAspValAlaLeu
LysAsnPheAlaLysTyrPheLeuHisGlnSerHisGluGluArgGluHisAlaGluLysLeuMetLysLeuGlnAsn
GlnArgGlyGlyArgIlePheLeuGlnAspIleLysLysProAspCysAspAspTrpGluSerGlyLeuAsnAlaMet
GluCysAlaLeuHisLeuGluLysAsnValAsnGlnSerLeuLeuGluLeuHisLysLeuAlaThrAspLysAsnAsp
ProHisLeuCysAspPheIleGluThrHisTyrLeuAsnGluGlnValLysAlaIleLysGluLeuGlyAspHisValThr
AsnLeuArgLysMetGlyAlaProGluSerGlyLeuAlaGluTyrLeuPheAspLysHisThrLeuGlyAspSer
AspAsnGluSer
Protein: PTGS2

| | |
|---|---|
| <210> | 5 |
| <211> | 604 |
| <212> | PRT |
| <213> | Homo sapiens |
| <221> | WIPO Standard ST.26 |
| <222> | 1...604 |
| <400> | 5 |

MLARALLLCAVLALSHTANPCCSHPCQNRGVCMSVGFDQYKCDCTRTGFYG
ENCSTPEFLTRIKLFLKPTPNTVHYILTHFKGFWNVVNNIPFLRNAIMSYVLTSRSHLIDSP
PTYNADYGYKSWEAFSNLSYYTRALPPVPDDCPTPLGVKGKKQLPDSNEIVEKLLLRRK
FIPDPQGSNMMFAFFAQHFTHQFFKTDHKRGPAFTNGLGHGVDLNHIYGETLARQRKLR
LFKDGKMKYQIIDGEMYPPTVKDTQAEMIYPPQVPEHLRFAVGQEVFGLVPGLMMYAT
IWLREHNRVCDVLKQEHPEWGDEQLFQTSRLILIGETIKIVIEDYVQHLSGYHFKLKFDPE
LLFNKQFQYQNRIAAEFNTLYHWHPLLPDTFQIHDQKYNYQQFIYNNSILLEHGITQFVE
SFTRQIAGRVAGGRNVPPAVQKVSQASIDQSRQMKYQSFNEYRKRFMLKPYESFEELTG
EKEMSAELEALYGDIDAVELYPALLVEKPRPDAIFGETMVEVGAPFSLKGLMGNVICSP
AYWKPSTFGGEVGFQIINTASIQSLICNNVKGCPFTSFSVPDPELIKTVTINASSSRSGLDDI
NPTVLLKERSTEL
MetLeuAlaArgAlaLeuLeuLeuCysAlaValLeuAlaLeuSerHisThrAlaAsnProCysCysSer
HisProCysGlnAsnArgGlyValCysMetSerValGlyPheAspGlnTyrLysCysAspCysThrArgThrGly
PheTyrGlyGluAsnCysSerThrProGluPheLeuThrArgIleLysLeuPheLeuLysProThrProAsnThrValHis
TyrIleLeuThrHisPheLysGlyPheTrpAsnValValAsnAsnIleProPheLeuArgAsnAlaIleMetSerTyr
ValLeuThrSerArgSerHisLeuIleAspSerProProThrTyrAsnAlaAspTyrGlyTyrLysSerTrpGluAlaPhe
SerAsnLeuSerTyrTyrThrArgAlaLeuProProValProAspAspCysProThrProLeuGlyValLysGlyLysLys
GlnLeuProAspSerAsnGluIleValGluLysLeuLeuLeuArgArgLysPheIleProAspProGlnGlySerAsn
MetMetPheAlaPhePheAlaGlnHisPheThrHisGlnPhePheLysThrAspHisLysArgGlyProAlaPheThr
AsnGlyLeuGlyHisGlyValAspLeuAsnHisIleTyrGlyGluThrLeuAlaArgGlnArgLysLeuArgLeuPhe
LysAspGlyLysMetLysTyrGlnIleIleAspGlyGluMetTyrProProThrValLysAspThrGlnAlaGluMet
IleTyrProProGlnValProGluHisLeuArgPheAlaValGlyGlnGluValPheGlyLeuValProGlyLeuMetMet
TyrAlaThrIleTrpLeuArgGluHisAsnArgValCysAspValLeuLysGlnGluHisProGluTrpGlyAspGlu
GlnLeuPheGlnThrSerArgLeuIleLeuIleGlyGluThrIleLysIleValIleGluAspTyrValGlnHisLeuSer
GlyTyrHisPheLysLeuLysPheAspProGluLeuLeuPheAsnLysGlnPheGlnTyrGlnAsnArgIleAlaAla
GluPheAsnThrLeuTyrHisTrpHisProLeuLeuProAspThrPheGlnIleHisAspGlnLysTyrAsnTyrGlnGln
PheIleTyrAsnAsnSerIleLeuLeuGluHisGlyIleThrGlnPheValGluSerPheThrArgGlnIleAlaGlyArg
ValAlaGlyGlyArgAsnValProProAlaValGlnLysValSerGlnAlaSerIleAspGlnSerArgGlnMetLys
TyrGlnSerPheAsnGluTyrArgLysArgPheMetLeuLysProTyrGluSerPheGluGluLeuThrGlyGluLys
GluMetSerAlaGluLeuGluAlaLeuTyrGlyAspIleAspAlaValGluLeuTyrProAlaLeuLeuValGluLysPro
ArgProAspAlaIlePheGlyGluThrMetValGluValGlyAlaProPheSerLeuLysGlyLeuMetGlyAsnVal
IleCysSerProAlaTyrTrpLysProSerThrPheGlyGlyGluValGlyPheGlnIleIleAsnThrAlaSerIleGlnSer
LeuIleCysAsnAsnValLysGlyCysProPheThrSerPheSerValProAspProGluLeuIleLysThrValThrIle
AsnAlaSerSerSerArgSerGlyLeuAspAspIleAsnProThrValLeuLeuLysGluArgSerThrGluLeu
Protein: LC3

| | |
|---|---|
| <210> | 6 |
| <211> | 125 |
| <212> | PRT |
| <213> | Homo sapiens |
| <221> | WIPO Standard ST.26 |
| <222> | 1...125 |
| <400> | 6 |

MPSEKTFKQRRTFEQRVEDVRLIREQHPTKIPVIIERYKGEKQLPVLDKTKFLV
PDHVNMSELIKIIRRRLQLNANQAFFLLVNGHSMVSVSTPISEVYESEKDEDGFLYMVYA
SQETFGMKLSV
MetProSerGluLysThrPheLysGlnArgArgThrPheGluGlnArgValGluAspValArgLeuIle
ArgGluGlnHisProThrLysIleProValIleIleGluArgTyrLysGlyGluLysGlnLeuProValLeuAspLysThr
LysPheLeuValProAspHisValAsnMetSerGluLeuIleLysIleIleArgArgArgLeuGlnLeuAsnAlaAsn
GlnAlaPhePheLeuLeuValAsnGlyHisSerMetValSerValSerThrProIleSerGluValTyrGluSerGluLys
AspGluAspGlyPheLeuTyrMetValTyrAlaSerGlnGluThrPheGlyMetLysLeuSerVal
Protein: ADRP

| | |
|---|---|
| <210> | 7 |
| <211> | 437 |
| <212> | PRT |
| <213> | Homo sapiens |
| <221> | WIPO Standard ST.26 |
| <222> | 1...437 |
| <400> | 7 |

MASVAVDPQPSVVTRVVNLPLVSSTYDLMSSAYLSTKDQYPYLKSVCEMAE
NGVKTITSVAMTSALPIIQKLEPQIAVANTYACKGLDRIEERLPILNQPSTQIVANAKGAV
TGAKDAVTTTVTGAKDSVASTITGVMDKTKGAVTGSVEKTKSVVSGSINTVLGSRMMQ
LVSSGVENALTKSELLVEQYLPLTEEELEKEAKKVEGFDLVQKPSYYVRLGSLSTKLHSR
AYQQALSRVKEAKQKSQQTISQLHSTVHLIEFARKNVYSANQKIQDAQDKLYLSWVEW
KRSIGYDDTDESHCAEHIESRTLAIARNLTQQLQTTCHTLLSNIQGVPQNIQDQAKHMGV
MAGDIYSVFRNAASFKEVSDSLLTSSKGQLQKMKESLDDVMDYLVNNTPLNWLVGPFY
PQLTESQNAQDQGAEMDKSSQETQRSEHKTH
MetAlaSerValAlaValAspProGlnProSerValValThrArgValValAsnLeuProLeuValSer
SerThrTyrAspLeuMetSerSerAlaTyrLeuSerThrLysAspGlnTyrProTyrLeuLysSerValCysGluMet
AlaGluAsnGlyValLysThrIleThrSerValAlaMetThrSerAlaLeuProIleIleGlnLysLeuGluProGlnIleAla
ValAlaAsnThrTyrAlaCysLysGlyLeuAspArgIleGluGluArgLeuProIleLeuAsnGlnProSerThrGln
IleValAlaAsnAlaLysGlyAlaValThrGlyAlaLysAspAlaValThrThrThrValThrGlyAlaLysAspSerVal
AlaSerThrIleThrGlyValMetAspLysThrLysGlyAlaValThrGlySerValGluLysThrLysSerValValSer
GlySerIleAsnThrValLeuGlySerArgMetMetGlnLeuValSerSerGlyValGluAsnAlaLeuThrLysSer
GluLeuLeuValGluGlnTyrLeuProLeuThrGluGluGluLeuGluLysGluAlaLysLysValGluGlyPheAsp
LeuValGlnLysProSerTyrTyrValArgLeuGlySerLeuSerThrLysLeuHisSerArgAlaTyrGlnGlnAlaLeu
SerArgValLysGluAlaLysGlnLysSerGlnGlnThrIleSerGlnLeuHisSerThrValHisLeuIleGluPheAla
ArgLysAsnValTyrSerAlaAsnGlnLysIleGlnAspAlaGlnAspLysLeuTyrLeuSerTrpValGluTrpLys
ArgSerIleGlyTyrAspAspThrAspGluSerHisCysAlaGluHisIleGluSerArgThrLeuAlaIleAlaArgAsn
LeuThrGlnGlnLeuGlnThrThrCysHisThrLeuLeuSerAsnIleGlnGlyValProGlnAsnIleGlnAspGln
AlaLysHisMetGlyValMetAlaGlyAspIleTyrSerValPheArgAsnAlaAlaSerPheLysGluValSerAspSer
LeuLeuThrSerSerLysGlyGlnLeuGlnLysMetLysGluSerLeuAspAspValMetAspTyrLeuValAsnAs
nThrProLeuAsnTrpLeuValGlyProPheTyrProGlnLeuThrGluSerGlnAsnAlaGlnAspGlnGlyAla
GluMetAspLysSerSerGlnGluThrGlnArgSerGluHisLysThrHis
Protein: PI3K

| | |
|---|---|
| <210> | 8 |
| <211> | 1857 |
| <212> | PRT |
| <213> | Homo sapiens |
| <221> | WIPO Standard ST.26 |
| <222> | 1...1857 |
| <400> | 8 |

MKMSEGIISPLSLSSESSEQQQAAIRKFSNGSNGSGGGGGSNLSVNSSNSGSNN
SIRKSSTLMYNGPLPSINDGKELLLENSKPKVVELVNTFNHKPLSTIHSVHNEIPPPAIEKE
KKEIINTISNSGVTKYMTALEILDSTINTPLNRSRSGSIGSKPICNNLTSSSSSSSTTATTPSP
TTTSNNNNNNNNNNNNNNNNNNNNNNNNNNNNNNNNNNNNNNNNNNTTSTTTTTTS
ILISSSPPPSSSSSSSSNDEQFNNNNNNNNSNSGGSSRMITSKSQIKPLIVTSNTAATTTTTT
TTNTSAPTTPTNRVQSSLDDLLFNLPTIPSNVPTVNGGPKISAVPKKVSSSKLLIPPSSNVS
SSSNITLSLSSSSPSSSSSSSTSTVVPIVQLSSSNSTNSPSTSLPTTPRLSQPTTSYTQLIPSQQ
QQQPTESNSSSNTNTTTTSSSSSSSSSSLTISSPQPSNNSIRISAFGRSSTQFTISSNGIPSSPG
QVSNKDYNNIGNLSNSSGERVKNKQYSMLNISKKTILDESDISSSPRSIGSPNSIRASISSQ
LPPSLSSIGGGGGGGSGPNVVSNKPLVVKKPSTSEHIKKENIWRQTMIPLTKEDHIKVVFE
GIPGKRVIQKFLIDKTPIEIKSKFFEDLKDGDLLNGLTQLPSLIPEHYELKVLSVNSTISNET
LPLRRQTLMQACNISRLFPKLHLILKSESTTILDGASTTTTTTTTTTTTTANQSSNIITKSNS
SLDLTINNSNEIIDVKGHIQAELEIFELIGTSFTRVLDQGQEVVSFRRDFAQFRLSNFTSTR
NDLSQMIYVSSEPLPLTIPNKITIMVLLPGDGKIIKRVDCCPNSSVGDVKKEIFKKFAMIDR
VHTQGKTQDDFVLKVTGFREYILCIHELGNLTSRQRFYPTGSGGDFSLMDYDYIRQCVG
KNQTVELSLTNNSILSLNQVSEKVSFIDKILETSDFDDYDEDLDSINSNSFDDLKQSIQQQ
QQQQIQTVINIKETNKENKDSNKENKDSSSNNNNNNNNNNNNNNNNNNNNNNNNNNN
GNNNGNNSNNNSNSNISRGSIDSEGNGSGSGNGSEQPTLIGVQNFSLPNNSKLPINIVKRL
FRVNIAGLRNLNFNNNEDARNKFADGKNNQPNVFVMAELYYGGELLTNPVFTPIAQLA
SYGDGSVEFPNWEKGIAFTIPIRYLPRAARASFTVYVTTISEALESQMDEVVSKSIPIGWS
NCLLMNHKGMLRMGPTAFRLWDDGRRANPIGTCVDNQAAKQPIILLVEFESFIRPIVYV
DTALQSMMVNDSSSISSNGVESPSIVSFSSSAASSSPLPSSPLPSPVGLKKLDLDEARRLKA
LMDSDPLVQLSAEDKKLVYGYRHIYKSKPKALAKFLLSVNWIDPDQVTDAYRQMNDW
ALLKPVQALEILDAKFADEHVRNFAIKIINSFSDAEFSDFLLQLTQVLKYEPYHNSDLTHI
LIQRALSNRSRIGHFFFWFLKSEMHTPEIEERYGLLLEGYLRSCGTHRQDLIKQNQVLKSL
HTVAMAVKQTNGSSERKKVLMEGLSKIKFPDTFQLPLDPRWEAKGLIIDKCRYMDSKK
LPLWLVFENVEPHAKPLTVIFKVGDDLRQDILTLQVLRIMDKFWKNSGMDLRLQPYKCI
ATGDGIGMLEVVLNANTIANINKDAGGTGALLEEKTLVNWLKECNKTEAEYNKAVETF
ILSCAGYVVATYVMGIGDRHSDNIMITKLGHLFHIDFGHFLGNYKKKYGFKRERAPFIFT
PQYMAIVGGKDSENFKRFVTTCCSAYNILRKNTDLFINLFQLMLSTGIPELQVAEDIDYL
RKALAPGLSDEEAAEEFTKNISVALNTKTVLLNDIFHGWAH
MetLysMetSerGluGlyIleIleSerProLeuSerLeuSerSerGluSerSerGluGlnGlnGlnAlaAla
IleArgLysPheSerAsnGlySerAsnGlySerGlyGlyGlyGlyGlySerAsnLeuSerValAsnSerSerAsnSerGly
SerAsnAsnSerIleArgLysSerSerThrLeuMetTyrAsnGlyProLeuProSerIleAsnAspGlyLysGluLeu
LeuLeuGluAsnSerLysProLysValValGluLeuValAsnThrPheAsnHisLysProLeuSerThrIleHisSerVal
HisAsnGluIleProProProAlaIleGluLysGluLysLysGluIleIleAsnThrIleSerAsnSerGlyValThrLysTyr
MetThrAlaLeuGluIleLeuAspSerThrIleAsnThrProLeuAsnArgSerArgSerGlySerIleGlySerLysPro
IleCysAsnAsnLeuThrSerSerSerSerSerSerSerThrThrAlaThrThrProSerProThrThrThrSerAsnAsn
AsnAsnAsnAsnAsnAsnAsnAsnAsnAsnAsnAsnAsnAsnAsnAsnAsnAsnAsnAsnAsnAsnAsnAsn
AsnAsnAsnAsnAsnAsnAsnAsnAsnAsnAsnAsnAsnAsnAsnAsnThrThrSerThrThrThrThrThrThr
SerIleLeuIleSerSerSerProProProSerSerSerSerSerSerSerSerAsnAspGluGlnPheAsnAsnAsnAsnAsn
AsnAsnAsnSerAsnSerGlyGlySerSerArgMetIleThrSerLysSerGlnIleLysProLeuIleValThrSerAsn
ThrAlaAlaThrThrThrThrThrThrThrThrAsnThrSerAlaProThrThrProThrAsnArgValGlnSerSer
LeuAspAspLeuLeuPheAsnLeuProThrIleProSerAsnValProThrValAsnGlyGlyProLysIleSerAlaVal
ProLysLysValSerSerSerLysLeuLeuIleProProSerSerAsnValSerSerSerSerAsnIleThrLeuSerLeuSer
SerSerSerProSerSerSerSerSerSerSerThrSerThrValValProIleValGlnLeuSerSerSerAsnSerThrAsn
SerProSerThrSerLeuProThrThrProArgLeuSerGlnProThrThrSerTyrThrGlnLeuIleProSerGlnGln
GlnGlnGlnProThrGluSerAsnSerSerSerAsnThrAsnThrThrThrThrSerSerSerSerSerSerSerSerSer
SerLeuThrIleSerSerProGlnProSerAsnAsnSerIleArgIleSerAlaPheGlyArgSerSerThrGlnPheThrIle
SerSerAsnGlyIleProSerSerProGlyGlnValSerAsnLysAspTyrAsnAsnIleGlyAsnLeuSerAsnSerSer
GlyGluArgValLysAsnLysGlnTyrSerMetLeuAsnIleSerLysLysThrIleLeuAspGluSerAspIleSerSer
SerProArgSerIleGlySerProAsnSerIleArgAlaSerIleSerSerGlnLeuProProSerLeuSerSerIleGlyGly
GlyGlyGlyGlyGlySerGlyProAsnValValSerAsnLysProLeuValValLysLysProSerThrSerGluHisIle
LysLysGluAsnIleTrpArgGlnThrMetIleProLeuThrLysGluAspHisIleLysValValPheGluGlyIlePro
GlyLysArgValIleGlnLysPheLeuIleAspLysThrProIleGluIleLysSerLysPhePheGluAspLeuLysAsp
GlyAspLeuLeuAsnGlyLeuThrGlnLeuProSerLeuIleProGluHisTyrGluLeuLysValLeuSerValAsnSer
ThrIleSerAsnGluThrLeuProLeuArgArgGlnThrLeuMetGlnAlaCysAsnIleSerArgLeuPheProLys
LeuHisLeuIleLeuLysSerGluSerThrThrIleLeuAspGlyAlaSerThrThrThrThrThrThrThrThrThrThr
ThrThrThrAlaAsnGlnSerSerAsnIleIleThrLysSerAsnSerSerLeuAspLeuThrIleAsnAsnSerAsnGlu
IleIleAspValLysGlyHisIleGlnAlaGluLeuGluIlePheGluLeuIleGlyThrSerPheThrArgValLeuAsp
GlnGlyGlnGluValValSerPheArgArgAspPheAlaGlnPheArgLeuSerAsnPheThrSerThrArgAsnAsp
LeuSerGlnMetIleTyrValSerSerGluProLeuProLeuThrIleProAsnLysIleThrIleMetValLeuLeuProGly
AspGlyLysIleIleLysArgValAspCysCysProAsnSerSerValGlyAspValLysLysGluIlePheLysLys
PheAlaMetIleAspArgValHisThrGlnGlyLysThrGlnAspAspPheValLeuLysValThrGlyPheArgGlu
TyrIleLeuCysIleHisGluLeuGlyAsnLeuThrSerArgGlnArgPheTyrProThrGlySerGlyGlyAspPheSer
LeuMetAspTyrAspTyrIleArgGlnCysValGlyLysAsnGlnThrValGluLeuSerLeuThrAsnAsnSerIle
LeuSerLeuAsnGlnValSerGluLysValSerPheIleAspLysIleLeuGluThrSerAspPheAspAspTyrAspGlu
AspLeuAspSerIleAsnSerAsnSerPheAspAspLeuLysGlnSerIleGlnGlnGinGinGinGlnGlnIleGln
ThrValIleAsnIleLysGluThrAsnLysGluAsnLysAspSerAsnLysGluAsnLysAspSerSerSerAsnAsnAsn
AsnAsnAsnAsnAsnAsnAsnAsnAsnAsnAsnAsnAsnAsnAsnAsnAsnAsnAsnAsnAsnAsnAsnAsn
GlyAsnAsnAsnGlyAsnAsnSerAsnAsnAsnSerAsnSerAsnIleSerArgGlySerIleAspSerGluGlyAsn
GlySerGlySerGlyAsnGlySerGluGlnProThrLeuIleGlyValGlnAsnPheSerLeuProAsnAsnSerLys
LeuProIleAsnIleValLysArgLeuPheArgValAsnIleAlaGlyLeuArgAsnLeuAsnPheAsnAsnAsnGluAs
pAlaArgAsnLysPheAlaAspGlyLysAsnAsnGlnProAsnValPheValMetAlaGluLeuTyrTyrGlyGly
GluLeuLeuThrAsnProValPheThrProIleAlaGlnLeuAlaSerTyrGlyAspGlySerValGluPheProAsnTrp
GluLysGlyIleAlaPheThrIleProIleArgTyrLeuProArgAlaAlaArgAlaSerPheThrValTyrValThrThr
IleSerGluAlaLeuGluSerGlnMetAspGluValValSerLysSerIleProIleGlyTrpSerAsnCysLeuLeuMet
AsnHisLysGlyMetLeuArgMetGlyProThrAlaPheArgLeuTrpAspAspGlyArgArgAlaAsnProIleGly
ThrCysValAspAsnGlnAlaAlaLysGlnProIleIleLeuLeuValGluPheGluSerPheIleArgProIleValTyr
ValAspThrAlaLeuGlnSerMetMetValAsnAspSerSerSerIleSerSerAsnGlyValGluSerProSerIleValSer
PheSerSerSerAlaAlaSerSerSerProLeuProSerSerProLeuProSerProValGlyLeuLysLysLeuAspLeu
AspGluAlaArgArgLeuLysAlaLeuMetAspSerAspProLeuValGlnLeuSerAlaGluAspLysLysLeu
ValTyrGlyTyrArgHisIleTyrLysSerLysProLysAlaLeuAlaLysPheLeuLeuSerValAsnTrpIleAspPro
AspGlnValThrAspAlaTyrArgGlnMetAsnAspTrpAlaLeuLeuLysProValGlnAlaLeuGluIleLeuAsp
AlaLysPheAlaAspGluHisValArgAsnPheAlaIleLysIleIleAsnSerPheSerAspAlaGluPheSerAspPhe
LeuLeuGlnLeuThrGlnValLeuLysTyrGluProTyrHisAsnSerAspLeuThrHisIleLeuIleGlnArgAlaLeu
SerAsnArgSerArgIleGlyHisPhePhePheTrpPheLeuLysSerGluMetHisThrProGluIleGluGluArgTyr
GlyLeuLeuLeuGluGlyTyrLeuArgSerCysGlyThrHisArgGlnAspLeuIleLysGlnAsnGlnValLeuLys
SerLeuHisThrValAlaMetAlaValLysGlnThrAsnGlySerSerGluArgLysLysValLeuMetGluGlyLeu
SerLysIleLysPheProAspThrPheGlnLeuProLeuAspProArgTrpGluAlaLysGlyLeuIleIleAspLys
CysArgTyrMetAspSerLysLysLeuProLeuTrpLeuValPheGluAsnValGluProHisAlaLysProLeuThrVal
IlePheLysValGlyAspAspLeuArgGlnAspIleLeuThrLeuGlnValLeuArgIleMetAspLysPheTrpLys
AsnSerGlyMetAspLeuArgLeuGlnProTyrLysCysIleAlaThrGlyAspGlyIleGlyMetLeuGluValVal
LeuAsnAlaAsnThrIleAlaAsnIleAsnLysAspAlaGlyGlyThrGlyAlaLeuLeuGluGluLysThrLeuVal
AsnTrpLeuLysGluCysAsnLysThrGluAlaGluTyrAsnLysAlaValGluThrPheIleLeuSerCysAlaGlyTyr
ValValAlaThrTyrValMetGlyIleGlyAspArgHisSerAspAsnIleMetIleThrLysLeuGlyHisLeuPheHis
IleAspPheGlyHisPheLeuGlyAsnTyrLysLysLysTyrGlyPheLysArgGluArgAlaProPheIlePheThr
ProGlnTyrMetAlaIleValGlyGlyLysAspSerGluAsnPheLysArgPheValThrThrCysCysSerAlaTyrAsn
IleLeuArgLysAsnThrAspLeuPheIleAsnLeuPheGlnLeuMetLeuSerThrGlyIleProGluLeuGlnVal
AlaGluAspIleAspTyrLeuArgLysAlaLeuAlaProGlyLeuSerAspGluGluAlaAlaGluGluPheThrLys
AsnIleSerValAlaLeuAsnThrLysThrValLeuLeuAsnAspIlePheHisGlyTrpAlaHis
Protein: ATG7

| | |
|---|---|
| <210> | 9 |
| <211> | 703 |
| <212> | PRT |
| <213> | Homo sapiens |
| <221> | WIPO Standard ST.26 |
| <222> | 1...703 |
| <400> | 9 |

MAAATGDPGLSKLQFAPFSSALDVGFWHELTQKKLNEYRLDEAPKDIKGYY
YNGDSAGLPARLTLEFSAFDMSAPTPARCCPAIGTLYNTNTLESFKTADKKLLLEQAAN
EIWESIKSGTALENPVLLNKFLLLTFADLKKYHFYYWFCYPALCLPESLPLIQGPVGLDQ
RFSLKQIEALECAYDNLCQTEGVTALPYFLIKYDENMVLVSLLKHYSDFFQGQRTKITIG
VYDPCNLAQYPGWPLRNFLVLAAHRWSSSFQSVEVVCFRDRTMQGARDVAHSIIFEVK
LPEMAFSPDCPKAVGWEKNQKGGMGPRMVNLSECMDPKRLAESSVDLNLKLMCWRL
VPTLDLDKVVSVKCLLLGAGTLGCNVARTLMGWGVRHITFVDNAKISYSNPVRQPLYE
FEDCLGGGKPKALAAADRLQKIFPGVNARGFNMSIPMPGHPVNFSSVTLEQARRDVEQL
EQLIESHDVVFLLMDTRESRWLPAVIAASKRKLVINAALGFDTFVVMRHGLKKPKQQG
AGDLCPNHPVASADLLGSSLFANIPGYKLGCYFCNDVVAPGDSTRDRTLDQQCTVSRPG
LAVIAGALAVELMVSVLQHPEGGYAIASSSDDRMNEPPTSLGLVPHQIRGFLSRFDNVLP
VSLAFDKCTACSSKVLDQYEREGFNFLAKVFNSSHSFLEDLTGLTLLHQETQAAEIWDM
SDDETI
MetAlaAlaAlaThrGlyAspProGlyLeuSerLysLeuGlnPheAlaProPheSerSerAlaLeuAsp
ValGlyPheTrpHisGluLeuThrGlnLysLysLeuAsnGluTyrArgLeuAspGluAlaProLysAspIleLysGly
TyrTyrTyrAsnGlyAspSerAlaGlyLeuProAlaArgLeuThrLeuGluPheSerAlaPheAspMetSerAlaPro
ThrProAlaArgCysCysProAlaIleGlyThrLeuTyrAsnThrAsnThrLeuGluSerPheLysThrAlaAspLys
LysLeuLeuLeuGluGlnAlaAlaAsnGluIleTrpGluSerIleLysSerGlyThrAlaLeuGluAsnProValLeuLeu
AsnLysPheLeuLeuLeuThrPheAlaAspLeuLysLysTyrHisPheTyrTyrTrpPheCysTyrProAlaLeuCys
LeuProGluSerLeuProLeuIleGlnGlyProValGlyLeuAspGlnArgPheSerLeuLysGlnIleGluAlaLeu
GluCysAlaTyrAspAsnLeuCysGlnThrGluGlyValThrAlaLeuProTyrPheLeuIleLysTyrAspGluAsn
MetValLeuValSerLeuLeuLysHisTyrSerAspPhePheGlnGlyGlnArgThrLysIleThrIleGlyValTyrAsp
ProCysAsnLeuAlaGlnTyrProGlyTrpProLeuArgAsnPheLeuValLeuAlaAlaHisArgTrpSerSerSer
PheGlnSerValGluValValCysPheArgAspArgThrMetGlnGlyAlaArgAspValAlaHisSerIleIlePheGlu
ValLysLeuProGluMetAlaPheSerProAspCysProLysAlaValGlyTrpGluLysAsnGlnLysGlyGlyMet
GlyProArgMetValAsnLeuSerGluCysMetAspProLysArgLeuAlaGluSerSerValAspLeuAsnLeuLys
LeuMetCysTrpArgLeuValProThrLeuAspLeuAspLysValValSerValLysCysLeuLeuLeuGlyAla
GlyThrLeuGlyCysAsnValAlaArgThrLeuMetGlyTrpGlyValArgHisIleThrPheValAspAsnAlaLysIle
SerTyrSerAsnProValArgGlnProLeuTyrGluPheGluAspCysLeuGlyGlyGlyLysProLysAlaLeuAla
AlaAlaAspArgLeuGlnLysIlePheProGlyValAsnAlaArgGlyPheAsnMetSerIleProMetProGlyHis
ProValAsnPheSerSerValThrLeuGluGlnAlaArgArgAspValGluGlnLeuGluGlnLeuIleGluSerHisAsp
ValValPheLeuLeuMetAspThrArgGluSerArgTrpLeuProAlaValIleAlaAlaSerLysArgLysLeuVal
IleAsnAlaAlaLeuGlyPheAspThrPheValValMetArgHisGlyLeuLysLysProLysGlnGlnGlyAlaGly
AspLeuCysProAsnHisProValAlaSerAlaAspLeuLeuGlySerSerLeuPheAlaAsnIleProGlyTyrLysLeu
GlyCysTyrPheCysAsnAspValValAlaProGlyAspSerThrArgAspArgThrLeuAspGlnGlnCysThr
ValSerArgProGlyLeuAlaValIleAlaGlyAlaLeuAlaValGluLeuMetValSerValLeuGlnHisProGluGly
GlyTyrAlaIleAlaSerSerSerAspAspArgMetAsnGluProProThrSerLeuGlyLeuValProHisGlnIleArg
GlyPheLeuSerArgPheAspAsnValLeuProValSerLeuAlaPheAspLysCysThrAlaCysSerSerLysVal
LeuAspGlnTyrGluArgGluGlyPheAsnPheLeuAlaLysValPheAsnSerSerHisSerPheLeuGluAspLeu
ThrGlyLeuThrLeuLeuHisGlnGluThrGlnAlaAlaGluIleTrpAspMetSerAspAspGluThrIle
Protein: Pcsk9

| | |
|---|---|
| <210> | |
| <211> | 692 |
| <212> | PRT |
| <213> | Homo sapiens |
| <221> | WIPO Standard ST.26 |
| <222> | 1...692 |
| <400> | 10 |

MGTVSSRRSWWPLPLLLLLLLLLGPAGARAQEDEDGDYEELVLALRSEEDGL
AEAPEHGTTATFHRCAKDPWRLPGTYVVVLKEETHLSQSERTARRLQAQAARRGYLTK
ILHVFHGLLPGFLVKMSGDLLELALKLPHVDYIEEDSSVFAQSIPWNLERITPPRYRADEY
QPPDGGSLVEVYLLDTSIQSDHREIEGRVMVTDFENVPEEDGTRFHRQASKCDSHGTHL
AGVVSGRDAGVAKGASMRSLRVLNCQGKGTVSGTLIGLEFIRKSQLVQPVGPLVVLLPL
AGGYSRVLNAACQRLARAGVVLVTAAGNFRDDACLYSPASAPEVITVGATNAQDQPV
TLGTLGTNFGRCVDLFAPGEDIIGASSDCSTCFVSQSGTSQAAAHVAGIAAMMLSAEPEL
TLAELRQRLIHFSAKDVINEAWFPEDQRVLTPNLVAALPPSTHGAGWQLFCRTVWSAHS
GPTRMATAVARCAPDEELLSCSSFSRSGKRRGERMEAQGGKLVCRAHNAFGGEGVYAI
ARCCLLPQANCSVHTAPPAEASMGTRVHCHQQGHVLTGCSSHWEVEDLGTHKPPVLRP
RGQPNQCVGHREASIHASCCHAPGLECKVKEHGIPAPQEQVTVACEEGWTLTGCSALPG
TSHVLGAYAVDNTCVVRSRDVSTTGSTSEGAVTAVAICCRSRHLAQASQELQ
MetGlyThrValSerSerArgArgSerTrpTrpProLeuProLeuLeuLeuLeuLeuLeuLeuLeuLeu
GlyProAlaGlyAlaArgAlaGlnGluAspGluAspGlyAspTyrGluGluLeuValLeuAlaLeuArgSerGluGlu
AspGlyLeuAlaGluAlaProGluHisGlyThrThrAlaThrPheHisArgCysAlaLysAspProTrpArgLeuPro
GlyThrTyrValValValLeuLysGluGluThrHisLeuSerGlnSerGluArgThrAlaArgArgLeuGlnAlaGln
AlaAlaArgArgGlyTyrLeuThrLysIleLeuHisValPheHisGlyLeuLeuProGlyPheLeuValLysMetSer
GlyAspLeuLeuGluLeuAlaLeuLysLeuProHisValAspTyrIleGluGluAspSerSerValPheAlaGlnSerIle
ProTrpAsnLeuGluArgIleThrProProArgTyrArgAlaAspGluTyrGlnProProAspGlyGlySerLeuVal
GluValTyrLeuLeuAspThrSerIleGlnSerAspHisArgGluIleGluGlyArgValMetValThrAspPheGluAsn
ValProGluGluAspGlyThrArgPheHisArgGlnAlaSerLysCysAspSerHisGlyThrHisLeuAlaGlyVal
ValSerGlyArgAspAlaGlyValAlaLysGlyAlaSerMetArgSerLeuArgValLeuAsnCysGlnGlyLysGly
ThrValSerGlyThrLeuIleGlyLeuGluPheIleArgLysSerGlnLeuValGlnProValGlyProLeuValValLeu
LeuProLeuAlaGlyGlyTyrSerArgValLeuAsnAlaAlaCysGlnArgLeuAlaArgAlaGlyValValLeuVal
ThrAlaAlaGlyAsnPheArgAspAspAlaCysLeuTyrSerProAlaSerAlaProGluValIleThrValGlyAla
ThrAsnAlaGlnAspGlnProValThrLeuGlyThrLeuGlyThrAsnPheGlyArgCysValAspLeuPheAlaPro
GlyGluAspIleIleGlyAlaSerSerAspCysSerThrCysPheValSerGlnSerGlyThrSerGlnAlaAlaAlaHisVal
AlaGlyIleAlaAlaMetMetLeuSerAlaGluProGluLeuThrLeuAlaGluLeuArgGlnArgLeuIleHisPhe
SerAlaLysAspValIleAsnGluAlaTrpPheProGluAspGlnArgValLeuThrProAsnLeuValAlaAlaLeuPro
ProSerThrHisGlyAlaGlyTrpGlnLeuPheCysArgThrValTrpSerAlaHisSerGlyProThrArgMetAla
ThrAlaValAlaArgCysAlaProAspGluGluLeuLeuSerCysSerSerPheSerArgSerGlyLysArgArgGlyGlu
ArgMetGluAlaGlnGlyGlyLysLeuValCysArgAlaHisAsnAlaPheGlyGlyGluGlyValTyrAlaIleAla
ArgCysCysLeuLeuProGlnAlaAsnCysSerValHisThrAlaProProAlaGluAlaSerMetGlyThrArgVal
HisCysHisGlnGlnGlyHisValLeuThrGlyCysSerSerHisTrpGluValGluAspLeuGlyThrHisLysPro
ProValLeuArgProArgGlyGlnProAsnGlnCysValGlyHisArgGluAlaSerIleHisAlaSerCysCysHisAla
ProGlyLeuGluCysLysValLysGluHisGlyIleProAlaProGlnGluGlnValThrValAlaCysGluGluGlyTrp
ThrLeuThrGlyCysSerAlaLeuProGlyThrSerHisValLeuGlyAlaTyrAlaValAspAsnThrCysValVal
ArgSerArgAspValSerThrThrGlySerThrSerGluGlyAlaValThrAlaValAlaIleCysCysArgSerArgHis
LeuAlaGlnAlaSerGlnGluLeuGln
Protein: Scap

| | |
|---|---|
| <210> | 11 |
| <211> | 1279 |
| <212> | PRT |
| <213> | Homo sapiens |
| <221> | WIPO Standard ST.26 |
| <222> | 1...1279 |
| <400> | 11 |

MTLTERLREKISRAFYNHGLLCASYPIPIILFTGFCILACCYPLLKLPLPGTGPV
EFTTPVKDYSPPPVDSDRKQGEPTEQPEWYVGAPVAYVQQIFVKSSVFPWHKNLLAVD
VFRSPLSRAFQLVEEIRNHVLRDSSGIRSLEELCLQVTDLLPGLRKLRNLLPEHGCLLLSP
GNFWQNDWERFHADPDIIGTIHQHEPKTLQTSATLKDLLFGVPGKYSGVSLYTRKRMVS
YTITLVFQHYHAKFLGSLRARLMLLHPSPNCSLRAESLVHVHFKEEIGVAELIPLVTTYIIL
FAYIYFSTRKIDMVKSKWGLALAAVVTVLSSLLMSVGLCTLFGLTPTLNGGEIFPYLVV
VIGLENVLVLTKSVVSTPVDLEVKLRIAQGLSSESWSIMKNMATELGIILIGYFTLVPAIQ
EFCLFAVVGLVSDFFLQMLFFTTVLSIDIRRMELADLNKRLPPEACLPSAKPVGQPTRYE
RQLAVRPSTPHTITLQPSSFRNLRLPKRLRVVYFLARTRLAQRLIMAGTVVWIGILVYTD
PAGLRNYLAAQVTEQSPLGEGALAPMPVPSGMLPPSHPDPAFSIFPPDAPKLPENQTSPG
ESPERGGPAEVVHDSPVPEVTWGPEDEELWRKLSFRHWPTLFSYYNITLAKRYISLLPVI
PVTLRLNPREALEGRHPQDGRSAWPPPGPIPAGHWEAGPKGPGGVQAHGDVTLYKVAA
LGLATGIVLVLLLLCLYRVLCPRNYGQLGGGPGRRRRGELPCDDYGYAPPETEIVPLVLR
GHLMDIECLASDGMLLVSCCLAGHVCVWDAQTGDCLTRIPRPGRQRRDSGVGSGLEAQ
ESWERLSDGGKAGPEEPGDSPPLRHRPRGPPPPSLFGDQPDLTCLIDTNFSAQPRSSQPTQ
PEPRHRAVCGRSRDSPGYDFSCLVQRVYQEEGLAAVCTPALRPPSPGPVLSQAPEDEGG
SPEKGSPSLAWAPSAEGSIWSLELQGNLIVVGRSSGRLEVWDAIEGVLCCSSEEVSSGITA
LVFLDKRIVAARLNGSLDFFSLETHTALSPLQFRGTPGRGSSPASPVYSSSDTVACHLTHT
VPCAHQKPITALKAAAGRLVTGSQDHTLRVFRLEDSCCLFTLQGHSGAITTVYIDQTMV
LASGGQDGAICLWDVLTGSRVSHVFAHRGDVTSLTCTTSCVISSGLDDLISIWDRSTGIK
FYSIQQDLGCGASLGVISDNLLVTGGQGCVSFWDLNYGDLLQTVYLGKNSEAQPARQIL
VLDNAAIVCNFGSELSLVYVPSVLEKLD
MetThrLeuThrGluArgLeuArgGluLysIleSerArgAlaPheTyrAsnHisGlyLeuLeuCysAla
SerTyrProIleProIleIleLeuPheThrGlyPheCysIleLeuAlaCysCysTyrProLeuLeuLysLeuProLeuPro
GlyThrGlyProValGluPheThrThrProValLysAspTyrSerProProProValAspSerAspArgLysGlnGlyGlu
ProThrGluGlnProGluTrpTyrValGlyAlaProValAlaTyrValGlnGlnIlePheValLysSerSerValPhePro
TrpHisLysAsnLeuLeuAlaValAspValPheArgSerProLeuSerArgAlaPheGlnLeuValGluGluIleArg
AsnHisValLeuArgAspSerSerGlyIleArgSerLeuGluGluLeuCysLeuGlnValThrAspLeuLeuProGly
LeuArgLysLeuArgAsnLeuLeuProGluHisGlyCysLeuLeuLeuSerProGlyAsnPheTrpGlnAsnAspTrp
GluArgPheHisAlaAspProAspIleIleGlyThrIleHisGlnHisGluProLysThrLeuGlnThrSerAlaThrLeu
LysAspLeuLeuPheGlyValProGlyLysTyrSerGlyValSerLeuTyrThrArgLysArgMetValSerTyrThr
IleThrLeuValPheGlnHisTyrHisAlaLysPheLeuGlySerLeuArgAlaArgLeuMetLeuLeuHisProSer
ProAsnCysSerLeuArgAlaGluSerLeuValHisValHisPheLysGluGluIleGlyValAlaGluLeuIleProLeu
ValThrThrTyrIleIleLeuPheAlaTyrIleTyrPheSerThrArgLysIleAspMetValLysSerLysTrpGlyLeuAla
LeuAlaAlaValValThrValLeuSerSerLeuLeuMetSerValGlyLeuCysThrLeuPheGlyLeuThrProThr
LeuAsnGlyGlyGluIlePheProTyrLeuValValValIleGlyLeuGluAsnValLeuValLeuThrLysSerValVal
SerThrProValAspLeuGluValLysLeuArgIleAlaGlnGlyLeuSerSerGluSerTrpSerIleMetLysAsn
MetAlaThrGluLeuGlyIleIleLeuIleGlyTyrPheThrLeuValProAlaIleGlnGluPheCysLeuPheAlaValVal
GlyLeuValSerAspPhePheLeuGlnMetLeuPhePheThrThrValLeuSerIleAspIleArgArgMetGluLeu
AlaAspLeuAsnLysArgLeuProProGluAlaCysLeuProSerAlaLysProValGlyGlnProThrArgTyrGlu
ArgGlnLeuAlaValArgProSerThrProHisThrIleThrLeuGlnProSerSerPheArgAsnLeuArgLeuPro
LysArgLeuArgValValTyrPheLeuAlaArgThrArgLeuAlaGlnArgLeuIleMetAlaGlyThrValValTrpIle
GlyIleLeuValTyrThrAspProAlaGlyLeuArgAsnTyrLeuAlaAlaGlnValThrGluGlnSerProLeuGly
GluGlyAlaLeuAlaProMetProValProSerGlyMetLeuProProSerHisProAspProAlaPheSerIlePheProPr
oAspAlaProLysLeuProGluAsnGlnThrSerProGlyGluSerProGluArgGlyGlyProAlaGluValValHis
AspSerProValProGluValThrTrpGlyProGluAspGluGluLeuTrpArgLysLeuSerPheArgHisTrpPro
ThrLeuPheSerTyrTyrAsnIleThrLeuAlaLysArgTyrIleSerLeuLeuProValIleProValThrLeuArgLeu
AsnProArgGluAlaLeuGluGlyArgHisProGlnAspGlyArgSerAlaTrpProProProGlyProIleProAlaGly
HisTrpGluAlaGlyProLysGlyProGlyGlyValGlnAlaHisGlyAspValThrLeuTyrLysValAlaAlaLeu
GlyLeuAlaThrGlyIleValLeuValLeuLeuLeuLeuCysLeuTyrArgValLeuCysProArgAsnTyrGlyGln
LeuGlyGlyGlyProGlyArgArgArgArgGlyGluLeuProCysAspAspTyrGlyTyrAlaProProGluThrGlu
IleValProLeuValLeuArgGlyHisLeuMetAspIleGluCysLeuAlaSerAspGlyMetLeuLeuValSerCys
CysLeuAlaGlyHisValCysValTrpAspAlaGlnThrGlyAspCysLeuThrArgIleProArgProGlyArgGlnArg
ArgAspSerGlyValGlySerGlyLeuGluAlaGlnGluSerTrpGluArgLeuSerAspGlyGlyLysAlaGlyPro
GluGluProGlyAspSerProProLeuArgHisArgProArgGlyProProProProSerLeuPheGlyAspGlnPro
AspLeuThrCysLeuIleAspThrAsnPheSerAlaGlnProArgSerSerGlnProThrGlnProGluProArgHisArg
AlaValCysGlyArgSerArgAspSerProGlyTyrAspPheSerCysLeuValGlnArgValTyrGlnGluGluGly
LeuAlaAlaValCysThrProAlaLeuArgProProSerProGlyProValLeuSerGlnAlaProGluAspGluGly
GlySerProGluLysGlySerProSerLeuAlaTrpAlaProSerAlaGluGlySerIleTrpSerLeuGluLeuGlnGlyAs
nLeuIleValValGlyArgSerSerGlyArgLeuGluValTrpAspAlaIleGluGlyValLeuCysCysSerSerGlu
GluValSerSerGlyIleThrAlaLeuValPheLeuAspLysArgIleValAlaAlaArgLeuAsnGlySerLeuAspPhe
PheSerLeuGluThrHisThrAlaLeuSerProLeuGlnPheArgGlyThrProGlyArgGlySerSerProAlaSer
ProValTyrSerSerSerAspThrValAlaCysHisLeuThrHisThrValProCysAlaHisGlnLysProIleThrAla
LeuLysAlaAlaAlaGlyArgLeuValThrGlySerGlnAspHisThrLeuArgValPheArgLeuGluAspSerCysCy
sLeuPheThrLeuGlnGlyHisSerGlyAlaIleThrThrValTyrIleAspGlnThrMetValLeuAlaSerGlyGly
GlnAspGlyAlaIleCysLeuTrpAspValLeuThrGlySerArgValSerHisValPheAlaHisArgGlyAspValThr
SerLeuThrCysThrThrSerCysValIleSerSerGlyLeuAspAspLeuIleSerIleTrpAspArgSerThrGlyIle
LysPheTyrSerIleGlnGlnAspLeuGlyCysGlyAlaSerLeuGlyValIleSerAspAsnLeuLeuValThrGlyGly
GlnGlyCysValSerPheTrpAspLeuAsnTyrGlyAspLeuLeuGlnThrValTyrLeuGlyLysAsnSerGluAla
GlnProAlaArgGlnIleLeuValLeuAspAsnAlaAlaIleValCysAsnPheGlySerGluLeuSerLeuValTyr
ValProSerValLeuGluLysLeuAsp
Protein: Irp10

| | |
|---|---|
| <210> | 12 |
| <211> | 713 |
| <212> | PRT |
| <213> | Homo sapiens |
| <221> | WIPO Standard ST.26 |
| <222> | 1...713 |
| <400> | 12 |

MLLATLLLLLLGGALAHPDRIIFPNHACEDPPAVLLEVQGTLQRPLVRDSRTS
PANCTWLILGSKEQTVTIRFQKLHLACGSERLTLRSPLQPLISLCEAPPSPLQLPGGNVTIT
YSYAGARAPMGQGFLLSYSQDWLMCLQEEFQCLNHRCVSAVQRCDGVDACGDGSDE
AGCSSDPFPGLTPRPVPSLPCNVTLEDFYGVFSSPGYTHLASVSHPQSCHWLLDPHDGRR
LAVRFTALDLGFGDAVHVYDGPGPPESSRLLRSLTHFSNGKAVTVETLSGQAVVSYHTV
AWSNGRGFNATYHVRGYCLPWDRPCGLGSGLGAGEGLGERCYSEAQRCDGSWDCAD
GTDEEDCPGCPPGHFPCGAAGTSGATACYLPADRCNYQTFCADGADERRCRHCQPGNF
RCRDEKCVYETWVCDGQPDCADGSDEWDCSYVLPRKVITAAVIGSLVCGLLLVIALGC
TCKLYAIRTQEYSIFAPLSRMEAEIVQQQAPPSYGQLIAQGAIPPVEDFPTENPNDNSVLG
NLRSLLQILRQDMTPGGGPGARRRQRGRLMRRLVRRLRRWGLLPRINTPARASEARSQ
VTPSAAPLEALDGGTGPAREGGAVGGQDGEQAPPLPIKAPLPSASTSPAPTTVPEAPGPL
PSLPLEPSLLSGVVQALRGRLLPSLGPPGPTRSPPGPHTAVLALEDEDDVLLVPLAEPGV
WVAEAEDEPLLT
MetLeuLeuAlaThrLeuLeuLeuLeuLeuLeuGlyGlyAlaLeuAlaHisProAspArgIleIlePhe
ProAsnHisAlaCysGluAspProProAlaValLeuLeuGluValGlnGlyThrLeuGlnArgProLeuValArgAsp
SerArgThrSerProAlaAsnCysThrTrpLeuIleLeuGlySerLysGluGlnThrValThrIleArgPheGlnLysLeu
HisLeuAlaCysGlySerGluArgLeuThrLeuArgSerProLeuGlnProLeuIleSerLeuCysGluAlaProPro
SerProLeuGlnLeuProGlyGlyAsnValThrIleThrTyrSerTyrAlaGlyAlaArgAlaProMetGlyGlnGlyPhe
LeuLeuSerTyrSerGlnAspTrpLeuMetCysLeuGlnGluGluPheGlnCysLeuAsnHisArgCysValSerAla
ValGlnArgCysAspGlyValAspAlaCysGlyAspGlySerAspGluAlaGlyCysSerSerAspProPheProGly
LeuThrProArgProValProSerLeuProCysAsnValThrLeuGluAspPheTyrGlyValPheSerSerProGly
TyrThrHisLeuAlaSerValSerHisProGlnSerCysHisTrpLeuLeuAspProHisAspGlyArgArgLeuAlaVal
ArgPheThrAlaLeuAspLeuGlyPheGlyAspAlaValHisValTyrAspGlyProGlyProProGluSerSerArg
LeuLeuArgSerLeuThrHisPheSerAsnGlyLysAlaValThrValGluThrLeuSerGlyGlnAlaValValSer
TyrHisThrValAlaTrpSerAsnGlyArgGlyPheAsnAlaThrTyrHisValArgGlyTyrCysLeuProTrpAsp
ArgProCysGlyLeuGlySerGlyLeuGlyAlaGlyGluGlyLeuGlyGluArgCysTyrSerGluAlaGlnArgCys
AspGlySerTrpAspCysAlaAspGlyThrAspGluGluAspCysProGlyCysProProGlyHisPheProCysGly
AlaAlaGlyThrSerGlyAlaThrAlaCysTyrLeuProAlaAspArgCysAsnTyrGlnThrPheCysAlaAspGly
AlaAspGluArgArgCysArgHisCysGlnProGlyAsnPheArgCysArgAspGluLysCysValTyrGluThrTrp
ValCysAspGlyGlnProAspCysAlaAspGlySerAspGluTrpAspCysSerTyrValLeuProArgLysValIle
ThrAlaAlaValIleGlySerLeuValCysGlyLeuLeuLeuValIleAlaLeuGlyCysThrCysLysLeuTyrAlaIle
ArgThrGlnGluTyrSerIlePheAlaProLeuSerArgMetGluAlaGluIleValGlnGlnGlnAlaProProSerTyr
GlyGlnLeuIleAlaGlnGlyAlaIleProProValGluAspPheProThrGluAsnProAsnAspAsnSerValLeu
GlyAsnLeuArgSerLeuLeuGlnIleLeuArgGlnAspMetThrProGlyGlyGlyProGlyAlaArgArgArgGln
ArgGlyArgLeuMetArgArgLeuValArgArgLeuArgArgTrpGlyLeuLeuProArgThrAsnThrProAlaArg
AlaSerGluAlaArgSerGlnValThrProSerAlaAlaProLeuGluAlaLeuAspGlyGlyThrGlyProAlaArg
GluGlyGlyAlaValGlyGlyGlnAspGlyGluGlnAlaProProLeuProIleLysAlaProLeuProSerAlaSerThr
SerProAlaProThrThrValProGluAlaProGlyProLeuProSerLeuProLeuGluProSerLeuLeuSerGlyVal
ValGlnAlaLeuArgGlyArgLeuLeuProSerLeuGlyProProGlyProThrArgSerProProGlyProHisThr
AlaValLeuAlaLeuGluAspGluAspAspValLeuLeuValProLeuAlaGluProGlyValTrpValAlaGluAla
GluAspGluProLeuLeuThr
Protein: Osbp15

| | |
|---|---|
| <210> | 13 |
| <211> | 879 |
| <212> | PRT |
| <213> | Homo sapiens |
| <221> | WIPO Standard ST.26 |
| <222> | 1...879 |
| <400> | 13 |

MKEEAFLRRRFSLCPPSSTPQKVDPRKLTRNLLLSGDNELYPLSPGKDMEPNG
PSLPRDEGPPTPSSATKVPPAEYRLCNGSDKECVSPTARVTKKETLKAQKENYRQEKKR
ATRQLLSALTDPSVVIMADSLKIRGTLKSWTKLWCVLKPGVLLIYKTPKVGQWVGTVL
LHCCELIERPSKKDGFCFKLFHPLDQSVWAVKGPKGESVGSITQPLPSSYLIFRAASESDG
RCWLDALELALRCSSLLRLGTCKPGRDGEPGTSPDASPSSLCGLPASATVHPDQDLFPLN
GSSLENDAFSDKSERENPEESDTETQDHSRKTESGSDQSETPGAPVRRGTTYVEQVQEEL
GELGEASQVETVSEENKSLMWTLLKQLRPGMDLSRVVLPTFVLEPRSFLNKLSDYYYH
ADLLSRAAVEEDAYSRMKLVLRWYLSGFYKKPKGIKKPYNPILGETFRCCWFHPQTDS
RTFYIAEQVSHHPPVSAFHVSNRKDGFCISGSITAKSRFYGNSLSALLDGKATLTFLNRAE
DYTLTMPYAHCKGILYGTMTLELGGKVTIECAKNNFQAQLEFKLKPFFGGSTSINQISGK
ITSGEEVLASLSGHWDRDVFIKEEGSGSSALFWTPSGEVRRQRLRQHTVPLEEQTELESE
RLWQHVTRAISKGDQHRATQEKFALEEAQRQRARERQESLMPWKPQLFHLDPITQEWH
YRYEDHSPWDPLKDIAQFEQDGILRTLQQEAVARQTTFLGSPGPRHERSGPDQRLRKAS
DQPSGHSQATESSGSTPESCPELSDEEQDGDFVPGGESPCPRCRKEARRLQALHEAILSIR
EAQQELHRHLSAMLSSTARAAQAPTPGLLQSPRSWFLLCVFLACQLFINHILK
MetLysGluGluAlaPheLeuArgArgArgPheSerLeuCysProProSerSerThrProGlnLysVal
AspProArgLysLeuThrArgAsnLeuLeuLeuSerGlyAspAsnGluLeuTyrProLeuSerProGlyLysAsp
MetGluProAsnGlyProSerLeuProArgAspGluGlyProProThrProSerSerAlaThrLysValProProAlaGlu
TyrArgLeuCysAsnGlySerAspLysGluCysValSerProThrAlaArgValThrLysLysGluThrLeuLysAla
GlnLysGluAsnTyrArgGlnGluLysLysArgAlaThrArgGlnLeuLeuSerAlaLeuThrAspProSerValVal
IleMetAlaAspSerLeuLysIleArgGlyThrLeuLysSerTrpThrLysLeuTrpCysValLeuLysProGlyValLeu
LeuIleTyrLysThrProLysValGlyGlnTrpValGlyThrValLeuLeuHisCysCysGluLeuIleGluArgPro
SerLysLysAspGlyPheCysPheLysLeuPheHisProLeuAspGlnSerValTrpAlaValLysGlyProLysGlyGlu
SerValGlySerIleThrGlnProLeuProSerSerTyrLeuIlePheArgAlaAlaSerGluSerAspGlyArgCysTrp
LeuAspAlaLeuGluLeuAlaLeuArgCysSerSerLeuLeuArgLeuGlyThrCysLysProGlyArgAspGly
GluProGlyThrSerProAspAlaSerProSerSerLeuCysGlyLeuProAlaSerAlaThrValHisProAspGlnAsp
LeuPheProLeuAsnGlySerSerLeuGluAsnAspAlaPheSerAspLysSerGluArgGluAsnProGluGluSer
AspThrGluThrGlnAspHisSerArgLysThrGluSerGlySerAspGlnSerGluThrProGlyAlaProValArgArg
GlyThrThrTyrValGluGlnValGlnGluGluLeuGlyGluLeuGlyGluAlaSerGlnValGluThrValSerGlu
GluAsnLysSerLeuMetTrpThrLeuLeuLysGlnLeuArgProGlyMetAspLeuSerArgValValLeuProThrP
heValLeuGluProArgSerPheLeuAsnLysLeuSerAspTyrTyrTyrHisAlaAspLeuLeuSerArgAlaAla
ValGluGluAspAlaTyrSerArgMetLysLeuValLeuArgTrpTyrLeuSerGlyPheTyrLysLysProLysGlyIle
LysLysProTyrAsnProIleLeuGlyGluThrPheArgCysCysTrpPheHisProGlnThrAspSerArgThrPhe
TyrIleAlaGluGlnValSerHisHisProProValSerAlaPheHisValSerAsnArgLysAspGlyPheCysIleSer
GlySerIleThrAlaLysSerArgPheTyrGlyAsnSerLeuSerAlaLeuLeuAspGlyLysAlaThrLeuThrPheLeu
AsnArgAlaGluAspTyrThrLeuThrMetProTyrAlaHisCysLysGlyIleLeuTyrGlyThrMetThrLeuGlu
LeuGlyGlyLysValThrIleGluCysAlaLysAsnAsnPheGlnAlaGlnLeuGluPheLysLeuLysProPhePhe
GlyGlySerThrSerIleAsnGlnIleSerGlyLysIleThrSerGlyGluGluValLeuAlaSerLeuSerGlyHisTrp
AspArgAspValPheIleLysGluGluGlySerGlySerSerAlaLeuPheTrpThrProSerGlyGluValArgArgGln
ArgLeuArgGlnHisThrValProLeuGluGluGlnThrGluLeuGluSerGluArgLeuTrpGlnHisValThrArg
AlaIleSerLysGlyAspGlnHisArgAlaThrGlnGluLysPheAlaLeuGluGluAlaGlnArgGlnArgAlaArg
GluArgGlnGluSerLeuMetProTrpLysProGlnLeuPheHisLeuAspProIleThrGlnGluTrpHisTyrArg
TyrGluAspHisSerProTrpAspProLeuLysAspIleAlaGlnPheGluGlnAspGlyIleLeuArgThrLeuGlnGln
GluAlaValAlaArgGlnThrThrPheLeuGlySerProGlyProArgHisGluArgSerGlyProAspGlnArgLeu
ArgLysAlaSerAspGlnProSerGlyHisSerGlnAlaThrGluSerSerGlySerThrProGluSerCysProGluLeu
SerAspGluGluGlnAspGlyAspPheValProGlyGlyGluSerProCysProArgCysArgLysGluAlaArgArg
LeuGlnAlaLeuHisGluAlaIleLeuSerIleArgGluAlaGlnGlnGluLeuHisArgHisLeuSerAlaMetLeu
SerSerThrAlaArgAlaAlaGlnAlaProThrProGlyLeuLeuGlnSerProArgSerTrpPheLeuLeuCysValPhe
LeuAlaCysGlnLeuPheIleAsnHisIleLeuLys
Protein: Osbp11a

| | |
|---|---|
| <210> | 14 |
| <211> | 950 |
| <212> | PRT |
| <213> | Homo sapiens |
| <221> | WIPO Standard ST.26 |
| <222> | 1...950 |
| <400> | 14 |

MNTEAEQQLLHHARNGNAEEVRQLLETMARNEVIADINCKGRSKSNLGWTP
LHLACYFGHRQVVQDLLKAGAEVNVLNDMGDTPLHRAAFTGRKELVMLLLEYNADTT
IVNGSGQTAKEVTHAEEIRSMLEAVERTQQRKLEELLLAAAREGKTTELTALLNRPNPP
DVNCSDQLGNTPLHCAAYRAHKQCALKLLRSGADPNLKNKNDQKPLDLAQGAEMKHI
LVGNKVIYKALKRYEGPLWKSSRFFGWRLFWVVLEHGVLSWYRKQPDAVHNIYRQGC
KHLTQAVCTVKSTDSCLFFIKCFDDTIHGFRVPKNSLQQSREDWLEAIEEHSAYSTHYCS
QDQLTDEEEEDTVSAADLKKSLEKAQSCQQRLDREISNFLKMIKECDMAKEMLPSFLQK
VEVVSEASRETCVALTDCLNLFTKQEGVRNFKLEQEQEKNKILSEALETLATEHHELEQS
LVKGSPPASILSEDEFYDALSDSESERSLSRLEAVTARSFEEEGEHLGSRKHRMSEEKDC
GGGDALSNGIKKHRTSLPSPMFSRNDFSIWSILRKCIGMELSKITMPVIFNEPLSFLQRLTE
YMEHTYLIHKASSLSDPVERMQCVAAFAVSAVASQWERTGKPFNPLLGETYELVRDDL
GFRLISEQVSHHPPISAFHAEGLNNDFIFHGSIYPKLKFWGKSVEAEPKGTITLELLEHNE
AYTWTNPTCCVHNIIVGKLWIEQYGNVEIINHKTGDKCVLNFKPCGLFGKELHKVEGYI
QDKSKKKLCALYGKWTECLYSVDPATFDAYKKNDKKNTEEKKNSKOMSTSEELDEMP
VPDSESVFIIPGSVLLWRIAPRPPNSAQMYNFTSFAMVLNEVDKDMESVIPKTDCRLRPDI
RAMENGEIDQASEEKKRLEEKQRAARKNRSKSEEDWKTRWFHQGPNPYNGAQDWIYS
GSYWDRNYFNLPDIY
MetAsnThrGluAlaGluGlnGlnLeuLeuHisHisAlaArgAsnGlyAsnAlaGluGluValArg
GlnLeuLeuGluThrMetAlaArgAsnGluValIleAlaAspIleAsnCysLysGlyArgSerLysSerAsnLeuGlyTrp
ThrProLeuHisLeuAlaCysTyrPheGlyHisArgGlnValValGlnAspLeuLeuLysAlaGlyAlaGluValAsn
ValLeuAsnAspMetGlyAspThrProLeuHisArgAlaAlaPheThrGlyArgLysGluLeuValMetLeuLeu
LeuGluTyrAsnAlaAspThrThrIleValAsnGlySerGlyGlnThrAlaLysGluValThrHisAlaGluGluIleArg
SerMetLeuGluAlaValGluArgThrGlnGlnArgLysLeuGluGluLeuLeuLeuAlaAlaAlaArgGluGlyLys
ThrThrGluLeuThrAlaLeuLeuAsnArgProAsnProProAspValAsnCysSerAspGlnLeuGlyAsnThr
ProLeuHisCysAlaAlaTyrArgAlaHisLysGlnCysAlaLeuLysLeuLeuArgSerGlyAlaAspProAsnLeu
LysAsnLysAsnAspGlnLysProLeuAspLeuAlaGlnGlyAlaGluMetLysHisIleLeuValGlyAsnLysValIle
TyrLysAlaLeuLysArgTyrGluGlyProLeuTrpLysSerSerArgPhePheGlyTrpArgLeuPheTrpValVal
LeuGluHisGlyValLeuSerTrpTyrArgLysGlnProAspAlaValHisAsnIleTyrArgGlnGlyCysLysHis
LeuThrGlnAlaValCysThrValLysSerThrAspSerCysLeuPhePheIleLysCysPheAspAspThrIleHisGly
PheArgValProLysAsnSerLeuGlnGlnSerArgGluAspTrpLeuGluAlaIleGluGluHisSerAlaTyrSer
ThrHisTyrCysSerGlnAspGlnLeuThrAspGluGluGluGluAspThrValSerAlaAlaAspLeuLysLysSer
LeuGluLysAlaGlnSerCysGlnGlnArgLeuAspArgGluIleSerAsnPheLeuLysMetIleLysGluCysAsp
MetAlaLysGluMetLeuProSerPheLeuGlnLysValGluValValSerGluAlaSerArgGluThrCysValAlaLeu
ThrAspCysLeuAsnLeuPheThrLysGlnGluGlyValArgAsnPheLysLeuGluGlnGluGlnGluLysAsn
LysIleLeuSerGluAlaLeuGluThrLeuAlaThrGluHisHisGluLeuGluGlnSerLeuValLysGlySerProPro
AlaSerIleLeuSerGluAspGluPheTyrAspAlaLeuSerAspSerGluSerGluArgSerLeuSerArgLeuGlu
AlaValThrAlaArgSerPheGluGluGluGlyGluHisLeuGlySerArgLysHisArgMetSerGluGluLysAsp
CysGlyGlyGlyAspAlaLeuSerAsnGlyIleLysLysHisArgThrSerLeuProSerProMetPheSerArgAsnAsp
PheSerIleTrpSerIleLeuArgLysCysIleGlyMetGluLeuSerLysIleThrMetProValIlePheAsnGluPro
LeuSerPheLeuGlnArgLeuThrGluTyrMetGluHisThrTyrLeuIleHisLysAlaSerSerLeuSerAspProVal
GluArgMetGlnCysValAlaAlaPheAlaValSerAlaValAlaSerGlnTrpGluArgThrGlyLysProPheAsn
ProLeuLeuGlyGluThrTyrGluLeuValArgAspAspLeuGlyPheArgLeuIleSerGluGlnValSerHisHisPro
ProIleSerAlaPheHisAlaGluGlyLeuAsnAsnAspPheIlePheHisGlySerIleTyrProLysLeuLysPheTrp
GlyLysSerValGluAlaGluProLysGlyThrIleThrLeuGluLeuLeuGluHisAsnGluAlaTyrThrTrpThr
AsnProThrCysCysValHisAsnIleIleValGlyLysLeuTrpIleGluGlnTyrGlyAsnValGluIleIleAsnHisLys
ThrGlyAspLysCysValLeuAsnPheLysProCysGlyLeuPheGlyLysGluLeuHisLysValGluGlyTyrIle
GlnAspLysSerLysLysLysLeuCysAlaLeuTyrGlyLysTrpThrGluCysLeuTyrSerValAspProAlaThr
PheAspAlaTyrLysLysAsnAspLysLysAsnThrGluGluLysLysAsnSerLysGlnMetSerThrSerGluGlu
LeuAspGluMetProValProAspSerGluSerValPheIleIleProGlySerValLeuLeuTrpArgIleAlaProArg
ProProAsnSerAlaGlnMetTyrAsnPheThrSerPheAlaMetValLeuAsnGluValAspLysAspMetGluSer
ValIleProLysThrAspCysArgLeuArgProAspIleArgAlaMetGluAsnGlyGluIleAspGlnAlaSerGlu
GluLysLysArgLeuGluGluLysGlnArgAlaAlaArgLysAsnArgSerLysSerGluGluAspTrpLysThrArg
TrpPheHisGlnGlyProAsnProTyrAsnGlyAlaGlnAspTrpIleTyrSerGlySerTyrTrpAspArgAsnTyrPhe
AsnLeuProAspIleTyr
Protein: Prkag2

| | |
|---|---|
| <210> | 15 |
| <211> | 569 |
| <212> | PRT |
| <213> | Homo sapiens |
| <221> | WIPO Standard ST.26 |
| <222> | 1...569 |
| <400> | 15 |

MGSAVMDTKKKKDVSSPGGSGGKKNASQKRRSLRVHIPDLSSFAMPLLDGD
LEGSGKHSSRKVDSPFGPGSPSKGFFSRGPQPRPSSPMSAPVRPKTSPGSPKTVFPFSYQE
SPPRSPRRMSFSGIFRSSSKESSPNSNPATSPGGIRFFSRSRKTSGLSSSPSTPTQVTKQHTF
PLESYKHEPERLENRIYASSSPPDTGQRFCPSSFQSPTRPPLASPTHYAPSKAAALAAALG
PAEAGMLEKLEFEDEAVEDSESGVYMRFMRSHKCYDIVPTSSKLVVFDTTLQVKKAFF
ALVANGVRAAPLWESKKQSFVGMLTITDFINILHRYYKSPMVQIYELEEHKIETWRELY
LQETFKPLVNISPDASLFDAVYSLIKNKIHRLPVIDPISGNALYILTHKRILKFLQLFMSDM
PKPAFMKQNLDELGIGTYHNIAFIHPDTPIIKALNIFVERRISALPVVDESGKVVDIYSKFD
VINLAAEKTYNNLDITVTQALQHRSQYFEGVVKCNKLEILETIVDRIVRAEVHRLVVVNE
ADSIVGIISLSDILQALILTPAGAKQKETETE
MetGlySerAlaValMetAspThrLysLysLysLysAspValSerSerProGlyGlySerGlyGlyLys
LysAsnAlaSerGlnLysArgArgSerLeuArgValHisIleProAspLeuSerSerPheAlaMetProLeuLeuAsp
GlyAspLeuGluGlySerGlyLysHisSerSerArgLysValAspSerProPheGlyProGlySerProSerLysGlyPhe
PheSerArgGlyProGlnProArgProSerSerProMetSerAlaProValArgProLysThrSerProGlySerProLys
ThrValPheProPheSerTyrGlnGluSerProProArgSerProArgArgMetSerPheSerGlyIlePheArgSerSer
SerLysGluSerSerProAsnSerAsnProAlaThrSerProGlyGlyIleArgPhePheSerArgSerArgLysThrSer
GlyLeuSerSerSerProSerThrProThrGlnValThrLysGlnHisThrPheProLeuGluSerTyrLysHisGluPro
GluArgLeuGluAsnArgIleTyrAlaSerSerSerProProAspThrGlyGlnArgPheCysProSerSerPheGln
SerProThrArgProProLeuAlaSerProThrHisTyrAlaProSerLysAlaAlaAlaLeuAlaAlaAlaLeuGlyPro
AlaGluAlaGlyMetLeuGluLysLeuGluPheGluAspGluAlaValGluAspSerGluSerGlyValTyrMetArg
PheMetArgSerHisLysCysTyrAspIleValProThrSerSerLysLeuValValPheAspThrThrLeuGlnValLys
LysAlaPhePheAlaLeuValAlaAsnGlyValArgAlaAlaProLeuTrpGluSerLysLysGlnSerPheValGly
MetLeuThrIleThrAspPheIleAsnIleLeuHisArgTyrTyrLysSerProMetValGlnIleTyrGluLeuGluGlu
HisLysIleGluThrTrpArgGluLeuTyrLeuGlnGluThrPheLysProLeuValAsnIleSerProAspAlaSerLeu
PheAspAlaValTyrSerLeuIleLysAsnLysIleHisArgLeuProValIleAspProIleSerGlyAsnAlaLeuTyrIle
LeuThrHisLysArgIleLeuLysPheLeuGlnLeuPheMetSerAspMetProLysProAlaPheMetLysGlnAsn
LeuAspGluLeuGlyIleGlyThrTyrHisAsnIleAlaPheIleHisProAspThrProIleIleLysAlaLeuAsnIle
PheValGluArgArgIleSerAlaLeuProValValAspGluSerGlyLysValValAspIleTyrSerLysPheAspVal
IleAsnLeuAlaAlaGluLysThrTyrAsnAsnLeuAspIleThrValThrGlnAlaLeuGlnHisArgSerGlnTyrPhe
GluGlyValValLysCysAsnLysLeuGluIleLeuGluThrIleValAspArgIleValArgAlaGluValHisArg
LeuValValValAsnGluAlaAspSerIleValGlyIleIleSerLeuSerAspIleLeuGlnAlaLeuIleLeuThrProAla
GlyAlaLysGlnLysGluThrGluThrGlu
Protein: Mvk

| | |
|---|---|
| <210> | 16 |
| <211> | 396 |
| <212> | PRT |
| <213> | Homo sapiens |
| <221> | WIPO Standard ST.26 |
| <222> | 1...396 |
| <400> | 16 |

MLSEVLLVSAPGKVILHGEHAVVHGKVALAVSLNLRTFLRLQPHSNGKVDLS
LPNIGIKRAWDVARLQSLDTSFLEQGDVTTPTSEQVEKLKEVAGLPDDCAVTERLAVLA
FLYLYLSICRKQRALPSLDIVVWSELPPGAGLGSSAAYSVCLAAALLTVCEEIPNPLKDG
DCVNRWTKEDLELINKWAFQGERMIHGNPSGVDNAVSTWGGALRYHQGKISSLKRSPA
LQILLTNTKVPRNTRALVAGVRNRLLKFPEIVAPLLTSIDAISLECERVLGEMGEAPAPEQ
YLVLEELIDMNQHHLNALGVGHASLDQLCQVTRARGLHSKLTGAGGGGCGITLLKPGL
EQPEVEATKQALTSCGFDCLETSIGAPGVSIHSATSLDSRVQQALDGL
MetLeuSerGluValLeuLeuValSerAlaProGlyLysValIleLeuHisGlyGluHisAlaValVal
HisGlyLysValAlaLeuAlaValSerLeuAsnLeuArgThrPheLeuArgLeuGlnProHisSerAsnGlyLysVal
AspLeuSerLeuProAsnIleGlyIleLysArgAlaTrpAspValAlaArgLeuGlnSerLeuAspThrSerPheLeuGlu
GlnGlyAspValThrThrProThrSerGluGlnValGluLysLeuLysGluValAlaGlyLeuProAspAspCysAla
ValThrGluArgLeuAlaValLeuAlaPheLeuTyrLeuTyrLeuSerIleCysArgLysGlnArgAlaLeuProSer
LeuAspIleValValTrpSerGluLeuProProGlyAlaGlyLeuGlySerSerAlaAlaTyrSerValCysLeuAlaAla
AlaLeuLeuThrValCysGluGluIleProAsnProLeuLysAspGlyAspCysValAsnArgTrpThrLysGluAsp
LeuGluLeuIleAsnLysTrpAlaPheGlnGlyGluArgMetIleHisGlyAsnProSerGlyValAspAsnAlaVal
SerThrTrpGlyGlyAlaLeuArgTyrHisGlnGlyLysIleSerSerLeuLysArgSerProAlaLeuGlnIleLeuLeu
ThrAsnThrLysValProArgAsnThrArgAlaLeuValAlaGlyValArgAsnArgLeuLeuLysPheProGluIle
ValAlaProLeuLeuThrSerIleAspAlaIleSerLeuGluCysGluArgValLeuGlyGluMetGlyGluAlaProAla
ProGluGlnTyrLeuValLeuGluGluLeuIleAspMetAsnGlnHisHisLeuAsnAlaLeuGlyValGlyHisAla
SerLeuAspGlnLeuCysGlnValThrArgAlaArgGlyLeuHisSerLysLeuThrGlyAlaGlyGlyGlyGlyCys
GlyIleThrLeuLeuLysProGlyLeuGluGlnProGluValGluAlaThrLysGlnAlaLeuThrSerCysGlyPhe
AspCysLeuGluThrSerIleGlyAlaProGlyValSerIleHisSerAlaThrSerLeuAspSerArgValGlnGlnAla
LeuAspGlyLeu
Protein: Serpinb2

| | |
|---|---|
| <210> | 17 |
| <211> | 415 |
| <212> | PRT |
| <213> | Homo sapiens |
| <221> | WIPO Standard ST.26 |
| <222> | 1...415 |
| <400> | 17 |

MEDLCVANTLFALNLFKHLAKASPTQNLFLSPWSISSTMAMVYMGSRGSTE
DQMAKVLQFNEVGANAVTPMTPENFTSCGFMQQIQKGSYPDAILQAQAADKIHSSFRSL
SSAINASTGNYLLESVNKLFGEKSASFREEYIRLCQKYYSSEPQAVDFLECAEEARKKINS
WVKTQTKGKIPNLLPEGSVDGDTRMVLVNAVYFKGKWKTPFEKKLNGLYPFRVNSAQ
RTPVQMMYLREKLNIGYIEDLKAQILELPYAGDVSMFLLLPDEIADVSTGLELLESEITYD
KLNKWTSKDKMAEDEVEVYIPQFKLEEHYELRSILRSMGMEDAFNKGRANFSGMSERN
DLFLSEVFHQAMVDVNEEGTEAAAGTGGVMTGRTGHGGPQFVADHPFLFLIMHKITNC
ILFFGRFSSP
MetGluAspLeuCysValAlaAsnThrLeuPheAlaLeuAsnLeuPheLysHisLeuAlaLysAlaSe
rProThrGlnAsnLeuPheLeuSerProTrpSerIleSerSerThrMetAlaMetValTyrMetGlySerArgGlySer
ThrGluAspGlnMetAlaLysValLeuGlnPheAsnGluValGlyAlaAsnAlaValThrProMetThrProGluAsn
PheThrSerCysGlyPheMetGlnGlnIleGlnLysGlySerTyrProAspAlaIleLeuGlnAlaGlnAlaAlaAspLys
IleHisSerSerPheArgSerLeuSerSerAlaIleAsnAlaSerThrGlyAsnTyrLeuLeuGluSerValAsnLysLeu
PheGlyGluLysSerAlaSerPheArgGluGluTyrIleArgLeuCysGlnLysTyrTyrSerSerGluProGlnAla
ValAspPheLeuGluCysAlaGluGluAlaArgLysLysIleAsnSerTrpValLysThrGlnThrLysGlyLysIlePro
AsnLeuLeuProGluGlySerValAspGlyAspThrArgMetValLeuValAsnAlaValTyrPheLysGlyLysTrp
LysThrProPheGluLysLysLeuAsnGlyLeuTyrProPheArgValAsnSerAlaGlnArgThrProValGlnMet
MetTyrLeuArgGluLysLeuAsnIleGlyTyrIleGluAspLeuLysAlaGlnIleLeuGluLeuProTyrAlaGlyAsp
ValSerMetPheLeuLeuLeuProAspGluIleAlaAspValSerThrGlyLeuGluLeuLeuGluSerGluIleThr
TyrAspLysLeuAsnLysTrpThrSerLysAspLysMetAlaGluAspGluValGluValTyrIleProGlnPheLysLeu
GluGluHisTyrGluLeuArgSerIleLeuArgSerMetGlyMetGluAspAlaPheAsnLysGlyArgAlaAsnPhe
SerGlyMetSerGluArgAsnAspLeuPheLeuSerGluValPheHisGlnAlaMetValAspValAsnGluGluGly
ThrGluAlaAlaAlaGlyThrGlyGlyValMetThrGlyArgThrGlyHisGlyGlyProGlnPheValAlaAspHis
ProPheLeuPheLeuIleMetHisLysIleThrAsnCysIleLeuPhePheGlyArgPheSerSerPro
Protein: Pik3ca

| | |
|---|---|
| <210> | 18 |
| <211> | 1068 |
| <212> | PRT |
| <213> | Homo sapiens |
| <221> | WIPO Standard ST.26 |
| <222> | 1...1068 |
| <400> | 18 |

MPPRPSSGELWGIHLMPPRILVECLLPNGMIVTLECLREATLITIKHELFKEAR
KYPLHQLLQDESSYIFVSVTQEAEREEFFDETRRLCDLRLFQPFLKVIEPVGNREEKILNR
EIGFAIGMPVCEFDMVKDPEVQDFRRNILNVCKEAVDLRDLNSPHSRAMYVYPPNVESS
PELPKHIYNKLDKGQIIVVIWVIVSPNNDKQKYTLKINHDCVPEQVIAEAIRKKTRSMLLS
SEQLKLCVLEYQGKYILKVCGCDEYFLEKYPLSQYKYIRSCIMLGRMPNLMLMAKESLY
SQLPMDCFTMPSYSRRISTATPYMNGETSTKSLWVINSALRIKILCATYVNVNIRDIDKIY
VRTGIYHGGEPLCDNVNTQRVPCSNPRWNEWLNYDIYIPDLPRAARLCLSICSVKGRKG
AKEEHCPLAWGNINLFDYTDTLVSGKMALNLWPVPHGLEDLLNPIGVTGSNPNKETPCL
ELEFDWFSSVVKFPDMSVIEEHANWSVSREAGFSYSHAGLSNRLARDNELRENDKEQL
KAISTRDPLSEITEQEKDFLWSHRHYCVTIPEILPKLLLSVKWNSRDEVAQMYCLVKDWP
PIKPEQAMELLDCNYPDPMVRGFAVRCLEKYLTDDKLSQYLIQLVQVLKYEQYLDNLL
VRFLLKKALTNQRIGHFFFWHLKSEMHNKTVSQRFGLLLESYCRACGMYLKHLNRQVE
AMEKLINLTDILKQEKKDETQKVQMKFLVEQMRRPDFMDALQGFLSPLNPAHQLGNLR
LEECRIMSSAKRPLWLNWENPDIMSELLFQNNEIIFKNGDDLRQDMLTLQIIRIMENIWQ
NQGLDLRMLPYGCLSIGDCVGLIEVVRNSHTIMQIQCKGGLKGALQFNSHTLHQWLKD
KNKGEIYDAAIDLFTRSCAGYCVATFILGIGDRHNSNIMVKDDGQLFHIDFGHFLDHKKK
KFGYKRERVPFVLTQDFLIVISKGAQECTKTREFERFQEMCYKAYLAIRQHANLFINLES
MMLGSGMPELQSFDDIAYIRKTLALDKTEQEALEYFMKQMNDAHHGGWTTKMDWIFH
TIKQHALN
MetProProArgProSerSerGlyGluLeuTrpGlyIleHisLeuMetProProArgIleLeuValGlu
CysLeuLeuProAsnGlyMetIleValThrLeuGluCysLeuArgGluAlaThrLeuIleThrIleLysHisGluLeuPhe
LysGluAlaArgLysTyrProLeuHisGlnLeuLeuGlnAspGluSerSerTyrIlePheValSerValThrGlnGluAla
GluArgGluGluPhePheAspGluThrArgArgLeuCysAspLeuArgLeuPheGlnProPheLeuLysValIle
GluProValGlyAsnArgGluGluLysIleLeuAsnArgGluIleGlyPheAlaIleGlyMetProValCysGluPheAsp
MetValLysAspProGluValGlnAspPheArgArgAsnIleLeuAsnValCysLysGluAlaValAspLeuArg
AspLeuAsnSerProHisSerArgAlaMetTyrValTyrProProAsnValGluSerSerProGluLeuProLysHisIle
TyrAsnLysLeuAspLysGlyGlnIleIleValValIleTrpValIleValSerProAsnAsnAspLysGlnLysTyrThr
LeuLysIleAsnHisAspCysValProGluGlnValIleAlaGluAlaIleArgLysLysThrArgSerMetLeuLeuSer
SerGluGlnLeuLysLeuCysValLeuGluTyrGlnGlyLysTyrIleLeuLysValCysGlyCysAspGluTyrPhe
LeuGluLysTyrProLeuSerGlnTyrLysTyrIleArgSerCysIleMetLeuGlyArgMetProAsnLeuMetLeuMet
AlaLysGluSerLeuTyrSerGlnLeuProMetAspCysPheThrMetProSerTyrSerArgArgIleSerThrAla
ThrProTyrMetAsnGlyGluThrSerThrLysSerLeuTrpValIleAsnSerAlaLeuArgIleLysIleLeuCysAla
ThrTyrValAsnValAsnIleArgAspIleAspLysIleTyrValArgThrGlyIleTyrHisGlyGlyGluProLeuCys
AspAsnValAsnThrGlnArgValProCysSerAsnProArgTrpAsnGluTrpLeuAsnTyrAspIleTyrIleProAsp
LeuProArgAlaAlaArgLeuCysLeuSerIleCysSerValLysGlyArgLysGlyAlaLysGluGluHisCysPro
LeuAlaTrpGlyAsnIleAsnLeuPheAspTyrThrAspThrLeuValSerGlyLysMetAlaLeuAsnLeuTrpPro
ValProHisGlyLeuGluAspLeuLeuAsnProIleGlyValThrGlySerAsnProAsnLysGluThrProCysLeu
GluLeuGluPheAspTrpPheSerSerValValLysPheProAspMetSerValIleGluGluHisAlaAsnTrpSerVal
SerArgGluAlaGlyPheSerTyrSerHisAlaGlyLeuSerAsnArgLeuAlaArgAspAsnGluLeuArgGluAsn
AspLysGluGlnLeuLysAlaIleSerThrArgAspProLeuSerGluIleThrGluGlnGluLysAspPheLeuTrpSer
HisArgHisTyrCysValThrIleProGluIleLeuProLysLeuLeuLeuSerValLysTrpAsnSerArgAspGlu
ValAlaGlnMetTyrCysLeuValLysAspTrpProProIleLysProGluGlnAlaMetGluLeuLeuAspCysAsnTyr
ProAspProMetValArgGlyPheAlaValArgCysLeuGluLysTyrLeuThrAspAspLysLeuSerGlnTyr
LeuIleGlnLeuValGlnValLeuLysTyrGluGlnTyrLeuAspAsnLeuLeuValArgPheLeuLeuLysLysAla
LeuThrAsnGlnArgIleGlyHisPhePhePheTrpHisLeuLysSerGluMetHisAsnLysThrValSerGlnArgPhe
GlyLeuLeuLeuGluSerTyrCysArgAlaCysGlyMetTyrLeuLysHisLeuAsnArgGlnValGluAlaMet
GluLysLeuIleAsnLeuThrAspIleLeuLysGlnGluLysLysAspGluThrGlnLysValGlnMetLysPheLeu
ValGluGlnMetArgArgProAspPheMetAspAlaLeuGlnGlyPheLeuSerProLeuAsnProAlaHisGlnLeu
GlyAsnLeuArgLeuGluGluCysArgIleMetSerSerAlaLysArgProLeuTrpLeuAsnTrpGluAsnProAspIle
MetSerGluLeuLeuPheGlnAsnAsnGluIleIlePheLysAsnGlyAspAspLeuArgGlnAspMetLeuThr
LeuGlnIleIleArgIleMetGluAsnIleTrpGlnAsnGlnGlyLeuAspLeuArgMetLeuProTyrGlyCysLeuSer
IleGlyAspCysValGlyLeuIleGluValValArgAsnSerHisThrIleMetGlnIleGlnCysLysGlyGlyLeuLys
GlyAlaLeuGlnPheAsnSerHisThrLeuHisGlnTrpLeuLysAspLysAsnLysGlyGluIleTyrAspAlaAlaIle
AspLeuPheThrArgSerCysAlaGlyTyrCysValAlaThrPheIleLeuGlyIleGlyAspArgHisAsnSerAsn
IleMetValLysAspAspGlyGlnLeuPheHisIleAspPheGlyHisPheLeuAspHisLysLysLysLysPheGly
TyrLysArgGluArgValProPheValLeuThrGlnAspPheLeuIleValIleSerLysGlyAlaGlnGluCysThrLys
ThrArgGluPheGluArgPheGlnGluMetCysTyrLysAlaTyrLeuAlaIleArgGlnHisAlaAsnLeuPheIle
AsnLeuPheSerMetMetLeuGlySerGlyMetProGluLeuGlnSerPheAspAspIleAlaTyrIleArgLysThrLeu
AlaLeuAspLysThrGluGlnGluAlaLeuGluTyrPheMetLysGlnMetAsnAspAlaHisHisGlyGlyTrp
ThrThrLysMetAspTrpIlePheHisThrIleLysGlnHisAlaLeuAsn
Protein: Pik3cd

| | |
|---|---|
| <210> | 19 |
| <211> | 1044 |
| <212> | PRT |
| <213> | Homo sapiens |
| <221> | WIPO Standard ST.26 |
| <222> | 1...1044 |
| <400> | 19 |

MPPGVDCPMEFWTKEENQSVVVDFLLPTGVYLNFPVSRNANLSTIKQLLWH
RAQYEPLFHMLSGPEAYVFTCINQTAEQQELEDEQRRLCDVQPFLPVLRLVAREGDRVK
KLINSQISLLIGKGLHEFDSLCDPEVNDFRAKMCQFCEEAAARRQQLGWEAWLQYSFPL
QLEPSAQTWGPGTLRLPNRALLVNVKFEGSEESFTFQVSTKDVPLALMACALRKKATVF
RQPLVEQPEDYTLQVNGRHEYLYGSYPLCQFQYICSCLHSGLTPHLTMVHSSSILAMRD
EQSNPAPQVQKPRAKPPPIPAKKPSSVSLWSLEQPFRIELIQGSKVNADERMKLVVQAGL
FHGNEMLCKTVSSSEVSVCSEPVWKQRLEFDINICDLPRMARLCFALYAVIEKAKKARS
TKKKSKKADCPIAWANLMLFDYKDQLKTGERCLYMWPSVPDEKGELLNPTGTVRSNP
NTDSAAALLICLPEVAPHPVYYPALEKILELGRHSECVHVTEEEQLQLREILERRGSGELY
EHEKDLVWKLRHEVQEHFPEALARLLLVTKWNKHEDVAQMLYLLCSWPELPVLSALE
LLDFSFPDCHVGSFAIKSLRKLTDDELFQYLLQLVQVLKYESYLDCELTKFLLDRALANR
KIGHFLFWHLRSEMHVPSVALRFGLILEAYCRGSTHHMKVLMKQGEALSKLKALNDFV
KLSSQKTPKPQTKELMHLCMRQEAYLEALSHLQSPLDPSTLLAEVCVEQCTFMDSKMK
PLWIMYSNEEAGSGGSVGIIFKNGDDLRQDMLTLQMIQLMDVLWKQEGLDLRMTPYGC
LPTGDRTGLIEVVLRSDTIANIQLNKSNMAATAAFNKDALLNWLKSKNPGEALDRAIEE
FTLSCAGYCVATYVLGIGDRHSDNIMIRESGQLFHIDFGHFLGNFKTKFGINRERVPFILT
YDFVHVIQQGKTNNSEKFERFRGYCERAYTILRRHGLLFLHLFALMRAAGLPELSCSKDI
QYLKDSLALGKTEEEALKHFRVKFNEALRESWKTKVNWLAHNVSKDNRQ
MetProProGlyValAspCysProMetGluPheTrpThrLysGluGluAsnGlnSerValValValAsp
PheLeuLeuProThrGlyValTyrLeuAsnPheProValSerArgAsnAlaAsnLeuSerThrIleLysGlnLeuLeu
TrpHisArgAlaGlnTyrGluProLeuPheHisMetLeuSerGlyProGluAlaTyrValPheThrCysIleAsnGlnThr
AlaGluGlnGlnGluLeuGluAspGluGlnArgArgLeuCysAspValGlnProPheLeuProValLeuArgLeu
ValAlaArgGluGlyAspArgValLysLysLeuIleAsnSerGlnIleSerLeuLeuIleGlyLysGlyLeuHisGluPhe
AspSerLeuCysAspProGluValAsnAspPheArgAlaLysMetCysGlnPheCysGluGluAlaAlaAlaArgArg
GlnGlnLeuGlyTrpGluAlaTrpLeuGlnTyrSerPheProLeuGlnLeuGluProSerAlaGlnThrTrpGlyPro
GlyThrLeuArgLeuProAsnArgAlaLeuLeuValAsnValLysPheGluGlySerGluGluSerPheThrPheGln
ValSerThrLysAspValProLeuAlaLeuMetAlaCysAlaLeuArgLysLysAlaThrValPheArgGlnProLeu
ValGluGlnProGluAspTyrThrLeuGlnValAsnGlyArgHisGluTyrLeuTyrGlySerTyrProLeuCysGln
PheGlnTyrIleCysSerCysLeuHisSerGlyLeuThrProHisLeuThrMetValHisSerSerSerIleLeuAlaMet
ArgAspGluGlnSerAsnProAlaProGlnValGlnLysProArgAlaLysProProProIleProAlaLysLysProSer
SerValSerLeuTrpSerLeuGluGlnProPheArgIleGluLeuIleGlnGlySerLysValAsnAlaAspGluArgMet
LysLeuValValGlnAlaGlyLeuPheHisGlyAsnGluMetLeuCysLysThrValSerSerSerGluValSerVal
CysSerGluProValTrpLysGlnArgLeuGluPheAspIleAsnIleCysAspLeuProArgMetAlaArgLeuCysPhe
AlaLeuTyrAlaValIleGluLysAlaLysLysAlaArgSerThrLysLysLysSerLysLysAlaAspCysProIleAla
TrpAlaAsnLeuMetLeuPheAspTyrLysAspGlnLeuLysThrGlyGluArgCysLeuTyrMetTrpProSer
ValProAspGluLysGlyGluLeuLeuAsnProThrGlyThrValArgSerAsnProAsnThrAspSerAlaAlaAlaLeu
LeuIleCysLeuProGluValAlaProHisProValTyrTyrProAlaLeuGluLysIleLeuGluLeuGlyArgHisSer
GluCysValHisValThrGluGluGluGlnLeuGlnLeuArgGluIleLeuGluArgArgGlySerGlyGluLeuTyr
GluHisGluLysAspLeuValTrpLysLeuArgHisGluValGlnGluHisPheProGluAlaLeuAlaArgLeuLeu
LeuValThrLysTrpAsnLysHisGluAspValAlaGlnMetLeuTyrLeuLeuCysSerTrpProGluLeuProVal
LeuSerAlaLeuGluLeuLeuAspPheSerPheProAspCysHisValGlySerPheAlaIleLysSerLeuArgLys
LeuThrAspAspGluLeuPheGlnTyrLeuLeuGlnLeuValGlnValLeuLysTyrGluSerTyrLeuAspCysGlu
LeuThrLysPheLeuLeuAspArgAlaLeuAlaAsnArgLysIleGlyHisPheLeuPheTrpHisLeuArgSerGlu
MetHisValProSerValAlaLeuArgPheGlyLeuIleLeuGluAlaTyrCysArgGlySerThrHisHisMetLys
ValLeuMetLysGlnGlyGluAlaLeuSerLysLeuLysAlaLeuAsnAspPheValLysLeuSerSerGlnLysThr
ProLysProGlnThrLysGluLeuMetHisLeuCysMetArgGlnGluAlaTyrLeuGluAlaLeuSerHisLeuGln
SerProLeuAspProSerThrLeuLeuAlaGluValCysValGluGlnCysThrPheMetAspSerLysMetLysPro
LeuTrpIleMetTyrSerAsnGluGluAlaGlySerGlyGlySerValGlyIleIlePheLysAsnGlyAspAspLeuArg
GlnAspMetLeuThrLeuGlnMetIleGlnLeuMetAspValLeuTrpLysGlnGluGlyLeuAspLeuArgMetThr
ProTyrGlyCysLeuProThrGlyAspArgThrGlyLeuIleGluValValLeuArgSerAspThrIleAlaAsnIle
GlnLeuAsnLysSerAsnMetAlaAlaThrAlaAlaPheAsnLysAspAlaLeuLeuAsnTrpLeuLysSerLysAsn
ProGlyGluAlaLeuAspArgAlaIleGluGluPheThrLeuSerCysAlaGlyTyrCysValAlaThrTyrValLeu
GlyIleGlyAspArgHisSerAspAsnIleMetIleArgGluSerGlyGlnLeuPheHisIleAspPheGlyHisPheLeu
GlyAsnPheLysThrLysPheGlyIleAsnArgGluArgValProPheIleLeuThrTyrAspPheValHisValIleGln
GlnGlyLysThrAsnAsnSerGluLysPheGluArgPheArgGlyTyrCysGluArgAlaTyrThrIleLeuArgArg
HisGlyLeuLeuPheLeuHisLeuPheAlaLeuMetArgAlaAlaGlyLeuProGluLeuSerCysSerLysAspIleGln
TyrLeuLysAspSerLeuAlaLeuGlyLysThrGluGluGluAlaLeuLysHisPheArgValLysPheAsnGluAla
LeuArgGluSerTrpLysThrLysValAsnTrpLeuAlaHisAsnValSerLysAspAsnArgGln
Protein: PIK3R3

| | |
|---|---|
| <210> | 20 |
| <211> | 461 |
| <212> | PRT |
| <213> | Homo sapiens |
| <221> | WIPO Standard ST.26 |
| <222> | 1...461 |
| <400> | 20 |

MYNTVWSMDRDDADWREVMMPYSTELIFYIEMDPPALPPKPPKPMTSAVPN
GMKDSSVSLQDAEWYWGDISREEVNDKLRDMPDGTFLVRDASTKMQGDYTLTLRKGG
NNKLIKIYHRDGKYGFSDPLTENSVVELINHYHHESLAQYNPKLDVKLMYPVSRYQQD
QLVKEDNIDAVGKKLQEYHSQYQEKSKEYDRLYEEYTRTSQEIQMKRTAIEAFNETIKIF
EEQCHTQEQHSKEYIERFRREGNEKEIERIMMNYDKLKSRLGEIHDSKMRLEQDLKNQA
LDNREIDKKMNSIKPDLIQLRKIRDQHLVWLNHKGVRQKRLNVWLGIKNEDADENYFIN
EEDENLPHYDEKTWFVEDINRVQAEDLLYGKPDGAFLIRESSKKGCYACSVVADGEVK
HCVIYSTARGYGFAEPYNLYSSLKELVLHYQQTSLVQHNDSLNVRLAYPVHAQMPSLC
R
MetTyrAsnThrValTrpSerMetAspArgAspAspAlaAspTrpArgGluValMetMetProTyr
SerThrGluLeuIlePheTyrIleGluMetAspProProAlaLeuProProLysProProLysProMetThrSerAlaValPro
AsnGlyMetLysAspSerSerValSerLeuGlnAspAlaGluTrpTyrTrpGlyAspIleSerArgGluGluValAsn
AspLysLeuArgAspMetProAspGlyThrPheLeuValArgAspAlaSerThrLysMetGlnGlyAspTyrThr
LeuThrLeuArgLysGlyGlyAsnAsnLysLeuIleLysIleTyrHisArgAspGlyLysTyrGlyPheSerAspProLeu
ThrPheAsnSerValValGluLeuIleAsnHisTyrHisHisGluSerLeuAlaGlnTyrAsnProLysLeuAspVal
LysLeuMetTyrProValSerArgTyrGlnGlnAspGlnLeuValLysGluAspAsnIleAspAlaValGlyLysLys
LeuGlnGluTyrHisSerGlnTyrGlnGluLysSerLysGluTyrAspArgLeuTyrGluGluTyrThrArgThrSer
GlnGluIleGlnMetLysArgThrAlaIleGluAlaPheAsnGluThrIleLysIlePheGluGluGlnCysHisThrGln
GluGlnHisSerLysGluTyrIleGluArgPheArgArgGluGlyAsnGluLysGluIleGluArgIleMetMetAsnTyr
AspLysLeuLysSerArgLeuGlyGluIleHisAspSerLysMetArgLeuGluGlnAspLeuLysAsnGlnAlaLeu
AspAsnArgGluIleAspLysLysMetAsnSerIleLysProAspLeuIleGlnLeuArgLysIleArgAspGlnHis
LeuValTrpLeuAsnHisLysGlyValArgGlnLysArgLeuAsnValTrpLeuGlyIleLysAsnGluAspAlaAsp
GluAsnTyrPheIleAsnGluGluAspGluAsnLeuProHisTyrAspGluLysThrTrpPheValGluAspIleAsnArg
ValGlnAlaGluAspLeuLeuTyrGlyLysProAspGlyAlaPheLeuIleArgGluSerSerLysLysGlyCysTyr
AlaCysSerValValAlaAspGlyGluValLysHisCysValIleTyrSerThrAlaArgGlyTyrGlyPheAlaGlu
ProTyrAsnLeuTyrSerSerLeuLysGluLeuValLeuHisTyrGlnGlnThrSerLeuValGlnHisAsnAspSerLeu
AsnValArgLeuAlaTyrProValHisAlaGlnMetProSerLeuCysArg
Protein: GLS2

| | |
|---|---|
| <210> | 21 |
| <211> | 602 |
| <212> | PRT |
| <213> | Homo sapiens |
| <221> | WIPO Standard ST.26 |
| <222> | 1...602 |
| <400> | 21 |

MRSMKALQKALSRAGSHCGRGGWGHPSRSPLLGGGVRHHLSEAAAQGRET
PHSHQPQHQDHDSSESGMLSRLGDLLFYTIAEGQERIPIHKFTTALKATGLQTSDPRLRD
CMSEMHRVVQESSSGGLLDRDLFRKCVSSNIVLLTQAFRKKFVIPDFEEFTGHVDRIFED
VKELTGGKVAAYIPQLAKSNPDLWGVSLCTVDGQRHSVGHTKIPFCLQSCVKPLTYAISI
STLGTDYVHKFVGKEPSGLRYNKLSLNEEGIPHNPMVNAGAIVVSSLIKMDCNKAEKFD
FVLQYLNKMAGNEYMGFSNATFQSEKETGDRNYAIGYYLKEKKCFPKGVDMMAALDL
YFQLCSVEVTCESGSVMAATLANGGICPITGESVLSAEAVRNTLSLMHSCGMYDFSGQF
AFHVGLPAKSAVSGAILLVVPNVMGMMCLSPPLDKLGNSHRGTSFCQKLVSLFNFHNY
DNLRHCARKLDPRREGAEIRNKTVVNLLFAAYSGDVSALRRFALSAMDMEQKDYDSRT
ALHVAAAEGHIEVVKFLIEACKVNPFAKDRWGNIPLDDAVQFNHLEVVKLLQDYQDSY
TLSETQAEAAAEALSKENLESMV
MetArgSerMetLysAlaLeuGlnLysAlaLeuSerArgAlaGlySerHisCysGlyArgGlyGlyTrp
GlyHisProSerArgSerProLeuLeuGlyGlyGlyValArgHisHisLeuSerGluAlaAlaAlaGlnGlyArgGlu
ThrProHisSerHisGlnProGlnHisGlnAspHisAspSerSerGluSerGlyMetLeuSerArgLeuGlyAspLeu
LeuPheTyrThrIleAlaGluGlyGlnGluArgIleProIleHisLysPheThrThrAlaLeuLysAlaThrGlyLeuGln
ThrSerAspProArgLeuArgAspCysMetSerGluMetHisArgValValGlnGluSerSerSerGlyGlyLeuLeu
AspArgAspLeuPheArgLysCysValSerSerAsnIleValLeuLeuThrGlnAlaPheArgLysLysPheValIlePro
AspPheGluGluPheThrGlyHisValAspArgIlePheGluAspValLysGluLeuThrGlyGlyLysValAlaAla
TyrIleProGlnLeuAlaLysSerAsnProAspLeuTrpGlyValSerLeuCysThrValAspGlyGlnArgHisSer
ValGlyHisThrLysIleProPheCysLeuGlnSerCysValLysProLeuThrTyrAlaIleSerIleSerThrLeuGlyThr
AspTyrValHisLysPheValGlyLysGluProSerGlyLeuArgTyrAsnLysLeuSerLeuAsnGluGluGlyIle
ProHisAsnProMetValAsnAlaGlyAlaIleValValSerSerLeuIleLysMetAspCysAsnLysAlaGluLysPhe
AspPheValLeuGlnTyrLeuAsnLysMetAlaGlyAsnGluTyrMetGlyPheSerAsnAlaThrPheGlnSer
GluLysGluThrGlyAspArgAsnTyrAlaIleGlyTyrTyrLeuLysGluLysLysCysPheProLysGlyValAsp
MetMetAlaAlaLeuAspLeuTyrPheGlnLeuCysSerValGluValThrCysGluSerGlySerValMetAlaAlaThr
LeuAlaAsnGlyGlyIleCysProIleThrGlyGluSerValLeuSerAlaGluAlaValArgAsnThrLeuSerLeu
MetHisSerCysGlyMetTyrAspPheSerGlyGlnPheAlaPheHisValGlyLeuProAlaLysSerAlaValSerGly
AlaIleLeuLeuValValProAsnValMetGlyMetMetCysLeuSerProProLeuAspLysLeuGlyAsnSerHis
ArgGlyThrSerPheCysGlnLysLeuValSerLeuPheAsnPheHisAsnTyrAspAsnLeuArgHisCysAlaArg
LysLeuAspProArgArgGluGlyAlaGluIleArgAsnLysThrValValAsnLeuLeuPheAlaAlaTyrSerGly
AspValSerAlaLeuArgArgPheAlaLeuSerAlaMetAspMetGluGlnLysAspTyrAspSerArgThrAlaLeu
HisValAlaAlaAlaGluGlyHisIleGluValValLysPheLeuIleGluAlaCysLysValAsnProPheAlaLys
AspArgTrpGlyAsnIleProLeuAspAspAlaValGlnPheAsnHisLeuGluValValLysLeuLeuGlnAspTyr
GlnAspSerTyrThrLeuSerGluThrGlnAlaGluAlaAlaAlaGluAlaLeuSerLysGluAsnLeuGluSerMetVal
Protein: Pik3c3

| | |
|---|---|
| <210> | 22 |
| <211> | 887 |
| <212> | PRT |
| <213> | Homo sapiens |
| <221> | WIPO Standard ST.26 |
| <222> | 1...88 |
| <400> | 22 |

MGEAEKFHYIYSCDLDINVQLKIGSLEGKREQKSYKAVLEDPMLKFSGLYQE
TCSDLYVTCQVFAEGKPLALPVRTSYKAFSTRWNWNEWLKLPVKYPDLPRNAQVALTI
WDVYGPGKAVPVGGTTVSLFGKYGMFRQGMHDLKVWPNVEADGSEPTKTPGRTSSTL
SEDQMSRLAKLTKAHRQGHMVKVDWLDRLTFREIEMINESEKRSSNFMYLMVEFRCV
KCDDKEYGIVYYEKDGDESSPILTSFELVKVPDPQMSMENLVESKHHKLARSLRSGPSD
HDLKPNAATRDQLNIIVSYPPTKQLTYEEQDLVWKFRYYLTNQEKALTKFLKCVNWDL
PQEAKQALELLGKWKPMDVEDSLELLSSHYTNPTVRRYAVARLRQADDEDLLMYLLQ
LVQALKYENFDDIKNGLEPTKKDSQSSVSENVSNSGINSAEIDSSQIITSPLPSVSSPPPASK
TKEVPDGENLEQDLCTFLISRACKNSTLANYLYWYVIVECEDQDTQQRDPKTHEMYLN
VMRRFSQALLKGDKSVRVMRSLLAAQQTFVDRLVHLMKAVQRESGNRKKKNERLQA
LLGDNEKMNLSDVELIPLPLEPQVKIRGIIPETATLFKSALMPAQLFFKTEDGGKYPVIFK
HGDDLRQDQLILQIISLMDKLLRKENLDLKLTPYKVLATSTKHGFMQFIQSVPVAEVLDT
EGSIQNFFRKYAPSENGPNGISAEVMDTYVKSCAGYCVITYILGVGDRHLDNLLLTKTG
KLFHIDFGYILGRDPKPLPPPMKLNKEMVEGMGGTQSEQYQEFRKQCYTAFLHLRRYSN
LILNLFSLMVDANIPDIALEPDKTVKKVQDKFRLDLSDEEAVHYMQSLIDESVHALFAAV
VEQIHKFAQYWRK
MetGlyGluAlaGluLysPheHisTyrIleTyrSerCysAspLeuAspIleAsnValGlnLeuLysIle
GlySerLeuGluGlyLysArgGluGlnLysSerTyrLysAlaValLeuGluAspProMetLeuLysPheSerGlyLeu
TyrGlnGluThrCysSerAspLeuTyrValThrCysGlnValPheAlaGluGlyLysProLeuAlaLeuProValArgThr
SerTyrLysAlaPheSerThrArgTrpAsnTrpAsnGluTrpLeuLysLeuProValLysTyrProAspLeuProArg
AsnAlaGlnValAlaLeuThrIleTrpAspValTyrGlyProGlyLysAlaValProValGlyGlyThrThrValSer
LeuPheGlyLysTyrGlyMetPheArgGlnGlyMetHisAspLeuLysValTrpProAsnValGluAlaAspGlySer
GluProThrLysThrProGlyArgThrSerSerThrLeuSerGluAspGlnMetSerArgLeuAlaLysLeuThrLysAla
HisArgGlnGlyHisMetValLysValAspTrpLeuAspArgLeuThrPheArgGluIleGluMetIleAsnGluSer
GluLysArgSerSerAsnPheMetTyrLeuMetValGluPheArgCysValLysCysAspAspLysGluTyrGlyIle
ValTyrTyrGluLysAspGlyAspGluSerSerProIleLeuThrSerPheGluLeuValLysValProAspProGln
MetSerMetGluAsnLeuValGluSerLysHisHisLysLeuAlaArgSerLeuArgSerGlyProSerAspHisAsp
LeuLysProAsnAlaAlaThrArgAspGlnLeuAsnIleIleValSerTyrProProThrLysGlnLeuThrTyrGluGlu
GlnAspLeuValTrpLysPheArgTyrTyrLeuThrAsnGlnGluLysAlaLeuThrLysPheLeuLysCysValAsn
TrpAspLeuProGlnGluAlaLysGlnAlaLeuGluLeuLeuGlyLysTrpLysProMetAspValGluAspSer
LeuGluLeuLeuSerSerHisTyrThrAsnProThrValArgArgTyrAlaValAlaArgLeuArgGlnAlaAspAsp
GluAspLeuLeuMetTyrLeuLeuGlnLeuValGlnAlaLeuLysTyrGluAsnPheAspAspIleLysAsnGlyLeu
GluProThrLysLysAspSerGlnSerSerValSerGluAsnValSerAsnSerGlyIleAsnSerAlaGluIleAspSer
SerGlnIleIleThrSerProLeuProSerValSerSerProProProAlaSerLysThrLysGluValProAspGlyGlu
AsnLeuGluGlnAspLeuCysThrPheLeuIleSerArgAlaCysLysAsnSerThrLeuAlaAsnTyrLeuTyrTrp
TyrValIleValGluCysGluAspGlnAspThrGlnGlnArgAspProLysThrHisGluMetTyrLeuAsnValMet
ArgArgPheSerGlnAlaLeuLeuLysGlyAspLysSerValArgValMetArgSerLeuLeuAlaAlaGlnGlnThr
PheValAspArgLeuValHisLeuMetLysAlaValGlnArgGluSerGlyAsnArgLysLysLysAsnGluArgLeu
GlnAlaLeuLeuGlyAspAsnGluLysMetAsnLeuSerAspValGluLeuIleProLeuProLeuGluProGlnValL
ysIleArgGlyIleIleProGluThrAlaThrLeuPheLysSerAlaLeuMetProAlaGlnLeuPhePheLysThrGlu
AspGlyGlyLysTyrProValIlePheLysHisGlyAspAspLeuArgGlnAspGlnLeuIleLeuGlnIleIleSerLeu
MetAspLysLeuLeuArgLysGluAsnLeuAspLeuLysLeuThrProTyrLysValLeuAlaThrSerThrLys
HisGlyPheMetGlnPheIleGlnSerValProValAlaGluValLeuAspThrGluGlySerIleGlnAsnPhePheArg
LysTyrAlaProSerGluAsnGlyProAsnGlyIleSerAlaGluValMetAspThrTyrValLysSerCysAlaGlyTyr
CysValIleThrTyrIleLeuGlyValGlyAspArgHisLeuAspAsnLeuLeuLeuThrLysThrGlyLysLeuPhe
HisIleAspPheGlyTyrIleLeuGlyArgAspProLysProLeuProProProMetLysLeuAsnLysGluMetVal
GluGlyMetGlyGlyThrGlnSerGluGlnTyrGlnGluPheArgLysGlnCysTyrThrAlaPheLeuHisLeuArg
ArgTyrSerAsnLeuIleLeuAsnLeuPheSerLeuMetValAspAlaAsnIleProAspIleAlaLeuGluProAspLys
ThrValLysLysValGlnAspLysPheArgLeuAspLeuSerAspGluGluAlaValHisTyrMetGlnSerLeuIle
AspGluSerValHisAlaLeuPheAlaAlaValValGluGlnIleHisLysPheAlaGlnTyrTrpArgLys
Protein: ATG5

| | |
|---|---|
| <210> | 23 |
| <211> | 275 |
| <212> | PRT |
| <213> | Homo sapiens |
| <221> | WIPO Standard ST.26 |
| <222> | 1...275 |
| <400> | 23 |

MTDDKDVLRDVWFGRIPTCFTLYQDEITEREAEPYYLLLPRVSYLTLVTDKV
KKHFQKVMRQEDISEIWFEYEGTPLKWHYPIGLLFDLLASSSALPWNITVHFKSFPEKDL
LHCPSKDAIEAHFMSCMKEADALKHKSQVINEMQKKDHKQLWMGLQNDRFDQFWAI
NRKLMEYPAEENGFRYIPFRIYQTTTERPFIQKLFRPVAADGQLHTLGDLLKEVCPSAIDP
EDGEKKNQVMIHGIEPMLETPLQWLSEHLSYPDNFLHISIIPQPTD
MetThrAspAspLysAspValLeuArgAspValTrpPheGlyArgIleProThrCysPheThrLeuTyr
GlnAspGluIleThrGluArgGluAlaGluProTyrTyrLeuLeuLeuProArgValSerTyrLeuThrLeuValThrA
spLysValLysLysHisPheGlnLysValMetArgGlnGluAspIleSerGluIleTrpPheGluTyrGluGlyThrPro
LeuLysTrpHisTyrProIleGlyLeuLeuPheAspLeuLeuAlaSerSerSerAlaLeuProTrpAsnIleThrValHis
PheLysSerPheProGluLysAspLeuLeuHisCysProSerLysAspAlaIleGluAlaHisPheMetSerCysMetL
ysGluAlaAspAlaLeuLysHisLysSerGlnValIleAsnGluMetGlnLysLysAspHisLysGlnLeuTrpMetGl
yLeuGlnAsnAspArgPheAspGlnPheTrpAlaIleAsnArgLysLeuMetGluTyrProAlaGluGluAsnGlyP
heArgTyrIleProPheArgIleTyrGlnThrThrThrGluArgProPheIleGlnLysLeuPheArgProValAlaAlaA
spGlyGlnLeuHisThrLeuGlyAspLeuLeuLysGluValCysProSerAlaIleAspProGluAspGlyGluLysLy
sAsnGlnValMetIleHisGlyIleGluProMetLeuGluThrProLeuGlnTrpLeuSerGluHisLeuSerTyrProA
spAsnPheLeuHisIleSerIleIleProGlnProThrAsp
Protein: Hamp

| | |
|---|---|
| <210> | 24 |
| <211> | 84 |
| <212> | PRT |
| <213> | Homo sapiens |
| <221> | WIPO Standard ST.26 |
| <222> | 1...84 |
| <400> | 24 |

MALSSQIWAACLLLLLLLASLTSGSVFPQQTGQLAELQPQDRAGARASWMP
MFQRRRRRDTHFPICIFCCGCCHRSKCGMCCKT
MetAlaLeuSerSerGlnIleTrpAlaAlaCysLeuLeuLeuLeuLeuLeuLeuAlaSerLeuThrSer
GlySerValPheProGlnGlnThrGlyGlnLeuAlaGluLeuGlnProGlnAspArgAlaGlyAlaArgAlaSerTrp
MetProMetPheGlnArgArgArgArgArgAspThrHisPheProIleCysIlePheCysCysGlyCysCysHisArgSer
LysCysGlyMetCysCysLysThr
Protein: SRB1

| | |
|---|---|
| <210> | 25 |
| <211> | 552 |
| <212> | PRT |
| <213> | Homo sapiens |
| <221> | WIPO Standard ST.26 |
| <222> | 1...552 |
| <400> | 25 |

MGCSAKARWAAGALGVAGLLCAVLGAVMIVMVPSLIKQQVLKNVRIDPSSL
SFNMWKEIPIPFYLSVYFFDVMNPSEILKGEKPQVRERGPYVYREFRHKSNITFNNNDTV
SFLEYRTFQFQPSKSHGSESDYIVMPNILVLGAAVMMENKPMTLKLIMTLAFTTLGERAF
MNRTVGEIMWGYKDPLVNLINKYFPGMFPFKDKFGLFAELNNSDSGLFTVFTGVQNISR
IHLVDKWNGLSKVDFWHSDQCNMINGTSGQMWPPFMTPESSLEFYSPEACRSMKLMY
KESGVFEGIPTYRFVAPKTLFANGSIYPPNEGFCPCLESGIQNVSTCRFSAPLFLSHPHFLN
ADPVLAEAVTGLHPNQEAHSLFLDIHPVTGIPMNCSVKLQLSLYMKSVAGIGQTGKIEPV
VLPLLWFAESGAMEGETLHTFYTQLVLMPKVMHYAQYVLLALGCVLLLVPVICQIRSQ
VGAGORAARADSHSLACWGKGASDRTLWPTAAWSPPPAAVLRLCRSGSGHCWGLRST
LASFACRVATTLPVLEGLGPSLGGGTGS
MetGlyCysSerAlaLysAlaArgTrpAlaAlaGlyAlaLeuGlyValAlaGlyLeuLeuCysAlaVal
LeuGlyAlaValMetIleValMetValProSerLeuIleLysGlnGlnValLeuLysAsnValArgIleAspProSerSer
LeuSerPheAsnMetTrpLysGluIleProIleProPheTyrLeuSerValTyrPhePheAspValMetAsnProSer
GluIleLeuLysGlyGluLysProGlnValArgGluArgGlyProTyrValTyrArgGluPheArgHisLysSerAsnIle
ThrPheAsnAsnAsnAspThrValSerPheLeuGluTyrArgThrPheGlnPheGlnProSerLysSerHisGlySer
GluSerAspTyrIleValMetProAsnIleLeuValLeuGlyAlaAlaValMetMetGluAsnLysProMetThrLeuLys
LeuIleMetThrLeuAlaPheThrThrLeuGlyGluArgAlaPheMetAsnArgThrValGlyGluIleMetTrpGly
TyrLysAspProLeuValAsnLeuIleAsnLysTyrPheProGlyMetPheProPheLysAspLysPheGlyLeuPhe
AlaGluLeuAsnAsnSerAspSerGlyLeuPheThrValPheThrGlyValGlnAsnIleSerArgIleHisLeuValAsp
LysTrpAsnGlyLeuSerLysValAspPheTrpHisSerAspGlnCysAsnMetIleAsnGlyThrSerGlyGlnMet
TrpProProPheMetThrProGluSerSerLeuGluPheTyrSerProGluAlaCysArgSerMetLysLeuMetTyr
LysGluSerGlyValPheGluGlyIleProThrTyrArgPheValAlaProLysThrLeuPheAlaAsnGlySerIleTyr
ProProAsnGluGlyPheCysProCysLeuGluSerGlyIleGlnAsnValSerThrCysArgPheSerAlaProLeuPhe
LeuSerHisProHisPheLeuAsnAlaAspProValLeuAlaGluAlaValThrGlyLeuHisProAsnGlnGluAla
HisSerLeuPheLeuAspIleHisProValThrGlyIleProMetAsnCysSerValLysLeuGlnLeuSerLeuTyr
MetLysSerValAlaGlyIleGlyGlnThrGlyLysIleGluProValValLeuProLeuLeuTrpPheAlaGluSerGly
AlaMetGluGlyGluThrLeuHisThrPheTyrThrGlnLeuValLeuMetProLysValMetHisTyrAlaGlnTyr
ValLeuLeuAlaLeuGlyCysValLeuLeuLeuValProValIleCysGlnIleArgSerGlnValGlyAlaGlyGlnArg
AlaAlaArgAlaAspSerHisSerLeuAlaCysTrpGlyLysGlyAlaSerAspArgThrLeuTrpProThrAlaAlaT
rpSerProProProAlaAlaValLeuArgLeuCysArgSerGlySerGlyHisCysTrpGlyLeuArgSerThrLeuAla
SerPheAlaCysArgValAlaThrThrLeuProValLeuGluGlyLeuGlyProSerLeuGlyGlyGlyThrGlySer
Protein: TMPRSS2

| | |
|---|---|
| <210> | 26 |
| <211> | 492 |
| <212> | PRT |
| <213> | Homo sapiens |
| <221> | WIPO Standard ST.26 |
| <222> | 1...492 |
| <400> | 26 |

MALNSGSPPAIGPYYENHGYQPENPYPAQPTVVPTVYEVHPAQYYPSPVPQY
APRVLTQASNPVVCTQPKSPSGTVCTSKTKKALCITLTLGTFLVGAALAAGLLWKFMGS
KCSNSGIECDSSGTCINPSNWCDGVSHCPGGEDENRCVRLYGPNFILQVYSSQRKSWHP
VCQDDWNENYGRAACRDMGYKNNFYSSQGIVDDSGSTSFMKLNTSAGNVDIYKKLYH
SDACSSKAVVSLRCIACGVNLNSSRQSRIVGGESALPGAWPWQVSLHVQNVHVCGGSII
TPEWIVTAAHCVEKPLNNPWHWTAFAGILRQSFMFYGAGYQVEKVISHPNYDSKTKNN
DIALMKLQKPLTFNDLVKPVCLPNPGMMLQPEQLCWISGWGATEEKGKTSEVLNAAKV
LLIETQRCNSRYVYDNLITPAMICAGFLQGNVDSCQGDSGGPLVTSKNNIWWLIGDTSW
GSGCAKAYRPGVYGNVMVFTDWIYRQMRADG
MetAlaLeuAsnSerGlySerProProAlaIleGlyProTyrTyrGluAsnHisGlyTyrGlnProGlu
AsnProTyrProAlaGlnProThrValValProThrValTyrGluValHisProAlaGlnTyrTyrProSerProValPro
GlnTyrAlaProArgValLeuThrGlnAlaSerAsnProValValCysThrGlnProLysSerProSerGlyThrValCys
ThrSerLysThrLysLysAlaLeuCysIleThrLeuThrLeuGlyThrPheLeuValGlyAlaAlaLeuAlaAlaGly
LeuLeuTrpLysPheMetGlySerLysCysSerAsnSerGlyIleGluCysAspSerSerGlyThrCysIleAsnProSer
AsnTrpCysAspGlyValSerHisCysProGlyGlyGluAspGluAsnArgCysValArgLeuTyrGlyProAsnPhe
IleLeuGlnValTyrSerSerGlnArgLysSerTrpHisProValCysGlnAspAspTrpAsnGluAsnTyrGlyArg
AlaAlaCysArgAspMetGlyTyrLysAsnAsnPheTyrSerSerGlnGlyIleValAspAspSerGlySerThrSerPhe
MetLysLeuAsnThrSerAlaGlyAsnValAspIleTyrLysLysLeuTyrHisSerAspAlaCysSerSerLysAla
ValValSerLeuArgCysIleAlaCysGlyValAsnLeuAsnSerSerArgGlnSerArgIleValGlyGlyGluSerAla
LeuProGlyAlaTrpProTrpGlnValSerLeuHisValGlnAsnValHisValCysGlyGlySerIleIleThrProGlu
TrpIleValThrAlaAlaHisCysValGluLysProLeuAsnAsnProTrpHisTrpThrAlaPheAlaGlyIleLeuArg
GlnSerPheMetPheTyrGlyAlaGlyTyrGlnValGluLysValIleSerHisProAsnTyrAspSerLysThrLysAsn
AsnAspIleAlaLeuMetLysLeuGlnLysProLeuThrPheAsnAspLeuValLysProValCysLeuProAsnPro
GlyMetMetLeuGlnProGluGlnLeuCysTrpIleSerGlyTrpGlyAlaThrGluGluLysGlyLysThrSerGlu
ValLeuAsnAlaAlaLysValLeuLeuIleGluThrGlnArgCysAsnSerArgTyrValTyrAspAsnLeuIleThrPro
AlaMetIleCysAlaGlyPheLeuGlnGlyAsnValAspSerCysGlnGlyAspSerGlyGlyProLeuValThrSer
LysAsnAsnIleTrpTrpLeuIleGlyAspThrSerTrpGlySerGlyCysAlaLysAlaTyrArgProGlyValTyrGly
AsnValMetValPheThrAspTrpIleTyrArgGlnMetArgAlaAspGly

What is claimed is:

1. A prophylactic method for preventing heart injuries comprising:
administering an effective dose of trigonelline to a subject to prevent injury caused by at least one virus;
wherein administration of trigonelline attenuates:
spike-protein exacerbated lipotoxicity and
mitochondrial dysfunction;
in at least one endothelial cell and at least one cardiomyocyte.

2. The method of claim 1, wherein the subject is obese.

3. The method of claim 1, wherein the at least one virus comprises severe acute respiratory syndrome coronavirus 2.

4. The method of claim 1, wherein the method attenuates mitochondrial dysfunction caused by low-density lipoprotein-cholesterol.

5. The method of claim 1, wherein the effective dose of trigonelline inhibits at least one spike protein.

6. The method of claim 5, wherein the at least one spike protein causes enhanced lipid deposition on at least one cell membrane.

7. The method of claim 5, wherein the at least one spike protein comprises SEQ ID NO: 1.

8. The method of claim 1, wherein ferroptiosis causes the spike-protein exacerbated lipotoxicity.

9. The method of claim 1, wherein Wortmannin is administered to inhibit protein-induced necrosis.

10. The method of claim 9, wherein the protein induced necrosis is caused by a protein comprising SEQ ID NO: 8.

11. A method for mitigating virus-induced cardiometabolic pathologies associated with obesity:
administering an effective dose of trigonelline to at least one host cell to prevent injury caused by at least one virus;
wherein administration of trigonelline attenuates:
spike-protein exacerbated lipotoxicity and
mitochondrial dysfunction;
in at least one endothelial cell and at least one cardiomyocyte.

12. The method of claim 11, wherein the host is obese.

13. The method of claim 11, wherein the at least one virus comprises severe acute respiratory syndrome coronavirus 2.

14. The method of claim 11, wherein the method attenuates mitochondrial dysfunction caused by low-density lipoprotein-cholesterol.

15. The method of claim 11, wherein the effective dose of trigonelline inhibits at least one spike protein.

16. The method of claim 15, wherein the at least one spike protein causes enhanced lipid deposition on at least one cell membrane.

17. The method of claim 15, wherein the at least one spike protein comprises SEQ ID NO: 1.

18. The method of claim 11, wherein ferroptiosis causes the spike-protein exacerbated lipotoxicity.

19. The method of claim 11, wherein Wortmannin is administered to inhibit protein-induced necrosis.

20. The method of claim 19, wherein the protein induced necrosis is caused by a protein comprising SEQ ID NO: 8.
